SEQUENCE LISTING (82 sequences)

SEQ ID NO: 1 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 2 - protein, 477 aa, Fusarium moniliforme
MASTSALPKQNPALRRTVTSTTVTDSESAAVSPSDSPRHSASSTSLSSMSEVDIAKPKSEYGVMLDTYGNQFEVPDFTIKDIYNAIPKHCFKRSALKGYGYILRDIVLLTTTFSIWYNFVTPEYIPSTPARAGLWAVYTVLQGLFGTGLWVIAHECGHGAFSDSRIINDITGWVLHSSLLVPYFSWQISHRKHHKATGNMERDMVFVPRTREQQATRLGKMTHELAHLTEETPAFTLLMLVLQQLVGWPNYLITNVTGHNYHERQREGRGKGKHNGLGGGVNHFDPRSPLYENSDAKLIVLSDIGIGLMATALYFLVQKFGFYNMAIWYFVPYLWVNHWLVAITFLQHTDPTLPHYTNDEWNFVRGAAATIDREMGFIGRHLLHGIIETHVLHHYVSSIPFYNADEATEAIKPIMGKHYRADVQDGPRGFIRAMYRSARMCQWVEPSAGAEGAGKGVLFFRNRNNVGTPPAVIKPVA

SEQ ID NO: 3 - DNA, 1449 bp, Yarrowia lipolytica
atggtcaaaaacgtagaccaagtagacttatcccaagtagacacaatcgcttcaggtagagatgtcaattacaaggtaaaatacaccagtggtgttaaaatgtctcaaggtgcatatgatgacaagggtagacatatttcagaacaaccttttacttgggccaattggcatcaacacatcaactggttgaacttcatattagttatcgctttgccattatcttcattcgctgcagccccttttgtatctttcaactggaaaacagctgcatttgccgttggttattacatgtgtaccggtttgggtattactgctggttatcatagaatgtgggctcacagagcatacaaagccgctttaccagtcagaattatattggccttattcggtggtggtgctgtagaaggttctattagatggtgggcttccagtcatagagttcatcacagatggactgattctaataaggatccttatgacgcaagaaagggtttttggttctcacactttggttggatgttgttagttccaaatcctaaaaacaagggtagaacagatatatcagacttgaataacgattgggttgtcagattgcaacataagtactacgtatacgttttggtctttatggctatcgtcttgccaaccttagtatgtggtttcggttggggtgactggaagggtggtttggtatatgctggtatcatgagatacacatttgttcaacaagtcaccttctgcgttaattctttagcacattggattggtgaacaaccatttgatgacagaagaacacctagagatcatgccttgactgctttagttacattcggtgaaggttatcacaattttcatcacgaattcccatccgattacagaaacgctttgatctggtaccaatacgaccctactaaatggttgatctggacattaaagcaagttggtttggcttgggatttgcaaacctttagtcaaaatgcaattgaacaaggtttggtccaacaaagacaaaagaaattggacaagtggagaaacaacttaaactggggtatcccaatagaacaattgcctgttatagaattcgaagaattccaagaacaagcaaagaccagagatttggttttaatttccggtatagtacatgacgttagtgcctttgtcgaacatcacccaggtggtaaagctttgattatgtccgcagttggtaaagatggtactgctgttttcaatggtggtgtctacagacattccaatgcaggtcacaacttgttagccaccatgagagtaagtgttattagaggtggtatggaagtcgaagtatggaagactgcacaaaacgaaaagaaagatcaaaacatcgtctctgacgaatcaggtaatagaattcatagagcaggtttacaagccacaagagtagaaaaccctggcatgtctggtatggcagcctaa

SEQ ID NO: 4 - DNA, 1434 bp, Fusarium moniliforme
atggcatccacatccgccttgccaaaacaaaatccagcattgagaagaaccgttacatccacaaccgttaccgacagtgaatccgccgcagtttctccatcagattcccctagacatagtgcatcttcaacatctttatccagtatgtcagaagtagatattgccaaaccaaagtctgaatatggtgttatgttggacacatacggtaaccaatttgaagtcccagatttcaccattaaagacatctataacgccatccctaagcattgtttcaagagatcagctttgaagggttacggttacatcttgagagatatcgtattgttgactacaaccttttccatctggtataatttcgttactcctgaatacattccatctacacctgctagagcaggtttatgggctgtatataccgttttgcaaggtttattcggtactggtttgtgggttattgcacatgaatgcggtcacggtgcctttagtgattctagaattataaacgacatcaccggttgggtcttacattcttcattgttagtaccatacttctcatggcaaatctcccacagaaaacatcacaaggccactggtaatatggaaagagatatggtttttgtccctagaactagagaacaacaagcaacaagattgggtaaaatgacccatgaattggctcacttaactgaagaaacaccagcattcacattgttgatgttggttttgcaacaattagtcggttggcctaattatttgattaccaacgttactggtcataattaccacgaaagacaaagagaaggtcgtggtaaaggtaaacataacggtttaggtggtggtgttaatcactttgatccaagatcccctttgtacgaaaacagtgatgctaagttgatagtcttgtctgacatcggtatcggtttaatggccactgctttgtactttttggtacaaaagttcggtttctacaacatggctatatggtatttcgtaccatacttgtgggttaatcattggttggtcgcaatcacatttttgcaacatacagatccaaccttacctcactacacaaatgacgaatggaactttgttagaggtgctgcagccaccattgatagagaaatgggtttcataggtagacatttgttacacggtatcattgaaactcatgtattgcatcactatgtttccagtattccattctacaacgctgacgaagcaacagaagccatcaaacctataatgggtaaacattacagagctgatgttcaagacggtccaagaggttttattagagctatgtacagatctgcaagaatgtgtcaatgggtcgaaccttcagcaggtgccgaaggtgctggtaaaggtgttttgtttttcagaaacagaaataacgtcggtactccacctgccgtcattaagccagtagcttaa

SEQ ID NO: 5 - protein, 390 aa, Lactobacillus plantarum
MLDKIIYKNLFSKAFDITIEVTYWDGQIERYGTGMPAVKVRLNKEIPIKLLTNQPTLVLGEAYMNGDIEVDGSIQELIASAYRQKDSFLTHNSFLKHLPKISHSEKSSTKDIQSHYDIGNDFYKLWLDDTMTYSCAYFEHDDDTLKQAQLNKVRHILNKLATQPGKRLLDVGSGWGTLLFMAADEFGLDATGITLSQEQYDYTQAQIKQRHLEEKVHVQLKDYREVTGQFDYVTSVGMFEHVGKENLGLYFNKIQAFLVPGGRALIHGITGQHEGAGVDPFINQYIFPGGYIPNVAENLKHIMAAKLQFSDIEPLRRHYQKTLEIWYHNYQQVEQQVVKNYGERFDRMWQLYLQACAAAFEAGNIDVIQYLLVKAPSGTGLPMTRHYIYD

SEQ ID NO: 6 - protein, 397 aa, Lactobacillus plantarum
MLEKTFYHTLLSHSFNMPVTVNYWDGSSETYGEGTPEVTVTFKEAIPMREITKNASIALGEAYMDGKIEIDGSIQKLIESAYESAESFFNNSKFKKFMPKQSHSEKKSQQDIQSHYDVGNDFYKMWLDPTMTYSCAYFKHDTDTLEEAQIHKVHHIIQKLNPQPGKTLLDIGCGWGTLMLTAAKEYGLKVVGVTLSQEQYNLVAQRIKDEGLSDVAEVRLQDYRELGDETFDYITSVGMFEHVGKDNLAMYFERVNHYLKADGVALLHGITRQQGGATNGWLDKYIFPGGYVPGMTENLQHIVDAGLQVADVETLRRHYQRTTEIWDKNFNAKRAAIEEKMGVRFTRMWDLYLQACAASFQSGNIDVMQYLVTKGASSRTLPMTRKYMYADNRINKA

SEQ ID NO: 7 - DNA, 1173 bp, Lactobacillus plantarum
atgttggataaaatcatctataaaaacttgttctctaaggcattcgatattaccattgaagtcacctactgggacggtcaaattgaaagatatggtactggtatgccagcagtaaaagttagattgaataaggaaataccaattaaattgttgacaaaccaacctaccttggtcttaggtgaagcttatatgaatggtgacatcgaagtagatggttccattcaagaattgatagcaagtgcctacagacaaaaggactcatttttgactcataactcattcttgaagcacttacctaagatttcccatagtgaaaaatcttcaacaaaggatatccaatctcattacgacatcggtaacgatttctacaagttgtggttggatgacactatgacatattcatgtgcatacttcgaacacgatgacgatactttgaagcaagcccaattgaataaggttagacatatcttgaacaagttagcaacacaaccaggtaaaagattgttagatgttggttccggttggggtaccttgttgtttatggctgcagacgaattcggtttggatgctaccggtataactttgagtcaagaacaatacgattacacacaagcacaaattaaacaaagacacttggaagaaaaggtccatgtacaattgaaggactacagagaagttaccggtcaatttgattacgttacttccgtcggcatgttcgaacacgtcggtaaagaaaatttgggtttgtacttcaacaaaattcaagccttcttggttccaggtggtagagctttaatccacggtattaccggtcaacatgaaggtgccggtgtagatccttttatcaaccaatacatattcccaggtggttacattcctaacgttgctgaaaacttgaagcatatcatggccgctaagttgcaattttctgatatcgaacctttgagaagacactaccaaaagacattagaaatctggtaccataactaccaacaagttgaacaacaagttgtcaaaaactatggtgaaagatttgacagaatgtggcaattgtacttacaagcttgcgcagccgctttcgaagctggtaacatcgatgtaatccaatacttgttagttaaggctccatcaggtactggtttacctatgacaagacattatatctatgattaa

SEQ ID NO: 8 - DNA, 1194 bp, Lactobacillus plantarum
atgttagaaaagacattttaccacaccttattatctcactccttcaacatgccagtcacagtcaactattgggacggttcctcagaaacttatggtgaaggtacaccagaagtaaccgttacttttaaggaagccatacctatgagagaaatcactaaaaatgcttccatagcattgggtgaagcatatatggatggtaaaatcgaaattgacggttccatacaaaaattaatcgaaagtgcctacgaatctgctgaatcatttttcaacaactctaagtttaaaaagtttatgccaaagcaatcccatagtgaaaagaaatctcaacaagatatccaatcacactacgatgttggtaacgacttctacaagatgtggttggaccctacaatgacctactcatgtgcatacttcaagcatgatactgacacattggaagaagcccaaatacacaaggttcatcacataatccaaaagttgaacccacaacctggtaaaaccttgttagatatcggttgcggttggggtaccttgatgttaactgctgcaaaagaatatggtttgaaggttgtcggtgtaacattgtctcaagaacaatacaacttggttgctcaaagaattaaagatgaaggtttgtcagacgtcgcagaagtaagattgcaagattacagagaattaggtgacgaaacctttgactacataacttctgtcggcatgttcgaacatgttggtaaagataatttggctatgtacttcgaaagagttaaccattacttaaaggcagacggtgtcgccttgttacacggtataacaagacaacaaggtggtgctaccaacggttggttggataagtacatcttcccaggtggttacgtccctggtatgactgaaaacttacaacatatagttgatgctggtttgcaagttgcagacgtcgaaacattgagaagacactaccaaagaactacagaaatctgggataagaacttcaacgctaagagagccgctattgaagaaaagatgggtgttagattcactagaatgtgggatttgtatttgcaagcttgtgcagcctcctttcaaagtggtaatattgacgtaatgcaatacttggttacaaaaggtgcatcttcaagaacattaccaatgaccagaaagtacatgtacgccgataacagaattaacaaagcttaa

SEQ ID NO: 9 - protein, 445 aa, Mortierella alpina
MATPLPPTFTVPASSTETRRDPLPHDVLPPLFNGEKVNILNIWKYLDWKHVIGLLVTPLVALYGMCTTELHTKTLVWSIVYYFATGLGITAGYHRLWAHRAYNAGPAMSFALALFGAGAVEGSIKWWSRGHRAHHRWTDTEKDPYSAHRGVFYSHLGWLLIKRPGWKIGHADVDDLNKNPLVQWQHKHYLILVILMGLVFPTAVAGLGWGDWRGGYFYAAILRLIFVHHATFCVNSLAHWLGDGPFDDRHTPRDHFITAFLTLGEGYHNFHHQFPQDYRSAIRFYQYDPTKWLIATCAFFGFASHLKTFPENEIKKGKLQMIEKEVLEKKTKLQWGTPIADLPILSFEDFQHACKNDRKQWILLEGVVYDVADFMTEHPGGEKYIKMGVGKDMTSAFNGGMYDHSNAARNLLSLMRVAVVEFGGEVEAQKSRPSVTVYGDHSKEE

SEQ ID NO: 10 - DNA, 1338 bp, Mortierella alpina
atggcaacacctttacctccaacattcactgtcccagcctcctccaccgaaaccagaagagaccctttacctcacgacgtattacctccattgtttaatggtgaaaaggttaacatattgaacatatggaaatatttggattggaagcatgtcattggtttgttagttactcctttggtcgctttatacggcatgtgtactacagaattgcacaccaagactttagtatggtccatagtttactacttcgcaaccggtttgggtataactgccggttatcatagattatgggcacacagagcctacaacgctggtccagcaatgagttttgcattggccttattcggtgctggtgcagttgaaggttccattaaatggtggagtagaggtcatagagcacatcacagatggacagataccgaaaaggacccttattctgcacatagaggtgttttctattcacacttaggttggttgttaatcaaaagaccaggttggaagattggtcatgctgatgtagatgacttgaataagaaccctttagttcaatggcaacataagcactatttgatcttagttattttgatgggtttagtcttcccaactgccgtagctggtttgggttggggtgactggagaggtggttacttctacgctgcaatcttgagattgatcttcgttcatcacgctacattctgcgtcaattccttggcacactggttaggtgacggtccatttgatgacagacatacccctagagatcactttattactgccttcttgacattaggtgaaggttatcataactttcatcaccaattcccacaagactacagatctgcaatcagattctatcaatacgatcctacaaaatggttgattgccacctgtgctttctttggttttgcttcacatttgaagacattcccagaaaacgaaattaagaaaggtaaattgcaaatgatcgaaaaggaagttttggaaaagaaaactaagttgcaatggggtacaccaatagcagatttgcctatcttgtctttcgaagacttccaacatgcctgcaagaacgatagaaagcaatggatcttgttagaaggtgttgtctatgatgttgcagactttatgaccgaacacccaggtggtgaaaaatacattaagatgggtgttggtaaagacatgacttctgctttcaacggtggcatgtatgatcattccaatgccgctagaaacttgttaagtttgatgagagtcgccgtagttgaatttggtggtgaagtagaagctcaaaaatctagaccttcagtcacagtatacggtgaccattcaaaggaagaataa

SEQ ID NO: 11 - protein, 258 aa, Euglena gracilis
MEVVNEIVSIGQEVLPKVDYAQLWSDASHCEVLYLSIAFVILKFTLGPLGPKGQSRMKFVFTNYNLLMSIYSLGSFLSMAYAMYTIGVMSDNCEKAFDNNVFRITTQLFYLSKFLEYIDSFYLPLMGKPLTWLQFFHHLGAPMDMWLFYNYRNEAVWIFVLLNGFIHWIMYGYYWTRLIKLKFPMPKSLITSMQIIQFNVGFYIVWKYRNIPCYRQDGMRMFGWFFNYFYVGTVLCLFLNFYVQTYIVRKHKGAKKIQ

SEQ ID NO: 12 - DNA, 777 bp, Euglena gracilis
atggaagtagtcaacgaaatagttagtatcggtcaagaagttttgcctaaagtagattacgcccaattatggtcagatgcctctcactgcgaagtattgtatttgtctatcgcattcgttatcttgaaattcactttaggtccattgggtcctaagggtcaatcaagaatgaagttcgttttcacaaactacaacttgttgatgtctatctattcattgggttcctttttgagtatggcttatgcaatgtacaccattggtgtcatgtctgataactgtgaaaaggccttcgacaacaacgttttcagaatcactacacaattattttatttgtccaaattcttggaatacatcgatagtttctacttgccattgatgggtaaacctttaacatggttgcaatttttccatcacttaggtgccccaatggacatgtggttgttttataactacagaaacgaagctgtatggatcttcgttttgttgaacggtttcatccattggatcatgtacggttactactggacaagattgattaaattgaagttcccaatgcctaagtctttgatcacctcaatgcaaatcatccaattcaatgtcggtttctacatagtatggaagtacagaaacataccttgttacagacaagatggtatgagaatgttcggttggtttttcaactacttctacgttggtaccgtcttgtgcttatttttgaacttctacgttcaaacttacatcgtcagaaaacacaagggtgctaaaaagattcaataa

SEQ ID NO: 13 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 14 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 15 - protein, 304 aa, Yarrowia lipolytica
MLSSISPDLYSSFSFKNSLAEAMPSVPHELINSKTLSWMYNASLDIRVPLTIGTIYAVSVHLTNSSERIKKRQPIAFAKTALFKWLCVLHNAGLCLYSAWTFVGILNAVKHAYQITGDSSAPFSFNTLWGSFCSRDSLWVTGLNYYGYWFYLSKFYEVVDTMIILAKGKPSSMLQTYHHTGAMFSMWAGIRFASPPIWIFVVFNSLIHTIMYFYYTLTTLKIKVPKILKASLTTAQITQIVGGGILAASHAFIYYKDHQTETVCSCLTTQGQFFALAVNVIYLSPLAYLFIAFWIRSYLKAKSN

SEQ ID NO: 16 - protein, 275 aa, Mortierella alpina
MESGPMPAGIPFPEYYDFFMDWKTPLAIAATYTAAVGLFNPKVGKVSRVVAKSANAKPAERTQSGAAMTAFVFVHNLILCVYSGITFYYMFPAMVKNFRTHTLHEAYCDTDQSLWNNALGYWGYLFYLSKFYEVIDTIIIILKGRRSSLLQTYHHAGAMITMWSGINYQATPIWIFVVFNSFIHTIMYCYYAFTSIGFHPPGKKYLTSMQITQFLVGITIAVSYLFVPGCIRTPGAQMAVWINVGYLFPLTYLFVDFAKRTYSKRSAIAAQKKAQ

SEQ ID NO: 17 - DNA, 915 bp, Yarrowia lipolytica
atgttgtcctcaatctctcctgacttgtattcatcattctctttcaaaaactcattagccgaagccatgccttccgttccacacgaattgattaattcaaagactttgtcctggatgtacaacgcaagtttggatataagagttccattgaccataggtactatctacgccgtctctgtacatttgacaaattcttcagaaagaatcaaaaagagacaacctattgcctttgctaaaaccgccttgttcaagtggttgtgtgtcttacataatgccggtttgtgcttatatagtgcttggacattcgtaggtatcttgaacgctgttaagcacgcataccaaataaccggtgactcttctgcaccattttctttcaatactttgtggggttccttctgtagtagagactctttatgggttactggtttgaactactacggttactggttctacttatctaagttctacgaagttgtcgatacaatgatcatcttggctaagggtaaaccttcttcaatgttgcaaacataccatcacaccggtgcaatgttttcaatgtgggccggtattagattcgcttccccacctatctggatttttgtagttttcaactcattgatacatactatcatgtacttctactacacattgactacattgaaaattaaggtcccaaagatcttgaaggcatccttgaccactgcccaaatcactcaaattgtaggtggtggtatattggctgcatcacatgcttttatctattacaaagaccaccaaactgaaacagtttgttcctgcttaacaacccaaggtcaatttttcgcattggccgttaacgtcatatatttgtctcctttggcttacttgtttatagcattctggatcagaagttatttgaaggctaagtctaattaa

SEQ ID NO: 18 - DNA, 828 bp, Mortierella alpina
atggaatctggtcctatgcctgctggtatcccttttcctgaatactacgacttctttatggattggaaaacacctttggctatcgctgccacttatacagctgcagttggtttattcaatccaaaggttggtaaagtttctagagttgtcgccaaatcagctaacgcaaagcctgctgaaagaactcaatctggtgccgctatgacagccttcgtcttcgtacataatttgatattgtgtgtttactcaggtatcacattctactacatgttcccagcaatggtcaaaaactttagaacccatactttacacgaagcatattgcgataccgaccaatctttatggaataacgccttgggttattggggttatttgttttatttgtcaaagttctacgaagttattgatactattataatcattttgaagggtagaagatcttcattgttacaaacctaccatcacgccggtgctatgataactatgtggtccggtatcaattatcaagctacaccaatctggatcttcgtagttttcaacagttttatccatacaatcatgtactgttactacgcattcacctccataggttttcacccacctggtaaaaagtatttgacaagtatgcaaataacccaattcttggttggtattaccatagctgtctcctatttgtttgtaccaggttgcatcagaactcctggtgcacaaatggccgtatggataaacgttggttacttgttccctttgacttatttgttcgttgacttcgctaaaagaacatactccaagagaagtgctattgcagcccaaaagaaagcacaataa

SEQ ID NO: 19 - protein, 554 aa, Lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 20 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaa

SEQ ID NO: 21 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 22 - DNA, 1665 bp, Lactococcus lactis
atgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattga

SEQ ID NO: 23 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 24 - protein, 330 aa, Methanococcus maripaludis
MKVFYDSDFKLDALKEKTIAVIGYGSQGRAQSLNMKDSGLNVVVGLRKNGASWNNAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILREIQDGRFTKEFLLEKQVSYAHLKSMRRLEGDLQIEEVGAKLRKMCGLEKEE

SEQ ID NO: 25 - protein, 342 aa, Bacillus subtilis
MVKVYYNGDIKENVLAGKTVAVIGYGSQGHAHALNLKESGVDVIVGVRQGKSFTQAQEDGHKVFSVKEAAAQAEIIMVLLPDEQQQKVYEAEIKDELTAGKSLVFAHGFNVHFHQIVPPADVDVFLVAPKGPGHLVRRTYEQGAGVPALFAIYQDVTGEARDKALAYAKGIGGARAGVLETTFKEETETDLFGEQAVLCGGLSALVKAGFETLTEAGYQPELAYFECLHELKLIVDLMYEEGLAGMRYSISDTAQWGDFVSGPRVVDAKVKESMKEVLKDIQNGTFAKEWIVENQVNRPRFNAINASENEHQIEVVGRKLREMMPFVKQGKKKEAVVSVAQN

SEQ ID NO: 26 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 27 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 28 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatggaacaacgctaaagcagacggtcacaatgtaatgaccattgaagaagctgctgaaaaagcggacatcatccacatcttaatacctgatgaattacaggcagaagtttatgaaagccagataaaaccatacctaaaagaaggaaaaacactaagcttttcacatggttttaacatccactatggattcattgttccaccaaaaggagttaacgtggttttagttgctccaaaatcacctggaaaaatggttagaagaacatacgaagaaggtttcggtgttccaggtttaatctgtattgaaattgatgcaacaaacaacgcatttgatattgtttcagcaatggcaaaaggaatcggtttatcaagagctggagttatccagacaactttcaaagaagaaacagaaactgaccttttcggtgaacaagctgttttatgcggtggagttaccgaattaatcaaggcaggatttgaaacactcgttgaagcaggatacgcaccagaaatggcatactttgaaacctgccacgaattgaaattaatcgttgacttaatctaccaaaaaggattcaaaaacatgtggaacgatgtaagtaacactgcagaatacggcggacttacaagaagaagcagaatcgttacagctgattcaaaagctgcaatgaaagaaatcttaagagaaatccaagatggaagattcacaaaagaattccttctcgaaaaacaggtaagctatgctcatttaaaatcaatgagaagactcgaaggagacttacaaatcgaagaagtcggcgcaaaattaagaaaaatgtgcggtcttgaaaaagaagaataa

SEQ ID NO: 29 - DNA, 1476 bp, Bacillus subtilis; identical to SEQ ID NO: 26

SEQ ID NO: 30 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 31 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 32 - protein, 550 aa, Methanococcus maripaludis
MISDNVKKGVIRTPNRALLKACGYTDEDMEKPFIGIVNSFTEVVPGHIHLRTLSEAAKHGVYANGGTPFEFNTIGICDGIAMGHEGMKYSLPSREIIADAVESMARAHGFDGLVLIPTCDKIVPGMIMGALRLNIPFIVVTGGPMLPGEFQGKKYELISLFEGVGEYQVGKITEEELKCIEDCACSGAGSCAGLYTANSMACLTEALGLSLPMCATTHAVDAQKVRLAKKSGSKIVDMVKEDLKPTDILTKEAFENAILVDLALGGSTNTTLHIPAIANEIENKFITLDDFDRLSDEVPHIASIKPGGEHYMIDLHNAGGIPAVLNVLKEKIRDTKTVDGRSILEIAESVKYINYDVIRKVEAPVHETAGLRVLKGNLAPNGCVVKIGAVHPKMYKHDGPAKVYNSEDEAISAILGGKIVEGDVIVIRYEGPSGGPGMREMLSPTSAICGMGLDDSVALITDGRFSGGSRGPCIGHVSPEAAAGGVIAAIENGDIIKIDMIEKEINVDLDESVIKERLSKLGEFEPKIKKGYLSRYSKLVSSADEGAVLK

SEQ ID NO: 33 - protein, 558 aa, Bacillus subtilis
MAELRSNMITQGIDRAPHRSLLRAAGVKEEDFGKPFIAVCNSYIDIVPGHVHLQEFGKIVKEAIREAGGVPFEFNTIGVDDGIAMGHIGMRYSLPSREIIADSVETVVSAHWFDGMVCIPNCDKITPGMLMAAMRINIPTIFVSGGPMAAGRTSYGRKISLSSVFEGVGAYQAGKINENELQELEQFGCPTCGSCSGMFTANSMNCLSEALGLALPGNGTILATSPERKEFVRKSAAQLMETIRKDIKPRDIVTVKAIDNAFALDMALGGSTNTVLHTLALANEAGVEYSLERINEVAERVPHLAKLAPASDVFIEDLHEAGGVSAALNELSKKEGALHLDALTVTGKTLGETIAGHEVKDYDVIHPLDQPFTEKGGLAVLFGNLAPDGAIIKTGGVQNGITRHEGPAVVFDSQDEALDGIINRKVKEGDVVIIRYEGPKGGPGMPEMLAPTSQIVGMGLGPKVALITDGRFSGASRGLSIGHVSPEAAEGGPLAFVENGDHIIVDIEKRILDVQVPEEEWEKRKANWKGFEPKVKTGYLARYSKLVTSANTGGIMKI

SEQ ID NO: 34 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 35 - DNA, 1758 bp, Saccharomyces cerevisiae
atgggcttgttaacgaaagttgctacatctagacaattctctacaacgagatgcgttgcaaagaagctcaacaagtactcgtatatcatcactgaacctaagggccaaggtgcgtcccaggccatgctttatgccaccggtttcaagaaggaagatttcaagaagcctcaagtcggggttggttcctgttggtggtccggtaacccatgtaacatgcatctattggacttgaataacagatgttctcaatccattgaaaaagcgggtttgaaagctatgcagttcaacaccatcggtgtttcagacggtatctctatgggtactaaaggtatgagatactcgttacaaagtagagaaatcattgcagactcctttgaaaccatcatgatggcacaacactacgatgctaacatcgccatcccatcatgtgacaaaaacatgcccggtgtcatgatggccatgggtagacataacagaccttccatcatggtatatggtggtactatcttgcccggtcatccaacatgtggttcttcgaagatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttga

SEQ ID NO: 36 - DNA, 1653 bp, Methanococcus maripaludis
atgataagtgataacgtcaaaaagggagttataagaactccaaaccgagctcttttaaaggcttgcggatatacagacgaagacatggaaaaaccatttattggaattgtaaacagctttacagaagttgttcccggccacattcacttaagaacattatcagaagcggctaaacatggtgtttatgcaaacggtggaacaccatttgaatttaataccattggaatttgcgacggtattgcaatgggccacgaaggtatgaaatactctttaccttcaagagaaattattgcagacgctgttgaatcaatggcaagagcacatggatttgatggtcttgttttaattcctacgtgtgataaaatcgttcctggaatgataatgggtgctttaagactaaacattccatttattgtagttactggaggaccaatgcttcccggagaattccaaggtaaaaaatacgaacttatcagcctttttgaaggtgtcggagaataccaagttggaaaaattactgaagaagagttaaagtgcattgaagactgtgcatgttcaggtgctggaagttgtgcagggctttacactgcaaacagtatggcctgccttacagaagctttgggactctctcttccaatgtgtgcaacaacgcatgcagttgatgcccaaaaagttaggcttgctaaaaaaagtggctcaaaaattgttgatatggtaaaagaagacctaaaaccaacagacatattaacaaaagaagcttttgaaaatgctattttagttgaccttgcacttggtggatcaacaaacacaacattacacattcctgcaattgcaaatgaaattgaaaataaattcataactctcgatgactttgacaggttaagcgatgaagttccacacattgcatcaatcaaaccaggtggagaacactacatgattgatttacacaatgctggaggtattcctgcggtattgaacgttttaaaagaaaaaattagagatacaaaaacagttgatggaagaagcattttggaaatcgcagaatctgttaaatacataaattacgacgttataagaaaagtggaagctccggttcacgaaactgctggtttaagggttttaaagggaaatcttgctccaaacggttgcgttgtaaaaatcggtgcagtacatccgaaaatgtacaaacacgatggacctgcaaaagtttacaattccgaagatgaagcaatttctgcgatacttggcggaaaaattgtagaaggggacgttatagtaatcagatacgaaggaccatcaggaggccctggaatgagagaaatgctctccccaacttcagcaatctgtggaatgggtcttgatgacagcgttgcattgattactgatggaagattcagtggtggaagtaggggcccatgtatcggacacgtttctccagaagctgcagctggcggagtaattgctgcaattgaaaacggggatatcatcaaaatcgacatgattgaaaaagaaataaatgttgatttagatgaatcagtcattaaagaaagactctcaaaactgggagaatttgagcctaaaatcaaaaaaggctatttatcaagatactcaaaacttgtctcatctgctgacgaaggggcagttttaaaataa

SEQ ID NO: 37 - DNA, 1677 bp, Bacillus subtilis
atggcagaattacgcagtaatatgatcacacaaggaatcgatagagctccgcaccgcagtttgcttcgtgcagcaggggtaaaagaagaggatttcggcaagccgtttattgcggtgtgtaattcatacattgatatcgttcccggtcatgttcacttgcaggagtttgggaaaatcgtaaaagaagcaatcagagaagcagggggcgttccgtttgaatttaataccattggggtagatgatggcatcgcaatggggcatatcggtatgagatattcgctgccaagccgtgaaattatcgcagactctgtggaaacggttgtatccgcacactggtttgacggaatggtctgtattccgaactgcgacaaaatcacaccgggaatgcttatggcggcaatgcgcatcaacattccgacgatttttgtcagcggcggaccgatggcggcaggaagaacaagttacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcaacatctccggaacgcaaagagtttgtgagaaaatcggctgcgcaattaatggaaacgattcgcaaagatatcaaaccgcgtgatattgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggctaagctggcgcctgcatcggatgtgtttattgaagatcttcacgaagcgggcggcgtttcagcggctctgaatgagctttcgaagaaagaaggagcgcttcatttagatgcgctgactgttacaggaaaaactcttggagaaaccattgccggacatgaagtaaaggattatgacgtcattcacccgctggatcaaccattcactgaaaagggaggccttgctgttttattcggtaatctagctccggacggcgctatcattaaaacaggcggcgtacagaatgggattacaagacacgaagggccggctgtcgtattcgattctcaggacgaggcgcttgacggcattatcaaccgaaaagtaaaagaaggcgacgttgtcatcatcagatacgaagggccaaaaggcggacctggcatgccggaaatgctggcgccaacatcccaaatcgttggaatgggactcgggccaaaagtggcattgattacggacggacgtttttccggagcctcccgtggcctctcaatcggccacgtatcacctgaggccgctgagggcgggccgcttgcctttgttgaaaacggagaccatattatcgttgatattgaaaaacgcatcttggatgtacaagtgccagaagaagagtgggaaaaacgaaaagcgaactggaaaggttttgaaccgaaagtgaaaaccggctacctggcacgttattctaaacttgtgacaagtgccaacaccggcggtattatgaaaatctag

SEQ ID NO: 38 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 39 - protein, 330 aa, Methanococcus maripaludis; identical to SEQ ID NO: 24

SEQ ID NO: 40 - DNA, 1662 bp, Lactococcus lactis
tctagacatatgtatactgtgggggattacctgctggatcgcctgcacgaactggggattgaagaaattttcggtgtgccaggcgattataacctgcagttcctggaccagattatctcgcacaaagatatgaagtgggtcggtaacgccaacgaactgaacgcgagctatatggcagatggttatgcccgtaccaaaaaagctgctgcgtttctgacgacctttggcgttggcgaactgagcgccgtcaacggactggcaggaagctacgccgagaacctgccagttgtcgaaattgttgggtcgcctacttctaaggttcagaatgaaggcaaatttgtgcaccatactctggctgatggggattttaaacattttatgaaaatgcatgaaccggttactgcggcccgcacgctgctgacagcagagaatgctacggttgagatcgaccgcgtcctgtctgcgctgctgaaagagcgcaagccggtatatatcaatctgcctgtcgatgttgccgcagcgaaagccgaaaagccgtcgctgccactgaaaaaagaaaacagcacctccaatacatcggaccaggaaattctgaataaaatccaggaatcactgaagaatgcgaagaaaccgatcgtcatcaccggacatgagatcatctcttttggcctggaaaaaacggtcacgcagttcatttctaagaccaaactgcctatcaccaccctgaacttcggcaaatctagcgtcgatgaagcgctgccgagttttctgggtatctataatggtaccctgtccgaaccgaacctgaaagaattcgtcgaaagcgcggactttatcctgatgctgggcgtgaaactgacggatagctccacaggcgcatttacccaccatctgaacgagaataaaatgatttccctgaatatcgacgaaggcaaaatctttaacgagcgcatccagaacttcgattttgaatctctgattagttcgctgctggatctgtccgaaattgagtataaaggtaaatatattgataaaaaacaggaggattttgtgccgtctaatgcgctgctgagtcaggatcgtctgtggcaagccgtagaaaacctgacacagtctaatgaaacgattgttgcggaacagggaacttcatttttcggcgcctcatccatttttctgaaatccaaaagccatttcattggccaaccgctgtgggggagtattggttatacctttccggcggcgctgggttcacagattgcagataaggaatcacgccatctgctgtttattggtgacggcagcctgcagctgactgtccaggaactggggctggcgatccgtgaaaaaatcaatccgatttgctttatcatcaataacgacggctacaccgtcgaacgcgaaattcatggaccgaatcaaagttacaatgacatcccgatgtggaactatagcaaactgccggaatcctttggcgcgacagaggatcgcgtggtgagtaaaattgtgcgtacggaaaacgaatttgtgtcggttatgaaagaagcgcaggctgacccgaatcgcatgtattggattgaactgatcctggcaaaagaaggcgcaccgaaagttctgaaaaagatggggaaactgtttgcggagcaaaataaaagctaaggatcc

SEQ ID NO: 41 - DNA, 1647 bp, Lactococcus lactis
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 42 - DNA, 1644 bp, Lactococcus lactis
atgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatag

SEQ ID NO: 43 - protein, 382 aa, Escherichia coli
MSSSCIEEVSVPDDNWYRIANELLSRAGIAINGSAPADIRVKNPDFFKRVLQEGSLGLGESYMDGWWECDRLDMFFSKVLRAGLENQLPHHFKDTLRIAGARLFNLQSKKRAWIVGKEHYDLGNDLFSRMLDPFMQYSCAYWKDADNLESAQQAKLKMICEKLQLKPGMRVLDIGCGWGGLAHYMASNYDVSVVGVTISAEQQKMAQERCEGLDVTILLQDYRDLNDQFDRIVSVGMFEHVGPKNYDTYFAVVDRNLKPEGIFLLHTIGSKKTDLNVDPWINKYIFPNGCLPSVRQIAQSSEPHFVMEDWHNFGADYDTTLMAWYERFLAAWPEIADNYSERFKRMFTYYLNACAGAFRARDIQLWQVVFSRGVENGLRVAR

SEQ ID NO: 44 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 45 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 46 - protein, 533 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVI

SEQ ID NO: 47 - DNA, 1692 bp, Saccharomyces cerivisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 48 - DNA, 1692 bp, Saccharomyces cerivisiae
atgtctgaaataaccttaggtaaatatttatttgaaagattgagccaagtcaactgtaacaccgtcttcggtttgccaggtgactttaacttgtctcttttggataagctttatgaagtcaaaggtatgagatgggctggtaacgctaacgaattgaacgctgcctatgctgctgatggttacgctcgtatcaagggtatgtcctgtattattaccaccttcggtgttggtgaattgtctgctttgaatggtattgccggttcttacgctgaacatgtcggtgttttgcacgttgttggtgttccatccatctcttctcaagctaagcaattgttgttgcatcataccttgggtaacggtgacttcactgttttccacagaatgtctgccaacatttctgaaaccactgccatgatcactgatattgctaacgctccagctgaaattgacagatgtatcagaaccacctacactacccaaagaccagtctacttgggtttgccagctaacttggttgacttgaacgtcccagccaagttattggaaactccaattgacttgtctttgaagccaaacgacgctgaagctgaagctgaagttgttagaactgttgttgaattgatcaaggatgctaagaacccagttatcttggctgatgcttgtgcttctagacatgatgtcaaggctgaaactaagaagttgatggacttgactcaattcccagtttacgtcaccccaatgggtaagggtgctattgacgaacaacacccaagatacggtggtgtttacgttggtaccttgtctagaccagaagttaagaaggctgtagaatctgctgatttgatattgtctatcggtgctttgttgtctgatttcaataccggttctttctcttactcctacaagaccaaaaatatcgttgaattccactctgaccacatcaagatcagaaacgccaccttcccaggtgttcaaatgaaatttgccttgcaaaaattgttggatgctattccagaagtcgtcaaggactacaaacctgttgctgtcccagctagagttccaattaccaagtctactccagctaacactccaatgaagcaagaatggatgtggaaccatttgggtaacttcttgagagaaggtgatattgttattgctgaaaccggtacttccgccttcggtattaaccaaactactttcccaacagatgtatacgctatcgtccaagtcttgtggggttccattggtttcacagtcggcgctctattgggtgctactatggccgctgaagaacttgatccaaagaagagagttattttattcattggtgacggttctctacaattgactgttcaagaaatctctaccatgattagatggggtttgaagccatacatttttgtcttgaataacaacggttacaccattgaaaaattgattcacggtcctcatgccgaatataatgaaattcaaggttgggaccacttggccttattgccaacttttggtgctagaaactacgaaacccacagagttgctaccactggtgaatgggaaaagttgactcaagacaaggacttccaagacaactctaagattagaatgattgaagttatgttgccagtctttgatgctccacaaaacttggttaaacaagctcaattgactgccgctactaacgctaaacaataa

SEQ ID NO: 49 - DNA, 1692 bp, Saccharomyces cerivisiae
atgtctgaaattactcttggaaaatacttatttgaaagattgaagcaagttaatgttaacaccatttttgggctaccaggcgacttcaacttgtccctattggacaagatttacgaggtagatggattgagatgggctggtaatgcaaatgagctgaacgccgcctatgccgccgatggttacgcacgcatcaagggtttatctgtgctggtaactacttttggcgtaggtgaattatccgccttgaatggtattgcaggatcgtatgcagaacacgtcggtgtactgcatgttgttggtgtcccctctatctccgctcaggctaagcaattgttgttgcatcataccttgggtaacggtgattttaccgtttttcacagaatgtccgccaatatctcagaaactacatcaatgattacagacattgctacagccccttcagaaatcgataggttgatcaggacaacatttataacacaaaggcctagctacttggggttgccagcgaatttggtagatctaaaggttcctggttctcttttggaaaaaccgattgatctatcattaaaacctaacgatcccgaagctgaaaaggaagttattgataccgtactagaattgatccagaattcgaaaaaccctgttatactatcggatgcctgtgcttctaggcacaacgttaaaaaagaaacccagaagttaattgatttgacgcaattcccagcttttgtgacacctctaggtaaagggtcaatagatgaacagcatcccagatatggcggtgtttatgtgggaacgctgtccaaacaagacgtgaaacaggccgttgagtcggctgatttgatcctttcggtcggtgctttgctctctgattttaacacaggttcgttttcctactcctacaagactaaaaatgtagtggagtttcattccgattacgtaaaggtgaagaacgctacgttcctcggtgtacaaatgaaatttgcactacaaaacttactgaaggttattcccgatgttgttaagggctacaagagcgttcccgtaccaaccaaaactcccgcaaacaaaggtgtacctgctagcacgcccttgaaacaagagtggttgtggaacgaattgtccaaattcttgcaagaaggtgatgttatcatttccgagaccggcacgtctgccttcggtatcaatcaaactatctttcctaaggacgcctacggtatctcgcaggtgttgtgggggtccatcggttttacaacaggagcaactttaggtgctgcctttgccgctgaggagattgaccccaacaagagagtcatcttattcataggtgacgggtctttgcagttaaccgtccaagaaatctccaccatgatcagatgggggttaaagccgtatctttttgtccttaacaacgacggctacactatcgaaaagctgattcatgggcctcacgcagagtacaacgaaatccagacctgggatcacctcgccctgttgcccgcatttggtgcgaaaaagtacgaaaatcacaagatcgccactacgggtgagtgggatgccttaaccactgattcagagttccagaaaaactcggtgatcagactaattgaactgaaactgcccgtctttgatgctccggaaagtttgatcaaacaagcgcaattgactgccgctacaaatgccaaacaataa

SEQ ID NO: 50 - DNA, 1692 bp, Candida glabrata
atgtctgagattactttgggtagatacttgttcgagagattgaaccaagtcgacgttaagaccatcttcggtttgccaggtgacttcaacttgtccctattggacaagatctacgaagttgaaggtatgagatgggctggtaacgctaacgaattgaacgctgcttacgctgctgacggttacgctagaatcaagggtatgtcctgtatcatcaccaccttcggtgtcggtgaattgtctgccttgaacggtattgccggttcttacgctgaacacgtcggtgtcttgcacgtcgtcggtgtcccatccatctcctctcaagctaagcaattgttgttgcaccacaccttgggtaacggtgacttcactgtcttccacagaatgtccgctaacatctctgagaccaccgctatggtcactgacatcgctaccgctccagctgagatcgacagatgtatcagaaccacctacatcacccaaagaccagtctacttgggtctaccagctaacttggtcgacctaaaggtcccagccaagcttttggaaaccccaattgacttgtccttgaagccaaacgacccagaagccgaaactgaagtcgttgacaccgtcttggaattgatcaaggctgctaagaacccagttatcttggctgatgcttgtgcttccagacacgacgtcaaggctgaaaccaagaagttgattgacgccactcaattcccatccttcgttaccccaatgggtaagggttccatcgacgaacaacacccaagattcggtggtgtctacgtcggtaccttgtccagaccagaagttaaggaagctgttgaatccgctgacttgatcttgtctgtcggtgctttgttgtccgatttcaacactggttctttctcttactcttacaagaccaagaacatcgtcgaattccactctgactacatcaagatcagaaacgctaccttcccaggtgtccaaatgaagttcgctttgcaaaagttgttgaacgccgtcccagaagctatcaagggttacaagccagtccctgtcccagctagagtcccagaaaacaagtcctgtgacccagctaccccattgaagcaagaatggatgtggaaccaagtttccaagttcttgcaagaaggtgatgttgttatcactgaaaccggtacctccgcttttggtatcaaccaaaccccattcccaaacaacgcttacggtatctcccaagttctatggggttccatcggtttcaccaccggtgcttgtttgggtgccgctttcgctgctgaagaaatcgacccaaagaagagagttatcttgttcattggtgacggttctttgcaattgactgtccaagaaatctccaccatgatcagatggggcttgaagccatacttgttcgtcttgaacaacgacggttacaccatcgaaagattgattcacggtgaaaaggctggttacaacgacatccaaaactgggaccacttggctctattgccaaccttcggtgctaaggactacgaaaaccacagagtcgccaccaccggtgaatgggacaagttgacccaagacaaggaattcaacaagaactccaagatcagaatgatcgaagttatgttgccagttatggacgctccaacttccttgattgaacaagctaagttgaccgcttccatcaacgctaagcaagaa

SEQ ID NO: 51 - protein, 564 aa, Candida glabrata
MSEITLGRYLFERLNQVDVKTIFGLPGDFNLSLLDKIYEVEGMRWAGNANELNAAYAADGYARIKGMSCIITTFGVGELSALNGIAGSYAEHVGVLHVVGVPSISSQAKQLLLHHTLGNGDFTVFHRMSANISETTAMVTDIATAPAEIDRCIRTTYITQRPVYLGLPANLVDLKVPAKLLETPIDLSLKPNDPEAETEVVDTVLELIKAAKNPVILADACASRHDVKAETKKLIDATQFPSFVTPMGKGSIDEQHPRFGGVYVGTLSRPEVKEAVESADLILSVGALLSDFNTGSFSYSYKTKNIVEFHSDYIKIRNATFPGVQMKFALQKLLNAVPEAIKGYKPVPVPARVPENKSCDPATPLKQEWMWNQVSKFLQEGDVVITETGTSAFGINQTPFPNNAYGISQVLWGSIGFTTGACLGAAFAAEEIDPKKRVILFIGDGSLQLTVQEISTMIRWGLKPYLFVLNNDGYTIERLIHGEKAGYNDIQNWDHLALLPTFGAKDYENHRVATTGEWDKLTQDKEFNKNSKIRMIEVMLPVMDAPTSLIEQAKLTASINAKQE

SEQ ID NO: 52 - DNA, 1788 bp, Pichia stipites
atggctgaagtctcattaggaagatatctcttcgagagattgtaccaattgcaagtgcagaccatcttcggtgtccctggtgatttcaacttgtcgcttttggacaagatctacgaagtggaagatgcccatggcaagaattcgtttagatgggctggtaatgccaacgaattgaatgcatcgtacgctgctgacggttactcgagagtcaagcgtttagggtgtttggtcactacctttggtgtcggtgaattgtctgctttgaatggtattgccggttcttatgccgaacatgttggtttgcttcatgtcgtaggtgttccatcgatttcctcgcaagctaagcaattgttacttcaccacactttgggtaatggtgatttcactgttttccatagaatgtccaacaacatttctcagaccacagcctttatctccgatatcaactcggctccagctgaaattgatagatgtatcagagaggcctacgtcaaacaaagaccagtttatatcgggttaccagctaacttagttgatttgaatgttccggcctctttgcttgagtctccaatcaacttgtcgttggaaaagaacgacccagaggctcaagatgaagtcattgactctgtcttagacttgatcaaaaagtcgctgaacccaatcatcttggtcgatgcctgtgcctcgagacatgactgtaaggctgaagttactcagttgattgaacaaacccaattcccagtatttgtcactccaatgggtaaaggtaccgttgatgagggtggtgtagacggagaattgttagaagatgatcctcatttgattgccaaggtcgctgctaggttgtctgctggcaagaacgctgcctctagattcggaggtgtttatgtcggaaccttgtcgaagcccgaagtcaaggacgctgtagagagtgcagatttgattttgtctgtcggtgcccttttgtctgatttcaacactggttcattttcctactcctacagaaccaagaacatcgtcgaattccattctgattacactaagattagacaagccactttcccaggtgtgcagatgaaggaagccttgcaagaattgaacaagaaagtttcatctgctgctagtcactatgaagtcaagcctgtgcccaagatcaagttggccaatacaccagccaccagagaagtcaagttaactcaggaatggttgtggaccagagtgtcttcgtggttcagagaaggtgatattattatcaccgaaaccggtacatcctccttcggtatagttcaatccagattcccaaacaacaccatcggtatctcccaagtattgtggggttctattggtttctctgttggtgccactttgggtgctgccatggctgcccaagaactcgaccctaacaagagaaccatcttgtttgttggagatggttctttgcaattgaccgttcaggaaatctccaccataatcagatggggtaccacaccttaccttttcgtgttgaacaatgacggttacaccatcgagcgtttgatccacggtgtaaatgcctcatataatgacatccaaccatggcaaaacttggaaatcttgcctactttctcggccaagaactacgacgctgtgagaatctccaacatcggagaagcagaagatatcttgaaagacaaggaattcggaaagaactccaagattagattgatagaagtcatgttaccaagattggatgcaccatctaaccttgccaaacaagctgccattacagctgccaccaacgccgaagct

SEQ ID NO: 53 - protein, 596 aa, Pichia stipites
MAEVSLGRYLFERLYQLQVQTIFGVPGDFNLSLLDKIYEVEDAHGKNSFRWAGNANELNASYAADGYSRVKRLGCLVTTFGVGELSALNGIAGSYAEHVGLLHVVGVPSISSQAKQLLLHHTLGNGDFTVFHRMSNNISQTTAFISDINSAPAEIDRCIREAYVKQRPVYIGLPANLVDLNVPASLLESPINLSLEKNDPEAQDEVIDSVLDLIKKSSNPIILVDACASRHDCKAEVTQLIEQTQFPVFVTPMGKGTVDEGGVDGELLEDDPHLIAKVAARLSAGKNAASRFGGVYVGTLSKPEVKDAVESADLILSVGALLSDFNTGSFSYSYRTKNIVEFHSDYTKIRQATFPGVQMKEALQELNKKVSSAASHYEVKPVPKIKLANTPATREVKLTQEWLWTRVSSWFREGDIIITETGTSSFGIVQSRFPNNTIGISQVLWGSIGFSVGATLGAAMAAQELDPNKRTILFVGDGSLQLTVQEISTIIRWGTTPYLFVLNNDGYTIERLIHGVNASYNDIQPWQNLEILPTFSAKNYDAVRISNIGEAEDILKDKEFGKNSKIRLIEVMLPRLDAPSNLAKQAAITAATNAEA

SEQ ID NO: 54 - DNA, 1707 bp, Pichia stipites
atggtatcaacctacccagaatcagaggttactctaggaaggtacctctttgagcgactccaccaattgaaagtggacaccattttcggcttgccgggtgacttcaacctttccttattggacaaagtgtatgaagttccggatatgaggtgggctggaaatgccaacgaattgaatgctgcctatgctgccgatggttactccagaataaagggattgtcttgcttggtcacaacttttggtgttggtgaattgtctgctttaaacggagttggtggtgcctatgctgaacacgtaggacttctacatgtcgttggagttccatccatatcgtcacaggctaaacagttgttgctccaccataccttgggtaatggtgacttcactgtttttcacagaatgtccaatagcatttctcaaactacagcatttctctcagatatctctattgcaccaggtcaaatagatagatgcatcagagaagcatatgttcatcagagaccagtttatgttggtttaccggcaaatatggttgatctcaaggttccttctagtctcttagaaactccaattgatttgaaattgaaacaaaatgatcctgaagctcaagaagttgttgaaacagtcctgaagttggtgtcccaagctacaaaccccattatcttggtagacgcttgtgccctcagacacaattgcaaagaggaagtcaaacaattggttgatgccactaattttcaagtctttacaactccaatgggtaaatctggtatctccgaatctcatccaagattgggcggtgtctatgtcgggacaatgtcgagtcctcaagtcaaaaaagccgttgaaaatgccgatcttatactatctgttggttcgttgttatcggacttcaatacaggttcattttcatactcctacaagacgaagaatgttgttgaattccactctgactatatgaaaatcagacaggccaccttcccaggagttcaaatgaaagaagccttgcaacagttgataaaaagggtctcttcttacatcaatccaagctacattcctactcgagttcctaaaaggaaacagccattgaaagctccatcagaagctcctttgacccaagaatatttgtggtctaaagtatccggctggtttagagagggtgatattatcgtaaccgaaactggtacatctgctttcggaattattcaatcccattttcccagcaacactatcggtatatcccaagtcttgtggggctcaattggtttcacagtaggtgcaacagttggtgctgccatggcagcccaggaaatcgaccctagcaggagagtaattttgttcgtcggtgatggttcattgcagttgacggttcaggaaatctctacgttgtgtaaatgggattgtaacaatacttatctttacgtgttgaacaatgatggttacactatagaaaggttgatccacggcaaaagtgccagctacaacgatatacagccttggaaccatttatccttgcttcgcttattcaatgctaagaaataccaaaatgtcagagtatcgactgctggagaattggactctttgttctctgataagaaatttgcttctccagataggataagaatgattgaggtgatgttatcgagattggatgcaccagcaaatcttgttgctcaagcaaagttgtctgaacgggtaaaccttgaaaat

SEQ ID NO: 55 - protein, 569 aa, Pichia stipites
MVSTYPESEVTLGRYLFERLHQLKVDTIFGLPGDFNLSLLDKVYEVPDMRWAGNANELNAAYAADGYSRIKGLSCLVTTFGVGELSALNGVGGAYAEHVGLLHVVGVPSISSQAKQLLLHHTLGNGDFTVFHRMSNSISQTTAFLSDISIAPGQIDRCIREAYVHQRPVYVGLPANMVDLKVPSSLLETPIDLKLKQNDPEAQEVVETVLKLVSQATNPIILVDACALRHNCKEEVKQLVDATNFQVFTTPMGKSGISESHPRLGGVYVGTMSSPQVKKAVENADLILSVGSLLSDFNTGSFSYSYKTKNVVEFHSDYMKIRQATFPGVQMKEALQQLIKRVSSYINPSYIPTRVPKRKQPLKAPSEAPLTQEYLWSKVSGWFREGDIIVTETGTSAFGIIQSHFPSNTIGISQVLWGSIGFTVGATVGAAMAAQEIDPSRRVILFVGDGSLQLTVQEISTLCKWDCNNTYLYVLNNDGYTIERLIHGKSASYNDIQPWNHLSLLRLFNAKKYQNVRVSTAGELDSLFSDKKFASPDRIRMIEVMLSRLDAPANLVAQAKLSERVNLEN

SEQ ID NO: 56 - DNA, 1692 bp, Kluyveromyces lactis
atgtctgaaattacattaggtcgttacttgttcgaaagattaaagcaagtcgaagttcaaaccatctttggtctaccaggtgatttcaacttgtccctattggacaatatctacgaagtcccaggtatgagatgggctggtaatgccaacgaattgaacgctgcttacgctgctgatggttacgccagattaaagggtatgtcctgtatcatcaccaccttcggtgtcggtgaattgtctgctttgaacggtattgccggttcttacgctgaacacgttggtgtcttgcacgttgtcggtgttccatccgtctcttctcaagctaagcaattgttgttgcaccacaccttgggtaacggtgacttcactgttttccacagaatgtgctccaacatttctgaaaccactgctatgatcaccgatatcaacactgccccagctgaaatcgacagatgtatcagaaccacttacgtttcccaaagaccagtctacttgggtttgccagctaacttggtcgacttgactgtcccagcttctttgttggacactccaattgatttgagcttgaagccaaatgacccagaagccgaagaagaagtcatcgaaaacgtcttgcaactgatcaaggaagctaagaacccagttatcttggctgatgcttgttgttccagacacgatgccaaggctgagaccaagaagttgatcgacttgactcaattcccagccttcgttaccccaatgggtaagggttccattgacgaaaagcacccaagattcggtggtgtctacgtcggtaccctatcttctccagctgtcaaggaagccgttgaatctgctcacttggttctatcggtcggtgctctattgtccgatttcaacactggttctttctcttactcttacaagaccaagaacattgtcgaattccactctgactacaccaagatcagaaggcctaccttcccaggtgtccaaatgaagttcgctttacaaaaattgttgactaaggttgccgatgctgctaagggttacaagccagttccagttccatctgaaccagaacacaacgaagatgtcgctgactccactccattgaagcaagaatgggtctggactcaagtcggtgaattcttgagagaaggtgatgttgttatcactgaaaccggtacctctgccttcggtatcaaccaaactcatttcccaaacaacacatacggtatctctcaagttttatggggttccattggtttcaccactggtgctaccttgggtgctgccttcgctgccgaagaaattgatccaaagaagagagttatcttattcattggtgacggttctttgcaattgactgttcaagaaatctccaccatgatcagatggggcttgaagccatacttgttcgtattgaacaacgacggttacaccattgaaagattgattcacggtgaaaccgctcaatacaactgtatccaaaactggcaacacttggaattattgccaactttcggtgccaaggactacgaagctgtcagagtttccaccactggtgaatggaacaagttgaccactgacgaaaagttccaagacaacaccagaatcagattgatcgaagttatgttgccaactatggatgctccatctaacttggttaagcaagctcaattgactgctgcatccaacgctaagaactaa

SEQ ID NO: 57 - protein, 563 aa, Kluyveromyces lactis
MSEITLGRYLFERLKQVEVQTIFGLPGDFNLSLLDNIYEVPGMRWAGNANELNAAYAADGYARLKGMSCIITTFGVGELSALNGIAGSYAEHVGVLHVVGVPSVSSQAKQLLLHHTLGNGDFTVFHRMSSNISETTAMITDINTAPAEIDRCIRTTYVSQRPVYLGLPANLVDLTVPASLLDTPIDLSLKPNDPEAEEEVIENVLQLIKEAKNPVILADACCSRHDAKAETKKLIDLTQFPAFVTPMGKGSIDEKHPRFGGVYVGTLSSPAVKEAVESADLVLSVGALLSDFNTGSFSYSYKTKNIVEFHSDYTKIRSATFPGVQMKFALQKLLTKVADAAKGYKPVPVPSEPEHNEAVADSTPLKQEWVWTQVGEFLREGDVVITETGTSAFGINQTHFPNNTYGISQVLWGSIGFTTGATLGAAFAAEEIDPKKRVILFIGDGSLQLTVQEISTMIRWGLKPYLFVLNNDGYTIERLIHGETAQYNCIQNWQHLELLPTFGAKDYEAVRVSTTGEWNKLTTDEKFQDNTRIRLIEVMLPTMDAPSNLVKQAQLTAATNAKN

SEQ ID NO: 58 - DNA, 1716 bp, Yarrowia lipolytica
atgagcgactccgaaccccaaatggtcgacctgggcgactatctctttgcccgattcaagcagctaggcgtggactccgtctttggagtgcccggcgacttcaacctcaccctgttggaccacgtgtacaatgtcgacatgcggtgggttgggaacacaaacgagctgaatgccggctactcggccgacggctactcccgggtcaagcggctggcatgtcttgtcaccacctttggcgtgggagagctgtctgccgtggctgctgtggcaggctcgtacgccgagcatgtgggcgtggtgcatgttgtgggcgttcccagcacctctgctgagaacaagcatctgctgctgcaccacacactcggtaacggcgacttccgggtctttgcccagatgtccaaactcatctccgagtacacccaccatattgaggaccccagcgaggctgccgacgtaatcgacaccgccatccgaatcgcctacacccaccagcggcccgtttacattgctgtgccctccaacttctccgaggtcgatattgccgaccaggctagactggatacccccctggacctttcgctgcagcccaacgaccccgagagccagtacgaggtgattgaggagatttgctcgcgtatcaaggccgccaagaagcccgtgattctcgtcgacgcctgcgcttcgcgatacagatgtgtggacgagaccaaggagctggccaagatcaccaactttgcctactttgtcactcccatgggtaagggttctgtggacgaggatactgaccggtacggaggaacatacgtcggatcgctgactgctcctgctactgccgaggtggttgagacagctgatctcatcatctccgtaggagctcttctgtcggacttcaacaccggttccttctcgtactcctactccaccaaaaacgtggtggaattgcattcggaccacgtcaaaatcaagtccgccacctacaacaacgtcggcatgaaaatgctgttcccgcccctgctcgaagccgtcaagaaactggttgccgagacccctgactttgcatccaaggctctggctgttcccgacaccactcccaagatccccgaggtacccgatgatcacattacgacccaggcatggctgtggcagcgtctcagttactttctgaggcccaccgacatcgtggtcaccgagaccggaacctcgtcctttggaatcatccagaccaagttcccccacaacgtccgaggtatctcgcaggtgctgtggggctctattggatactcggtgggagcagcctgtggagcctccattgctgcacaggagattgacccccagcagcgagtgattctgtttgtgggcgacggctctcttcagctgacggtgaccgagatctcgtgcatgatccgcaacaacgtcaagccgtacatttttgtgctcaacaacgacggctacaccatcgagaggctcattcacggcgaaaacgcctcgtacaacgatgtgcacatgtggaagtactccaagattctcgacacgttcaacgccaaggcccacgagtcgattgtggtcaacaccaagggcgagatggacgctctgttcgacaacgaagagtttgccaagcccgacaagatccggctcattgaggtcatgtgcgacaagatggacgcgcctgcctcgttgatcaagcaggctgagctctctgccaagaccaacgtttag

SEQ ID NO: 59 - protein, 571 aa, Yarrowia lipolytica
MSDSEPQMVDLGDYLFARFKQLGVDSVFGVPGDFNLTLLDHVYNVDMRWVGNTNELNAGYSADGYSRVKRLACLVTTFGVGELSAVAAVAGSYAEHVGVVHVVGVPSTSAENKHLLLHHTLGNGDFRVFAQMSKLISEYTHHIEDPSEAADVIDTAIRIAYTHQRPVYIAVPSNFSEVDIADQARLDTPLDLSLQPNDPESQYEVIEEICSRIKAAKKPVILVDACASRYRCVDETKELAKITNFAYFVTPMGKGSVDEDTDRYGGTYVGSLTAPATAEVVETADLIISVGALLSDFNTGSFSYSYSTKNVVELHSDHVKIKSATYNNVGMKMLFPPLLEAVKKLVAETPDFASKALAVPDTTPKIPEVPDDHITTQAWLWQRLSYFLRPTDIVVTETGTSSFGIIQTKFPHNVRGISQVLWGSIGYSVGAACGASIAAQEIDPQQRVILFVGDGSLQLTVTEISCMIRNNVKPYIFVLNNDGYTIERLIHGENASYNDVHMWKYSKILDTFNAKAHESIVVNTKGEMDALFDNEEFAKPDKIRLIEVMCDKMDAPASLIKQAELSAKTNV

SEQ ID NO: 60 - DNA, 1716 bp, Schizosaccharomyces pombe
atgagtggggatattttagtcggtgaatatctattcaaaaggcttgaacaattaggggtcaagtccattcttggtgttccaggagatttcaatttagctctacttgacttaattgagaaagttggagatgagaaatttcgttgggttggcaataccaatgagttgaatggtgcttatgccgctgatggttatgctcgtgttaatggtctttcagccattgttacaacgttcggcgtgggagagctttccgctattaatggagtggcaggttcttatgcggagcatgtcccagtagttcatattgttggaatgccttccacaaaggtgcaagatactggagctttgcttcatcatactttaggagatggagactttcgcactttcatggatatgtttaagaaagtttctgcctacagtataatgatcgataacggaaacgatgcagctgaaaagatcgatgaagccttgtcgatttgttataaaaaggctaggcctgtttacattggtattccttctgatgctggctacttcaaagcatcttcatcaaatcttgggaaaagactaaagctcgaggaggatactaacgatccagcagttgagcaagaagtcatcaatcatatctcggaaatggttgtcaatgcaaagaaaccagtgattttaattgacgcttgtgctgtaagacatcgtgtcgttccagaagtacatgagctgattaaattgacccatttccctacatatgtaactcccatgggtaaatctgcaattgacgaaacttcgcaattttttgacggcgtttatgttggttcaatttcagatcctgaagttaaagacagaattgaatccactgatctgttgctatccatcggtgctctcaaatcagactttaacacgggttccttctcttaccacctcagccaaaagaatgccgttgagtttcattcagaccacatgcgcattcgatatgctctttatccaaatgtagccatgaagtatattcttcgcaaactgttgaaagtacttgatgcttctatgtgtcattccaaggctgctcctaccattggctacaacatcaagcctaagcatgcggaaggatattcttccaacgagattactcattgctggttttggcctaaatttagtgaatttttgaagccccgagatgttttgatcaccgagactggaactgcaaactttggtgtccttgattgcaggtttccaaaggatgtaacagccatttcccaggtattatggggatctattggatactccgttggtgcaatgtttggtgctgttttggccgtccacgattctaaagagcccgatcgtcgtaccattcttgtagtaggtgatggatccttacaactgacgattacagagatttcaacctgcattcgccataacctcaaaccaattattttcataattaacaacgacggttacaccattgagcgtttaattcatggtttgcatgctagctataacgaaattaacactaaatggggctaccaacagattcccaagtttttcggagctgctgaaaaccacttccgcacttactgtgttaaaactcctactgacgttgaaaagttgtttagcgacaaggagtttgcaaatgcagatgtcattcaagtagttgagcttgtaatgcctatgttggatgcacctcgtgtcctagttgagcaagccaagttgacgtctaagatcaataagcaatga

SEQ ID NO: 61 - protein, 571 aa, Schizosaccharomyces pombe
MSGDILVGEYLFKRLEQLGVKSILGVPGDFNLALLDLIEKVGDEKFRWVGNTNELNGAYAADGYARVNGLSAIVTTFGVGELSAINGVAGSYAEHVPVVHIVGMPSTKVQDTGALLHHTLGDGDFRTFMDMFKKVSAYSIMIDNGNDAAEKIDEALSICYKKARPVYIGIPSDAGYFKASSSNLGKRLKLEEDTNDPAVEQEVINHISEMVVNAKKPVILIDACAVRHRVVPEVHELIKLTHFPTYVTPMGKSAIDETSQFFDGVYVGSISDPEVKDRIESTDLLLSIGALKSDFNTGSFSYHLSQKNAVEFHSDHMRIRYALYPNVAMKYILRKLLKVLDASMCHSKAAPTIGYNIKPKHAEGYSSNEITHCWFWPKFSEFLKPRDVLITETGTANFGVLDCRFPKDVTAISQVLWGSIGYSVGAMFGAVLAVHDSKEPDRRTILVVGDGSLQLTITEISTCIRHNLKPIIFIINNDGYTIERLIHGLHASYNEINTKWGYQQIPKFFGAAENHFRTYCVKTPTDVEKLFSDKEFANADVIQVVELVMPMLDAPRVLVEQAKLTSKINKQ

SEQ ID NO: 62 - DNA, 1689 bp, Zygosaccharomyces rouxii
atgtctgaaattactctaggtcgttacttgttcgaaagattaaagcaagttgacactaacaccatcttcggtgttccaggtgacttcaacttgtccttgttggacaaggtctacgaagtgcaaggtctaagatgggctggtaacgctaacgaattgaacgctgcctacgctgctgacggttacgccagagttaagggtttggctgctttgatcaccaccttcggtgtcggtgaattgtctgctttgaacggtattgcaggttcttacgctgaacacgttggtgttttgcacattgttggtgttccatctgtctcttctcaagctaagcaattgttgttgcaccacaccttgggtaacggtgacttcactgttttccacagaatgtccgccaacatctctgaaaccaccgctatgttgaccgacatcactgctgctccagctgaaattgaccgttgcatcagagttgcttacgtcaaccaaagaccagtctacttgggtctaccagctaacttggttgaccaaaaggtcccagcttctttgttgaacactccaattgatctatctctaaaggagaacgacccagaagctgaaaccgaagttgttgacaccgttttggaattgatcaaggaagctaagaacccagttatcttggctgatgcttgctgctccagacacgacgtcaaggctgaaaccaagaagttgatcgacttgactcaattcccatctttcgttactcctatgggtaagggttccatcgacgaacaaaacccaagattcggtggtgtctacgtcggtactctatccagcccagaagttaaggaagctgttgaatctgctgacttggttctatctgtcggtgctctattgtccgatttcaacactggttctttctcttactcttacaagaccaagaacgttgttgaattccactctgaccacatcaagatcagaaacgctaccttcccaggtgttcaaatgaaattcgttttgaagaaactattgcaagctgtcccagaagctgtcaagaactacaagccaggtccagtcccagctccgccatctccaaacgctgaagttgctgactctaccaccttgaagcaagaatggttatggagacaagtcggtagcttcttgagagaaggtgatgttgttattaccgaaactggtacctctgctttcggtatcaaccaaactcacttccctaaccaaacttacggtatctctcaagtcttgtggggttctattggttacaccactggttccactttgggtgctgccttcgctgctgaagaaattgaccctaagaagagagttatcttgttcattggtgacggttctctacaattgaccgttcaagaaatctccaccatgatcagatggggtctaaagccatacttgttcgttttgaacaacgatggttacaccattgaaagattgattcacggtgaaaccgctgaatacaactgtatccaaccatggaagcacttggaattgttgaacaccttcggtgccaaggactacgaaaaccacagagtctccactgtcggtgaatggaacaagttgactcaagatccaaaattcaacgaaaactctagaattagaatgatcgaagttatgcttgaagtcatggacgctccatcttctttggtcgctcaagctcaattgaccgctgctactaacgctaagcaa

SEQ ID NO: 63 - protein, 563 aa, Zygosaccharomyces rouxii
MSEITLGRYLFERLKQVDTNTIFGVPGDFNLSLLDKVYEVQGLRWAGNANELNAAYAADGYARVKGLAALITTFGVGELSALNGIAGSYAEHVGVLHIVGVPSVSSQAKQLLLHHTLGNGDFTVFHRMSANISETTAMLTDITAAPAEIDRCIRVAYVNQRPVYLGLPANLVDQKVPASLLNTPIDLSLKENDPEAETEVVDTVLELIKEAKNPVILADACCSRHDVKAETKKLIDLTQFPSFVTPMGKGSIDEQNPRFGGVYVGTLSSPEVKEAVESADLVLSVGALLSDFNTGSFSYSYKTKNVVEFHSDHIKIRNATFPGVQMKFVLKKLLQAVPEAVKNYKPGPVPAPPSPNAEVADSTTLKQEWLWRQVGSFLREGDVVITETGTSAFGINQTHFPNQTYGISQVLWGSIGYTTGSTLGAAFAAEEIDPKKRVILFIGDGSLQLTVQEISTMIRWGLKPYLFVLNNDGYTIERLIHGETAEYNCIQPWKHLELLNTFGAKDYENHRVSTVGEWNKLTQDPKFNENSRIRMIEVMLEVMDAPSSLVAQAQLTAATNAKQ

SEQ ID NO: 64 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 65 - protein, 343 aa, Anaerostipes caccae
MEECKMAKIYYQEDCNLSLLDGKTIAVIGYGSQGHAHALNAKESGCNVIIGLYEGAKEWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 66 - protein, 343 aa, Anaerostipes caccae
MEECKMAKIYYQEDCNLSLLDGKTIAVIGYGSQGHAHALNAKESGCNVIIGLYEGAKDWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 67 - protein, 338 aa, Pseudomonas fluorescens
MKVFYDKDCDLSIIQGKKVAIIGYGSQGHAQACNLKDSGVDVTVGLRKGSATVAKAEAHGLKVTDVAAAVAGADLVMILTPDEFQSQLYKNEIEPNIKKGATLAFSHGFAIHYNQVVPRADLDVIMIAPKAPGHTVRSEFVKGGGIPDLIAIYQDASGNAKNVALSYAAGVGGGRTGIIETTFKDETETDLFGEQAVLCGGTVELVKAGFETLVEAGYAPEMAYFECLHELKLIVDLMYEGGIANMNYSISNNAEYGEYVTGPEVINAESRQAMRNALKRIQDGEYAKMFISEGATGYPSMTAKRRNNAAHGIEIIGEQLRSMMPWIGANKIVDKAKN

SEQ ID NO: 68 - protein, 571 aa, Streptococcus mutans
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 69 - protein, 546 aa, Macrococcus caseolyticus
MKQRIGQYLIDALHVNGVDKIFGVPGDFTLAFLDDIIRHDNVEWVGNTNELNAAYAADGYARVNGLAAVSTTFGVGELSAVNGIAGSYAERVPVIKISGGPSSVAQQEGRYVHHSLGEGIFDSYSKMYAHITATTTILSVDNAVDEIDRVIHCALKEKRPVHIHLPIDVALTEIEIPHAPKVYTHESQNVDAYIQAVEKKLMSAKQPVIIAGHEINSFKLHEQLEQFVNQTNIPVAQLSLGKSAFNEENEHYLGIYDGKIAKENVREYVDNADVILNIGAKLTDSATAGFSYKFDTNNIIYINHNDFKAEDVISDNVSLIDLVNGLNSIDYRNETHYPSYQRSDMKYELNDAPLTQSNYFKMMNAFLEKDDILLAEQGTSFFGAYDLSLYKGNQFIGQPLWGSIGYTFPSLLGSQLADMHRRNILLIGDGSLQLTVQALSTMIRKDIKPIIFVINNDGYTVERLIHGMEEPYNDIQMWNYKQLPEVFGGKDTVKVHDAKTSNELKTVMDSVKADKDHMHFIEVHMAVEDAPKKLIDIAKAFSDANK

SEQ ID NO: 70 - protein, 548 aa, Listeria grayi
MYTVGQYLVDRLEEIGIDKVFGVPGDYNLTFLDYIQNHEGLSWQGNTNELNAAYAADGYARERGVSALVTTFGVGELSAINGTAGSFAEQVPVIHIVGSPTMNVQSNKKLVHHSLGMGNFHNFSEMAKEVTAATTMLTEENAASEIDRVLETALLEKRPVYINLPIDIAHKAIVKPAKALQTEKSSGEREAQLAEIILSHLEKAAQPIVIAGHEIARFQIRERFENWINQTKLPVTNLAYGKGSFNEENEHFIGTYYPAFSDKNVLDYVDNSDFVLHFGGKIIDNSTSSFSQGFKTENTLTAANDIIMLPDGSTYSGISLNGLLAELEKLNFTFADTAAKQAELAVFEPQAETPLKQDRFHQAVMNFLQADDVLVTEQGTSSFGLMLAPLKKGMNLISQTLWGSIGYTLPAMIGSQIAAPERRHILSIGDGSFQLTAQEMSTIFREKLTPVIFIINNDGYTVERAIHGEDESYNDIPTWNLQLVAETFGGDAETVDTHNVFTETDFANTLAAIDATPQKAHVVEVHMEQMDMPESLRQIGLALSKQNS

SEQ ID NO: 71 - protein, 348 aa, Achromobacter xylosoxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

SEQ ID NO: 72 - protein, 347 aa, Beijerickia indica
MKALVYRGPGQKLVEERQKPELKEPGDAIVKVTKTTICGTDLHILKGDVATCKPGRVLGHEGVGVIESVGSGVTAFQPGDRVLISCISSCGKCSFCRRGMFSHCTTGGWILGNEIDGTQAEYVRVPHADTSLYRIPAGADEEALVMLSDILPTGFECGVLNGKVAPGSSVAIVGAGPVGLAALLTAQFYSPAEIIMIDLDDNRLGLAKQFGATRTVNSTGGNAAAEVKALTEGLGVDTAIEAVGIPATFELCQNIVAPGGTIANVGVHGSKVDLHLESLWSHNVTITTRLVDTATTPMLLKTVQSHKLDPSRLITHRFSLDQILDAYETFGQAASTQALKVIISMEA

SEQ ID NO: 73 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 74 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 75 - DNA, 6525 bp, Artificial sequence
  pFBA1-413N
ccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggctcagtcaaagcacggtctaacaaaagaaatgacaatgaaataccgtatggaagggtgcgtcgatggacataaatttgtgatcacgggagagggcattggatatccgttcaaagggaaacaggctattaatctgtgtgtggtcgaaggtggaccattgccatttgccgaagacatattgtcagctgcctttatgtacggaaacagggttttcactgaatatcctcaagacatagctgactatttcaagaactcgtgtcctgctggttatacatgggacaggtcttttctctttgaggatggagcagtttgcatatgtaatgcagatataacagtgagtgttgaagaaaactgcatgtatcatgagtccaaattttatggagtgaattttcctgctgatggacctgtgatgaaaaagatgacagataactgggagccatcctgcgagaagatcataccagtacctaagcaggggatattgaaaggggatgtctccatgtacctccttctgaaggatggtgggcgtttacggtgccaattcgacacagtttacaaagcaaagtctgtgccaagaaagatgccggactggcacttcatccagcataagctcacccgtgaagaccgcagcgatgctaagaatcagaaatggcatctgacagaacatgctattgcatccggatctgcattgccctgagcggccgcctcgagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaatcgagagct

SEQ ID NO: 76 - DNA, 12298 bp, Artificial sequence
  pLH801L2V4
tcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcggccgcacctggtaaaacctctagtggagtagtagatgtaatcaatgaagcggaagccaaaagaccagagtagaggcctatagaagaaactgcgataccttttgtgatggctaaacaaacagacatctttttatatgtttttacttctgtatatcgtgaagtagtaagtgataagcgaatttggctaagaacgttgtaagtgaacaagggacctcttttgcctttcaaaaaaggattaaatggagttaatcattgagatttagttttcgttagattctgtatccctaaataactcccttacccgacgggaaggcacaaaagacttgaataatagcaaacggccagtagccaagaccaaataatactagagttaactgatggtcttaaacaggcattacgtggtgaactccaagaccaatatacaaaatatcgataagttattcttgcccaccaatttaaggagcctacatcaggacagtagtaccattcctcagagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcggaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaactgtttaaacagtatggaagaatgtaagatggctaagatttactaccaagaagactgtaacttgtccttgttggatggtaagactatcgccgttatcggttacggttctcaaggtcacgctcatgccctgaatgctaaggaatccggttgtaacgttatcattggtttatacgaaggtgcggaggagtggaaaagagctgaagaacaaggtttcgaagtctacaccgctgctgaagctgctaagaaggctgacatcattatgatcttgatcccagatgaaaagcaggctaccatgtacaaaaacgacatcgaaccaaacttggaagccggtaacatgttgatgttcgctcacggtttcaacatccatttcggttgtattgttccaccaaaggacgttgatgtcactatgatcgctccaaagggtccaggtcacaccgttagatccgaatacgaagaaggtaaaggtgtcccatgcttggttgctgtcgaacaagacgctactggcaaggctttggatatggctttggcctacgctttagccatcggtggtgctagagccggtgtcttggaaactaccttcagaaccgaaactgaaaccgacttgttcggtgaacaagctgttttatgtggtggtgtctgcgctttgatgcaggccggttttgaaaccttggttgaagccggttacgacccaagaaacgcttacttcgaatgtatccacgaaatgaagttgatcgttgacttgatctaccaatctggtttctccggtatgcgttactctatctccaacactgctgaatacggtgactacattaccggtccaaagatcattactgaagataccaagaaggctatgaagaagattttgtctgacattcaagatggtacctttgccaaggacttcttggttgacatgtctgatgctggttcccaggtccacttcaaggctatgagaaagttggcctccgaacacccagctgaagttgtcggtgaagaaattagatccttgtactcctggtccgacgaagacaagttgattaacaactgaggccctgcaggccagaggaaaataatatcaagtgctggaaactttttctcttggaatttttgcaacatcaagtcatagtcaattgaattgacccaatttcacatttaagattttttttttttcatccgacatacatctgtacactaggaagccctgtttttctgaagcagcttcaaatatatatattttttacatatttattatgattcaatgaacaatctaattaaatcgaaaacaagaaccgaaacgcgaataaataatttatttagatggtgacaagtgtataagtcctcatcgggacagctacgatttctctttcggttttggctgagctactggttgctgtgacgcagcggcattagcgcggcgttatgagctaccctcgtggcctgaaagatggcgggaataaagcggaactaaaaattactgactgagccatattgaggtcaatttgtcaactcgtcaagtcacgtttggtggacggcccctttccaacgaatcgtatatactaacatgcgcgcgcttcctatatacacatatacatatatatatatatatatatgtgtgcgtgtatgtgtacacctgtatttaatttccttactcgcgggtttttcttttttctcaattcttggcttcctctttctcgagcggaccggatcctcgcgaactccaaaatgagctatcaaaaacgatagatcgattaggatgactttgaaatgactccgcagtggactggccgttaatttcaagcgtgagtaaaatagtgcatgacaaaagatgagctaggcttttgtaaaaatatcttacgttgtaaaattttagaaatcattatttccttcatatcattttgtcattgaccttcagaagaaaagagccgaccaataatataaataaataaataaaaataatattccattatttctaaacagattcaatactcattaaaaaactatatcaattaatttgaattaacttaattaattattttttgccagtttcttcaggcttccaaaagtctgttacggctcccctagaagcagacgaaacgatgtgagcatatttaccaaggataccgcgtgaatagagcggtggcaattcaatggtctcttgacgatgttttaactcttcatcggagatatcaaagtgtaattccttagtgtcttggtcaatagtgactatgtctcctgtttgcaggtaggcgattggaccgccatcttgtgcttcaggagcgatatgacccacgacaagaccataagtaccacctgagaagcggccatctgtcagaagggcaactttttcaccttgccctttaccaacaatcattgatgaaagggaaagcatttcaggcataccaggaccgccctttggtcctacaaaacgtacgacaacaacatcaccatcaacaatatcatcattcaagacagcttcaatggcttcttcttcagaattaaagaccttagcaggaccgacatgacgacgcacttttacaccagaaactttggcaacggcaccgtctggagccaagttaccatggagaataatgaccggaccatcttcacgtttaggattttcaagcggcataataaccttttgaccaggtgttaaatcatcaaaagccttcaaattttcagcgactgttttgccagtacaagtgatacggtcaccatgaaggaagccatttttaaggagatatttcataactgctggtacccctccgaccttgtaaaggtcttggaatacatattgaccagaaggtttcaaatcagccaaatgaggaactttttcttggaaagtattgaaatcatcaagtgtcaattccacattagcagcatgggcaatagctaagaggtgaagggttgagttggttgaacctcccagagccatagttacagtaatagcatcttcaaaagcttcacgcgttaaaatgtcagaaggttttaagcccatttcgagcattttgacaacagcgcgaccagcttcttcaatatctgctttcttttctgcggattcagccgggtgagaagatgaacccggaaggctaagtcccaaaacttcaatagctgtcgccattgtgttagcagtatacataccaccgcagcctccaggaccgggacaagcattacattccaaagctttaacttcttctttggtcatatcgccgtggttccaatggccgacaccttcaaagacagagactaaatcgatatctttgccgtctaaattaccaggtgcaattgttccgccgtaagcaaaaatggctgggatatccatgttagccatagcgataacagaaccgggcatgtttttatcacaaccgccaatggctacaaaagcatccgcattatgacctcccatggctgcttcaatagaatctgcaataatatcacgagatgtcaaggagaaacgcattccttgggttcccatggcgattccatcagaaaccgtgattgttccgaactgaactggccaagcaccagcttccttaacaccgactttggctagtttaccaaagtcatgtaagtggatattacaaggtgtgttttcagcccaagttgaaatgacaccgacgataggtttttcaaagtcttcatcttgcataccagttgcacgcaacatagcacgattaggtgatttaaccattgaatcgtaaacagaactacgatttcttaagtctttaagagtttttttgtcagtcatactcacgtgaaacttagattagattgctatgctttctttccaatgagcaagaagtaaaaaaagttgtaatagaacaggaaaaatgaagctgaaacttgagaaattgaagaccgtttgttaactcaaatatcaatgggaggtcgtcgaaagagaacaaaatcgaaaaaaaagttttcaagagaaagaaacgtgataaaaatttttattgccttctccgacgaagaaaaagggacgaggcggtctctttttccttttccaaacctttagtacgggtaattaacggcaccctagaggaaggaggagggggaatttagtatgctgtgcttgggtgttttgaagtggtacggcggtgcgcggagtccgagaaaatctggaagagtaaaaaaggagtagagacattttgaagctatgccggcagatctatttaaatggcgcgccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaagcttgcatgcctgcaggtcgactctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcattttttttttttttttttttttttttttttttttttttttttttttttttttgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggctttaccttcttcaggcaagttcaatgacaatttcaacatcattgcagcagacaagatagtggcgatagggttgaccttattctttggcaaatctggagcggaaccatggcatggttcgtacaaaccaaatgcggtgttcttgtctggcaaagaggccaaggacgcagatggcaacaaacccaaggagcctgggataacggaggcttcatcggagatgatatcaccaaacatgttgctggtgattataataccatttaggtgggttgggttcttaactaggatcatggcggcagaatcaatcaattgatgttgaactttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctaacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaaaaaaaaaaaaaaaaaaaaaaaatgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagaggatc

SEQ ID NO: 77 - DNA, 4161 bp, Artificial sequence
  OLE1 ::Yld9d/LoxP/URA3 gene/LoxP DNA cassette
ggatgactctgccagcagtggcattgtcgacgaagtcgacttaacggaagcgcgcgcgtaatacgactcactatagggcgaattgggtaccgggccccccctcgaggtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggtcaaaaacgtagaccaagtagacttatcccaagtagacacaatcgcttcaggtagagatgtcaattacaaggtaaaatacaccagtggtgttaaaatgtctcaaggtgcatatgatgacaagggtagacatatttcagaacaaccttttacttgggccaattggcatcaacacatcaactggttgaacttcatattagttatcgctttgccattatcttcattcgctgcagccccttttgtatctttcaactggaaaacagctgcatttgccgttggttattacatgtgtaccggtttgggtattactgctggttatcatagaatgtgggctcacagagcatacaaagccgctttaccagtcagaattatattggccttattcggtggtggtgctgtagaaggttctattagatggtgggcttccagtcatagagttcatcacagatggactgattctaataaggatccttatgacgcaagaaagggtttttggttctcacactttggttggatgttgttagttccaaatcctaaaaacaagggtagaacagatatatcagacttgaataacgattgggttgtcagattgcaacataagtactacgtatacgttttggtctttatggctatcgtcttgccaaccttagtatgtggtttcggttggggtgactggaagggtggtttggtatatgctggtatcatgagatacacatttgttcaacaagtcaccttctgcgttaattctttagcacattggattggtgaacaaccatttgatgacagaagaacacctagagatcatgccttgactgctttagttacattcggtgaaggttatcacaattttcatcacgaattcccatccgattacagaaacgctttgatctggtaccaatacgaccctactaaatggttgatctggacattaaagcaagttggtttggcttgggatttgcaaacctttagtcaaaatgcaattgaacaaggtttggtccaacaaagacaaaagaaattggacaagtggagaaacaacttaaactggggtatcccaatagaacaattgcctgttatagaattcgaagaattccaagaacaagcaaagaccagagatttggttttaatttccggtatagtacatgacgttagtgcctttgtcgaacatcacccaggtggtaaagctttgattatgtccgcagttggtaaagatggtactgctgttttcaatggtggtgtctacagacattccaatgcaggtcacaacttgttagccaccatgagagtaagtgttattagaggtggtatggaagtcgaagtatggaagactgcacaaaacgaaaagaaagatcaaaacatcgtctctgacgaatcaggtaatagaattcatagagcaggtttacaagccacaagagtagaaaaccctggcatgtctggtatggcagcctaagcggccgcctcgagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaatcgagagctccagcttttgttccctttagtgagggttaattgcgcgcgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcactggccgtcgtcatccaggtggtgaaactttaattaaaactgcattaggtaaggacgc

SEQ ID NO: 78 - DNA, 6728 bp, Artificial sequence
  pJT254
tcgcgcgtttcggtgatgacggtgaaaacctctgacacatgcagctcccggagacggtcacagcttgtctgtaagcggatgccgggagcagacaagcccgtcagggcgcgtcagcgcgtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaatgtccaatttactgcccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgtggtgttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 79 - DNA, 3666 bp, Artificial sequence
  OLE1 Mad9d cassette
atcggctcctggctcatcgagtcttgcaaatcagcatatacatatatatatgggggcagatcttgattcatttattgttctatttccatctttcctacttctgtttccgtttatattttgtattacgtagaatagaacatcatagtaatagatagttgtggtgatcatattataaacagcactaaaacattacaacaaagatggcaacacctttacctccaacattcactgtcccagcctcctccaccgaaaccagaagagaccctttacctcacgacgtattacctccattgtttaatggtgaaaaggttaacatattgaacatatggaaatatttggattggaagcatgtcattggtttgttagttactcctttggtcgctttatacggcatgtgtactacagaattgcacaccaagactttagtatggtccatagtttactacttcgcaaccggtttgggtataactgccggttatcatagattatgggcacacagagcctacaacgctggtccagcaatgagttttgcattggccttattcggtgctggtgcagttgaaggttccattaaatggtggagtagaggtcatagagcacatcacagatggacagataccgaaaaggacccttattctgcacatagaggtgttttctattcacacttaggttggttgttaatcaaaagaccaggttggaagattggtcatgctgatgtagatgacttgaataagaaccctttagttcaatggcaacataagcactatttgatcttagttattttgatgggtttagtcttcccaactgccgtagctggtttgggttggggtgactggagaggtggttacttctacgctgcaatcttgagattgatcttcgttcatcacgctacattctgcgtcaattccttggcacactggttaggtgacggtccatttgatgacagacatacccctagagatcactttattactgccttcttgacattaggtgaaggttatcataactttcatcaccaattcccacaagactacagatctgcaatcagattctatcaatacgatcctacaaaatggttgattgccacctgtgctttctttggttttgcttcacatttgaagacattcccagaaaacgaaattaagaaaggtaaattgcaaatgatcgaaaaggaagttttggaaaagaaaactaagttgcaatggggtacaccaatagcagatttgcctatcttgtctttcgaagacttccaacatgcctgcaagaacgatagaaagcaatggatcttgttagaaggtgttgtctatgatgttgcagactttatgaccgaacacccaggtggtgaaaaatacattaagatgggtgttggtaaagacatgacttctgctttcaacggtggcatgtatgatcattccaatgccgctagaaacttgttaagtttgatgagagtcgccgtagttgaatttggtggtgaagtagaagctcaaaaatctagaccttcagtcacagtatacggtgaccattcaaaggaagaataagcggccgcctcgagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaatcgagagctccagcttttgttccctttagtgagggttaattgcgcgcgcattgcggattacgtattctaatgttcagtaccgttcgtataatgtatgctatacgaagttatgcagattgtactgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagataacttcgtataatgtatgctatacgaacggtaccagtgatgatacaacgagttagccaaggtgaattcgaaactctcccttttatgtttgcctaaagttctgaatatttcggtagcatccaaaagttgaactttattgtagatcttgtttagattgtaagctagaactggtaagttcaataaaaatacaaaccagtaaccgttcagtaagaatagtaatgacaaagcaccatgcaaggcggcttcaggggtaattaacttgttaactt

SEQ ID NO: 80 - DNA, 4120 bp, Artificial sequence
  FBA1Yld9d cassette
aaatccactatcgtctatcaactaatagttatattatcaatatattatcatatacggtgttaagatgatgacataagttatgagaagctgtcatcgaggttagaggccttaatggccgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggtcaaaaacgtagaccaagtagacttatcccaagtagacacaatcgcttcaggtagagatgtcaattacaaggtaaaatacaccagtggtgttaaaatgtctcaaggtgcatatgatgacaagggtagacatatttcagaacaaccttttacttgggccaattggcatcaacacatcaactggttgaacttcatattagttatcgctttgccattatcttcattcgctgcagccccttttgtatctttcaactggaaaacagctgcatttgccgttggttattacatgtgtaccggtttgggtattactgctggttatcatagaatgtgggctcacagagcatacaaagccgctttaccagtcagaattatattggccttattcggtggtggtgctgtagaaggttctattagatggtgggcttccagtcatagagttcatcacagatggactgattctaataaggatccttatgacgcaagaaagggtttttggttctcacactttggttggatgttgttagttccaaatcctaaaaacaagggtagaacagatatatcagacttgaataacgattgggttgtcagattgcaacataagtactacgtatacgttttggtctttatggctatcgtcttgccaaccttagtatgtggtttcggttggggtgactggaagggtggtttggtatatgctggtatcatgagatacacatttgttcaacaagtcaccttctgcgttaattctttagcacattggattggtgaacaaccatttgatgacagaagaacacctagagatcatgccttgactgctttagttacattcggtgaaggttatcacaattttcatcacgaattcccatccgattacagaaacgctttgatctggtaccaatacgaccctactaaatggttgatctggacattaaagcaagttggtttggcttgggatttgcaaacctttagtcaaaatgcaattgaacaaggtttggtccaacaaagacaaaagaaattggacaagtggagaaacaacttaaactggggtatcccaatagaacaattgcctgttatagaattcgaagaattccaagaacaagcaaagaccagagatttggttttaatttccggtatagtacatgacgttagtgcctttgtcgaacatcacccaggtggtaaagctttgattatgtccgcagttggtaaagatggtactgctgttttcaatggtggtgtctacagacattccaatgcaggtcacaacttgttagccaccatgagagtaagtgttattagaggtggtatggaagtcgaagtatggaagactgcacaaaacgaaaagaaagatcaaaacatcgtctctgacgaatcaggtaatagaattcatagagcaggtttacaagccacaagagtagaaaaccctggcatgtctggtatggcagcctaagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgctaccgttcgtataatgtatgctatacgaagttatgtatcgataagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttgatatcataacttcgtataatgtatgctatacgaacggtagcgcgccgaagctgaaacgcaaggattgataatgtaataggatcaatgaatataaacatataaaacggaatgaggaataatcgtaatattagtatgtagaaatatagattccattt

SEQ ID NO: 81 - DNA, 4009 bp, Artificial sequence
  FBA1Mad9d cassette
aaatccactatcgtctatcaactaatagttatattatcaatatattatcatatacggtgttaagatgatgacataagttatgagaagctgtcatcgaggttagaggccttaatggccgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggcaacacctttacctccaacattcactgtcccagcctcctccaccgaaaccagaagagaccctttacctcacgacgtattacctccattgtttaatggtgaaaaggttaacatattgaacatatggaaatatttggattggaagcatgtcattggtttgttagttactcctttggtcgctttatacggcatgtgtactacagaattgcacaccaagactttagtatggtccatagtttactacttcgcaaccggtttgggtataactgccggttatcatagattatgggcacacagagcctacaacgctggtccagcaatgagttttgcattggccttattcggtgctggtgcagttgaaggttccattaaatggtggagtagaggtcatagagcacatcacagatggacagataccgaaaaggacccttattctgcacatagaggtgttttctattcacacttaggttggttgttaatcaaaagaccaggttggaagattggtcatgctgatgtagatgacttgaataagaaccctttagttcaatggcaacataagcactatttgatcttagttattttgatgggtttagtcttcccaactgccgtagctggtttgggttggggtgactggagaggtggttacttctacgctgcaatcttgagattgatcttcgttcatcacgctacattctgcgtcaattccttggcacactggttaggtgacggtccatttgatgacagacatacccctagagatcactttattactgccttcttgacattaggtgaaggttatcataactttcatcaccaattcccacaagactacagatctgcaatcagattctatcaatacgatcctacaaaatggttgattgccacctgtgctttctttggttttgcttcacatttgaagacattcccagaaaacgaaattaagaaaggtaaattgcaaatgatcgaaaaggaagttttggaaaagaaaactaagttgcaatggggtacaccaatagcagatttgcctatcttgtctttcgaagacttccaacatgcctgcaagaacgatagaaagcaatggatcttgttagaaggtgttgtctatgatgttgcagactttatgaccgaacacccaggtggtgaaaaatacattaagatgggtgttggtaaagacatgacttctgctttcaacggtggcatgtatgatcattccaatgccgctagaaacttgttaagtttgatgagagtcgccgtagttgaatttggtggtgaagtagaagctcaaaaatctagaccttcagtcacagtatacggtgaccattcaaaggaagaataagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgctaccgttcgtataatgtatgctatacgaagttatgtatcgataagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttgatatcataacttcgtataatgtatgctatacgaacggtagcgcgccgaagctgaaacgcaaggattgataatgtaataggatcaatgaatataaacatataaaacggaatgaggaataatcgtaatattagtatgtagaaatatagattccattt

SEQ ID NO: 82 - DNA, 3499 bp, Artificial sequence
  FBA1Maelo cassette
aaatccactatcgtctatcaactaatagttatattatcaatatattatcatatacggtgttaagatgatgacataagttatgagaagctgtcatcgaggttagaggccttaatggccgtcgacgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaactagtgccaccatggaatctggtcctatgcctgctggtatcccttttcctgaatactacgacttctttatggattggaaaacacctttggctatcgctgccacttatacagctgcagttggtttattcaatccaaaggttggtaaagtttctagagttgtcgccaaatcagctaacgcaaagcctgctgaaagaactcaatctggtgccgctatgacagccttcgtcttcgtacataatttgatattgtgtgtttactcaggtatcacattctactacatgttcccagcaatggtcaaaaactttagaacccatactttacacgaagcatattgcgataccgaccaatctttatggaataacgccttgggttattggggttatttgttttatttgtcaaagttctacgaagttattgatactattataatcattttgaagggtagaagatcttcattgttacaaacctaccatcacgccggtgctatgataactatgtggtccggtatcaattatcaagctacaccaatctggatcttcgtagttttcaacagttttatccatacaatcatgtactgttactacgcattcacctccataggttttcacccacctggtaaaaagtatttgacaagtatgcaaataacccaattcttggttggtattaccatagctgtctcctatttgtttgtaccaggttgcatcagaactcctggtgcacaaatggccgtatggataaacgttggttacttgttccctttgacttatttgttcgttgacttcgctaaaagaacatactccaagagaagtgctattgcagcccaaaagaaagcacaataagcggccgcgttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgctaccgttcgtataatgtatgctatacgaagttatgtatcgataagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttgatatcataacttcgtataatgtatgctatacgaacggtagcgcgccgaagctgaaacgcaaggattgataatgtaataggatcaatgaatataaacatataaaacggaatgaggaataatcgtaatattagtatgtagaaatatagattccattt

(19) United States
(12) Patent Application Publication
(10) Pub. No.: US 2016/0326551 A1
(43) Pub. Date: Nov. 10, 2016

(54) ISOBUTANOL TOLERANCE IN YEAST WITH AN ALTERED LIPID PROFILE

(71) Applicant: BUTAMAX ADVANCED BIOFUELS LLC, Wilmington, DE (US)

(72) Inventors: Tina K. VAN DYK, Wilmington, DE (US); Zhixiong XUE, Chadds Ford, PA (US); Narendra S. YADAV, Wilmington, DE (US); Hongxiang ZHANG, Chadds Ford, PA (US)

(21) Appl. No.: 15/108,845

(22) PCT Filed: Dec. 22, 2014
(86) PCT No.: PCT/US14/71924

§ 371 Date: Jun. 29, 2016

Related U.S. Application Data
(60) Provisional application No. 61/922,346, filed on Dec. 31, 2013.

(51) Int. Cl. C12P 7/16; C10L 1/02; C12N 9/02; C12N 9/10; C12N 9/88; C12N 9/04

(52) U.S. Cl. CPC C12P 7/16; C12N 9/88; C12N 9/0006; C12N 9/0071; C12N 9/1007; C12N 9/1022; C12Y 114/19001; C12Y 114/19006; C12Y 201/01079; C12Y 202/01006; C12Y 101/01086; C12Y 401/01009; C12Y 401/01072; C10L 1/02; C10L 2290/26; C10L 2200/0469

(57) ABSTRACT

Provided herein are recombinant yeast host cells and methods for their use for production of fermentation products from an engineered pyruvate utilizing pathway. The yeast host cells provided herein comprise an altered lipid profile, which confers resistance to butanol.

### CROSS REFERENCE TO RELATED APPLICATION

This application claims benefit of priority from U.S. Provisional Application No. 61/922,346, filed Dec. 31, 2013, which is hereby incorporated by reference in its entirety.

### REFERENCE TO SEQUENCE LISTING SUBMITTED ELECTRONICALLY

The content of the electronically submitted sequence listing in ASCII text file (Name: 20141210_CL6046WOPCT_SequenceListing_ascii.txt, Size: 298,393 bytes, and Date of Creation: Dec. 10, 2014) filed with the application is incorporated herein by reference in its entirety.

### FIELD OF THE INVENTION

The invention relates to the fields of microbiology, fermentation, and genetic engineering. More specifically, yeast with altered lipid profiles are provided. Such yeast may be useful for production via engineered biosynthetic pathways.

### BACKGROUND OF THE INVENTION

Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

Butanol may be made through chemical synthesis or by fermentation. Isobutanol is a component of “fusel oil”, which can form under certain conditions as a result of incomplete metabolism of amino acids by yeast. Under some circumstances, isobutanol may be produced from catabolism of L-valine. (See, e.g., Dickinson et al., J. Biol. Chem. 273(40):25752-25756 (1998)). Additionally, recombinant microbial production hosts, expressing an isobutanol biosynthetic pathway have been described. (Donaldson et al., commonly owned U.S. Pat. Nos. 7,851,188 and 7,993,889).

Efficient biological production of butanols may be limited by butanol toxicity to the host microorganism used in fermentation for butanol production. Accordingly, there is a need for modifications that confer tolerance to butanol.

### SUMMARY OF THE INVENTION

Provided herein are recombinant yeast cells comprising an engineered pyruvate utilizing biosynthetic pathway and further comprising a cell membrane with an altered lipid profile. In some embodiments the recombinant yeast cell has an increased tolerance to butanol as compared to a recombinant yeast cell that does not comprise an altered lipid profile.

In some embodiments the altered lipid profile comprises an increase in the concentration of C18:1, C18:2, and C18:3 fatty acids as compared to a microorganism that lacks the cell membrane with an altered lipid profile. In some embodiments the altered lipid profile comprises an increase in the ratio of unsaturated to saturated fatty acids as compared to a microorganism that lacks the cell membrane with an altered lipid profile. In some embodiments the altered lipid profile comprises an increase in the concentration of cyclopropane fatty acid as compared to a microorganism that lacks the cell membrane with an altered lipid profile. In some embodiments the altered lipid profile comprises an increase in the C18 to C16 fatty acid concentration ratio as compared to a microorganism that does not comprise an altered lipid profile.

In some embodiments the microorganism is engineered to express a gene encoding a fatty acid desaturase. In a further embodiment the microorganism comprises a recombinantly expressed fatty acid desaturase enzyme selected from: (a) fatty acid desaturase having the EC number 1.14.19.1; (b) fatty acid desaturase having the EC number 1.14.19.6; (c) a polypeptide that has at least 90% identity to any one or more of SEQ ID NOs: 1, 9, or 2; (d) a polypeptide encoded by a nucleic acid sequence that has at least 90% identity to any one or more of SEQ ID NOs: 3, 10, or 4; (e) a polypeptide encoded by a nucleic acid sequence that is complementary to any one or more of SEQ ID NOs: 3, 10, or 4; (f) a polypeptide encoded by a nucleic acid sequence that hybridizes under stringent conditions any one or more of SEQ ID NOs: 3, 10, or 4; or (g) any two or more of (a), (b), (c), (d), (e), or (f).

In some embodiments the microorganism is engineered to express a gene encoding a cyclopropane fatty acid synthase enzyme. In a further embodiment the microorganism comprises a recombinantly expressed cyclopropane fatty acid synthase enzyme selected from: (a) a cyclopropane fatty acid synthase having the EC number 2.1.1.79; (b) a polypeptide that has at least 90% identity to any one or more of SEQ ID NOs: 5 or 6; (c) a polypeptide encoded by a nucleic acid sequence that has at least 90% identity to any one or more of SEQ ID NOs: 7 or 8; (d) a polypeptide encoded by a nucleic acid sequence that is complementary to any one or more of SEQ ID NOs: 7 or 8; (e) a polypeptide encoded by a nucleic acid sequence that hybridizes under stringent conditions any one or more of SEQ ID NOs: 7 or 8; or (f) any two or more of (a), (b), (c), (d) or (e).

In some embodiments the microorganism is engineered to express a fatty acid elongase enzyme. In a further embodiment the microorganism comprises a recombinantly expressed fatty acid elongase enzyme selected from: (a) a polypeptide that has at least 90% identity to any one or more of SEQ ID NOs: 15, 16, or 11; (b) a polypeptide encoded by a nucleic acid sequence that has at least 90% identity to any one or more of SEQ ID NOs: 17, 18, 12; (c) a polypeptide encoded by a nucleic acid sequence that is complementary to any one or more of SEQ ID NOs: 17, 18, 12; (d) a polypeptide encoded by a nucleic acid sequence that hybridizes under stringent conditions any one or more of SEQ ID NOs: 17, 18, 12; or (e) any two or more of (a), (b), (c), or (d).

In some embodiments the microorganism produces more butanol as compared to a microorganism that lacks the altered lipid profile. In some embodiments the microorganism further comprises at least one genetic modification in an endogenous pyruvate decarboxylase gene. In a further embodiment the pyruvate decarboxylase gene is PDC1, PDC5, PDC6, or combinations thereof. In some embodiments the microorganism comprises a genetic modification in an endogenous glycerol-3-phosphate dehydrogenase (GPD) genes. In a further embodiment the GPD gene is GPD2. In some embodiments the microorganism comprises a genetic modification in FRA2.

In some embodiments the pyruvate utilizing biosynthetic pathway is an engineered C3-C6 alcohol production pathway. In some embodiments the C3-C6 alcohol is selected from propanol, butanol, pentanol, or hexanol. In some embodiments the C3-C6 alcohol is butanol. In some embodiments the butanol is isobutanol. In some embodiments the engineered pathway comprises the following substrate to product conversions: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde; and (e) isobutyraldehyde to isobutanol; and wherein (i) the substrate to product conversion of step (a) is performed by a recombinantly expressed acetolactate synthase enzyme; (ii) the substrate to product conversion of step (b) is performed by a recombinantly expressed acetohydroxy acid isomeroreductase enzyme; (iii) the substrate to product conversion of step (c) is performed by a recombinantly expressed acetohydroxy acid dehydratase enzyme; (iv) the substrate to product conversion of step (d) is performed by a recombinantly expressed decarboxylase enzyme; and (v) the substrate to product conversion of step (e) is performed by an alcohol dehydrogenase enzyme; whereby isobutanol is produced from pyruvate via the substrate to product conversions of steps (a)-(e).

In some embodiments the microorganism comprises a recombinantly expressed acetolactate synthase enzyme selected from: (a) an acetolactate synthase having the EC number 2.2.1.6; (b) a polypeptide that has at least 90% identity to any one or more of SEQ ID NO:13, SEQ ID NO:14, or SEQ ID NO:19; (c) a polypeptide encoded by a nucleic acid sequence that has at least 90% identity to any one or more of SEQ ID NOs: 20, 21, or 22; (d) a polypeptide encoded by a nucleic acid sequence that is complementary to any one or more of SEQ ID NOs: 20, 21 or 22; (e) a polypeptide encoded by a nucleic acid sequence that hybridizes under stringent conditions any one or more of SEQ ID NOs: 20, 21, or 22; or (f) any two or more of (a), (b), (c), (d) or (e).

In some embodiments the microorganism comprises a recombinantly expressed acetohydroxy acid isomeroreductase enzyme selected from: (a) an acetohydroxy acid isomeroreductase having the EC number 1.1.1.86; (b) an acetohydroxy acid isomeroreductase that matches the KARI Profile HMI with an E value of <10⁻³ using hmmsearch; (c) a polypeptide that has at least 90% identity to any one or more of SEQ ID NOs: 23, 24, or 25; (d) a polypeptide encoded by a nucleic acid sequence that has at least 90% identity to any one or more of SEQ ID NOs: 26, 27, 28 or 29; (e) a polypeptide encoded by a nucleic acid sequence that is complementary to any one or more of SEQ ID NOs: 26, 27, 28 or 29; (f) is a polypeptide encoded by a nucleic acid sequence that hybridizes under stringent conditions any one or more of SEQ ID NOs: 26, 27, 28 or 29; or (g) any two or more of (a), (b), (c), (d), (e) or (f).

In some embodiments the microorganism comprises a recombinantly expressed acetohydroxy acid dehydratase enzyme selected from: (a) an acetohydroxy acid dehydratase having the EC number 4.2.1.9; (b) a polypeptide that has at least 90% identity to any one or more of SEQ ID NO: 30; SEQ ID NO: 31, SEQ ID NO: 32 or SEQ ID NO: 33; (c) a polypeptide encoded by a nucleic acid sequence that has at least 90% identity to any one or more of SEQ ID NOs: 34, 35, 36, or 37; (d) a polypeptide encoded by a nucleic acid sequence that is complementary to any one or more of SEQ ID NOs: 34, 35, 36, or 37; (e) a polypeptide encoded by a nucleic acid sequence that hybridizes under stringent conditions any one or more of SEQ ID NOs: 34, 35, 36, or 37; or (f) any two or more of (a), (b), (c), (d) or (e).

In some embodiments the microorganism comprises a decarboxylase enzyme selected from: (a) an α-keto acid decarboxylase having the EC number 4.1.1.72; (b) a pyruvate decarboxylase having the EC number 4.1.1.1; (c) a polypeptide that has at least 90% identity to SEQ ID NO: 38; SEQ ID NO: 39, or both; (d) a polypeptide encoded by a nucleic acid sequence that has at least 90% identity to any one or more of SEQ ID NOs: 40, 41, or 42; (e) is a polypeptide encoded by a nucleic acid sequence that is complementary to any one or more of SEQ ID NOs: 40, 41, or 42; (f) is a polypeptide encoded by a nucleic acid sequence that hybridizes under stringent conditions any one or more of SEQ ID: 40, 41, or 42; or (g) any two or more of (a), (b), (c), (d), (e) or (f).

In some embodiments the yeast is a member of the genus selected from Saccharomyces, Schizosaccharomyces, Hansenula, Candida, Kluyveromyces, Yarrowia, Issatchenkia, or Pichia. In some embodiments the yeast is selected from Saccharomyces cerevisiae, Schizosaccharomyces pombe, Kluyveromyces lactis, Kluyveromyces thermotolerans, Kluyveromyces marxianus, Candida glabrata, Candida albicans, Pichia stipitis, or Yarrowia lipolytica. In some embodiments the yeast is Saccharomyces cerevisiae.

Also provided herein is a method of producing a fermentation product from an engineered pyruvate biosynthetic pathway comprising providing the recombinant yeast described herein and growing the yeast under conditions whereby the fermentation product is produced from pyruvate. In some embodiments the fermentation product is a C3-C6 alcohol. In some embodiments the C3-C6 alcohol is selected from propanol, butanol, pentanol, or hexanol. In some embodiments the C3-C6 alcohol is butanol. In some embodiments the butanol is isobutanol.

In some embodiments the method comprises providing a yeast comprising an engineered isobutanol production pathway. In some embodiments the method comprises providing a yeast comprising a recombinantly expressed acetolactate synthase enzyme as described herein. In some embodiments the method comprises providing a yeast comprising a recombinantly expressed acetohydroxy acid isomeroreductase enzyme as described herein. In some embodiments the method comprises providing a yeast comprising a recombinantly expressed acetohydroxy acid dehydratase enzyme as described herein. In some embodiments the method comprises providing a yeast comprising a decarboxylase enzyme as described herein.

In some embodiments the butanol is recovered from the fermentation medium. In some embodiments the butanol is recovered by distillation, liquid-liquid extraction, extraction, adsorption, decantation, pervaporation, or combinations thereof. In some embodiments solids are removed from the fermentation medium. In some embodiments the solids are removed by centrifugation, filtration, or decantation. In some embodiments the solids are removed before recovering the butanol. In some embodiments the fermentation product is produced by batch, fed-batch, or continuous fermentation.

Also provided herein is a method of making a bio-based fuel comprising using a C3-C6 alcohol, produced by the methods provided herein, as a component of a bio-based fuel. In some embodiments the C3-C6 alcohol is selected from propanol, butanol, pentanol, or hexanol. In some embodiments the C3-C6 alcohol is butanol. In some embodiments the butanol is isobutanol.

Also provided herein is a bio-based fuel comprising a C3-C6 alcohol produced by the methods provided herein. In some embodiments the C3-C6 alcohol is selected from propanol, butanol, pentanol, or hexanol. In some embodiments the C3-C6 alcohol is butanol. In some embodiments the butanol is isobutanol.

Also provided herein is a method for improving production of a butanol comprising: (a) providing a recombinant yeast microorganism comprising an engineered butanol biosynthetic pathway selected from: (i) a 1-butanol pathway; (ii) a 2-butanol pathway; or (iii) an isobutanol biosynthetic pathway; and wherein the yeast microorganism of (a) also comprises a gene encoding a one or more of the following: (i) a polypeptide that has at least 90% identity to any one or more of SEQ ID NOs: 1, 9, or 2; (ii) a polypeptide that has at least 90% identity to any one or more of SEQ ID NOs: 5 or 6; or (iii) a polypeptide that has at least 90% identity to any one or more of SEQ ID NOs: 15, 16, or 11; and (b) contacting the yeast microorganism with fermentable sugar whereby the microorganism produces butanol and wherein the microorganism has improved tolerance to the butanol as compared to a yeast microorganism without an altered lipid profile.

Also provided herein is a method for producing a recombinant yeast microorganism having increased tolerance to a butanol comprising: (a) providing a recombinant yeast microorganism comprising an engineered butanol biosynthetic pathway selected from the group consisting of: (i) a 1-butanol pathway; (ii) a 2-butanol pathway; or (iii) an isobutanol biosynthetic pathway; and engineering the yeast microorganism of (a) to recombinantly express gene encoding one or more of: (i) a polypeptide that has at least 90% identity to any one or more of SEQ ID NOs: 1, 9, or 2; (ii) a polypeptide that has at least 90% identity to any one or more of SEQ ID NOs: 5 or 6; or (iii) a polypeptide that has at least 90% identity to any one or more of SEQ ID NOs: 15, 16, or 11.

Also provided herein is a method for improving fermentative production of a butanol comprising: (a) providing a recombinant yeast microorganism comprising an engineered butanol biosynthetic pathway selected from: (i) a 1-butanol pathway; (ii) a 2-butanol pathway; or (iii) an isobutanol biosynthetic pathway; and (b) contacting the yeast microorganism with fermentable sugar whereby the microorganism produces butanol; (c) contacting the yeast microorganism with fatty acids derived from biomass at a step in the fermentation process; wherein the microorganism has improved tolerance to the butanol as compared to a yeast microorganism not contacted with fatty acids derived from biomass at a step in the fermentation process; and wherein the microorganism has a cell membrane with an altered lipid profile as compared to a yeast microorganism not contacted with fatty acids derived from biomass at a step in the fermentation process. In a further embodiment the yeast microorganism is engineered to express a gene encoding one or more of: (a) a polypeptide that has at least 90% identity to any one or more of SEQ ID NOs: 1, 9, or 2; (b) a polypeptide that has at least 90% identity to any one or more of SEQ ID NOs: 5 or 6; or (c) a polypeptide that has at least 90% identity to any one or more of SEQ ID NOs: 15, 16, or 11.

Also provided herein is a method for altering the lipid profile of a yeast microorganism comprising contacting the microorganism with fatty acids derived from biomass. In some embodiments the method comprises contacting the microorganism with COFA. In some embodiments the method comprises contacting the microorganism with a fermentable carbon substrate in a fermentation medium under conditions whereby a fermentation product is produced. In some embodiments the microorganism comprises an engineered pyruvate utilizing biosynthetic pathway. In some embodiments the engineered pyruvate utilizing biosynthetic pathway is an engineered C3-C6 alcohol production pathway. In a further embodiment the C3-C6 alcohol is selected from propanol, butanol, pentanol, or hexanol. In a further embodiment the C3-C6 alcohol is butanol. In a further embodiment the butanol is isobutanol. In some embodiments the microorganism further comprises a gene encoding a one or more of the following: (i) a polypeptide that has at least 90% identity to any one or more of SEQ ID NOs: 1, 9, or 2; (ii) a polypeptide that has at least 90% identity to any one or more of SEQ ID NOs: 5 or 6; or (iii) a polypeptide that has at least 90% identity to any one or more of SEQ ID NOs: 15, 16, or 11.

### BRIEF DESCRIPTION OF THE DRAWINGS

The various embodiments of the invention can be more fully understood from the following detailed description and the accompanying sequence descriptions, which form a part of this application.

FIG. 1 depicts different isobutanol biosynthetic pathways. The steps labeled “a”, “b”, “c”, “d”, “e”, “f”, “g”, “h”, “i”, “j”, and “k” represent substrate to product conversions described below. “a” may be catalyzed, for example, by acetolactate synthase. “b” may be catalyzed, for example, by acetohydroxyacid reductoisomerase. “c” may be catalyzed, for example, by acetohydroxy acid dehydratase. “d” may be catalyzed, for example, by branched-chain keto acid decarboxylase. “e” may be catalyzed, for example, by branched chain alcohol dehydrogenase. “f” may be catalyzed, for example, by branched chain keto acid dehydrogenase. “g” may be catalyzed, for example, by acetylating aldehyde dehydrogenase. “h” may be catalyzed, for example, by transaminase or valine dehydrogenase. “i” may be catalyzed, for example, by valine decarboxylase. “j” may be catalyzed, for example, by omega transaminase. “k” may be catalyzed, for example by isobutyryl-CoA mutase.

### DETAILED DESCRIPTION

The present invention relates to recombinant yeast cells that are engineered for the production of a fermentation product that is synthesized from an engineered pyruvate utilizing biosynthetic pathway and that additionally comprise a cell membrane with an altered lipid profile. These yeast cells have increased tolerance to butanol, and they can be used for the production of C3-C6 alcohols, such as butanol, which are valuable as fuel additives to reduce demand for fossil fuels.

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

In order to further define this invention, the following terms and definitions are herein provided.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “consists of” or variations such as “consist of” or “consisting of,” as used throughout the specification and claims, indicate the inclusion of any recited integer or group of integers, but that no additional integer or group of integers may be added to the specified method, structure, or composition.

As used herein, the term “consists essentially of,” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. §2111.03.

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

In some instances, “biomass” as used herein refers to the cell biomass of the fermentation product-producing microorganism.

The term “bio-based fuel” as used herein refers to a fuel in which the carbon contained within the fuel is derived from recently living biomass. “Recently living biomass” are defined as organic materials having a ¹⁴C/¹²C isotope ratio in the range of from 1:0 to greater than 0:1 in contrast to a fossil-based material which has a ¹⁴C/¹²C isotope ratio of 0.1. The ¹⁴C/¹²C isotope ratio can be measured using methods known in the art such as the ASTM test method D 6866-05 (Determining the Biobased Content of Natural Range Materials Using Radiocarbon and Isotope Ratio Mass Spectrometry Analysis). A bio-based fuel is a fuel in its own right, but may be blended with petroleum-derived fuels to generate a fuel. A bio-based fuel may be used as a replacement for petrochemically-derived gasoline, diesel fuel, or jet fuel.

The term “fermentation product” includes any desired product of interest, including, but not limited to lactic acid, 3-hydroxy-propionic acid, acrylic acid, acetic acid, succinic acid, citric acid, fumaric acid, malic acid, itaconic acid, 1,3-propane-diol, ethylene, glycerol, isobutyrate, butanol and other lower alkyl alcohols, etc.

The term “lower alkyl alcohol” refers to any straight-chain or branched, saturated or unsaturated, alcohol molecule with 1-10 carbon atoms.

The term “C3-C6 alcohol” refers to any alcohol with 3-6 carbon atoms.

The term “pyruvate utilizing biosynthetic pathway” refers to any enzyme pathway that utilizes pyruvate as its starting substrate.

The term “butanol” refers to 1-butanol, 2-butanol, 2-butanone, isobutanol, or mixtures thereof. Isobutanol is also known as 2-methyl-1-propanol.

The term “engineered” as used herein refers to an enzyme pathway that is not present endogenously in a microorganism and is deliberately constructed to produce a fermentation product from a starting substrate through a series of specific substrate to product conversions.

The term “C3-C6 alcohol pathway” as used herein refers to an enzyme pathway to produce C3-C6 alcohols. For example, engineered isopropanol biosynthetic pathways are disclosed in U.S. Patent Appl. Pub. No. 2008/0293125, which is incorporated herein by reference. From time to time “C3-C6 alcohol pathway” is used synonymously with “C3-C6 alcohol production pathway”.

The term “butanol biosynthetic pathway” as used herein refers to an enzyme pathway to produce 1-butanol, 2-butanol, 2-butanone or isobutanol. For example, engineered isobutanol biosynthetic pathways are disclosed in U.S. Pat. Nos. 7,851,188 and 7,993,889, which are incorporated by reference herein. Additionally, an example of an engineered 1-butanol pathway is disclosed in U.S. Patent Appl. Pub. No. 2008/0182308, which is incorporated by reference herein. Examples of engineered 2-butanol and 2-butanone biosynthetic pathways are disclosed in U.S. Pat. No. 8,206,970 and U.S. Patent Pub. No. 2009/0155870, which are incorporated by reference herein. From time to time “butanol biosynthetic pathway” is used synonymously with “butanol production pathway”.

The term “isobutanol biosynthetic pathway” refers to the enzymatic pathway to produce isobutanol. From time to time “isobutanol biosynthetic pathway” is used synonymously with “isobutanol production pathway”.

The term “2-butanone biosynthetic pathway” as used herein refers to an enzyme pathway to produce 2-butanone.

A “recombinant microbial host cell” is defined as a host cell that has been genetically manipulated to express a biosynthetic production pathway, wherein the host cell either produces a biosynthetic product in greater quantities relative to an unmodified host cell or produces a biosynthetic product that is not ordinarily produced by an unmodified host cell.

The term “fermentable carbon substrate” refers to a carbon source capable of being metabolized by the microorganisms such as those disclosed herein. Suitable fermentable carbon substrates include, but are not limited to, monosaccharides, such as glucose or fructose; disaccharides, such as lactose or sucrose; oligosaccharides; polysaccharides, such as starch, cellulose, or lignocellulose, hemicellulose; one-carbon substrates, amino acids, fatty acids; and a combination of these.

“Fermentation medium” as used herein means the mixture of water, sugars (fermentable carbon substrates), dissolved solids, microorganisms producing fermentation products, fermentation product and all other constituents of the material held in the fermentation vessel in which the fermentation product is being made by the reaction of fermentable carbon substrates to fermentation products, water and carbon dioxide (CO₂) by the microorganisms present. From time to time, as used herein the term “fermentation broth” and “fermentation mixture” can be used synonymously with “fermentation medium.”

The term “aerobic conditions” as used herein means growth conditions in the presence of oxygen.

The term “microaerobic conditions” as used herein means growth conditions with low levels of dissolved oxygen. For example, the oxygen level may be less than about 1% of air-saturation.

The term “anaerobic conditions” as used herein means growth conditions in the absence of oxygen.

“Butanol tolerance” or “tolerance to butanol” as used herein refers to the degree of effect butanol has on one or more of the following characteristics of a host cell in the presence of fermentation medium containing aqueous butanol: aerobic growth rate or anaerobic growth rate (typically a change in grams dry cell weight per liter fermentation medium per unit time, which may be expressed as “mu”), change in biomass (which may be expressed, for example, as a change in grams dry cell weight per liter fermentation medium, or as a change in optical density (O.D.)) over the course of a fermentation, volumetric productivity (which may be expressed in grams butanol produced per liter of fermentation medium per unit time), specific sugar consumption rate (“qS” typically expressed in grams sugar consumed per gram of dry cell weight of cells per hour), specific isobutanol production rate (“qP” typically expressed in grams butanol produced per gram of dry cell weight of cells per hour), or yield of butanol (grams of butanol produced per grams sugar consumed). It will be appreciated that increased butanol concentrations may impact one or more of the listed characteristics. Accordingly, an improvement in butanol tolerance can be demonstrated by a reduction or elimination of such impact on one or more of the listed characteristics.

The term “carbon substrate” refers to a carbon source capable of being metabolized by the recombinant host cells disclosed herein. Non-limiting examples of carbon substrates are provided herein and include, but are not limited to, monosaccharides, oligosaccharides, polysaccharides, ethanol, lactate, succinate, glycerol, carbon dioxide, methanol, glucose, fructose, sucrose, xylose, arabinose, dextrose, and mixtures thereof.

As used herein, the term “yield” refers to the amount of product per amount of carbon source in g/g. The yield may be exemplified for glucose as the carbon source. It is understood unless otherwise noted that yield is expressed as a percentage of the theoretical yield. In reference to a microorganism or metabolic pathway, “theoretical yield” is defined as the maximum amount of product that can be generated per total amount of substrate as dictated by the stoichiometry of the metabolic pathway used to make the product. For example, the theoretical yield for one typical conversion of glucose to isopropanol is 0.33 g/g. As such, a yield of isopropanol from glucose of 29.7 g/g would be expressed as 90% of theoretical or 90% theoretical yield. It is understood that while in the present disclosure the yield is exemplified for glucose as a carbon source, the invention can be applied to other carbon sources and the yield may vary depending on the carbon source used. One skilled in the art can calculate yields on various carbon sources.

The term “effective titer” as used herein, refers to the total amount of C3-C6 alcohol produced by fermentation per liter of fermentation medium. The total amount of C3-C6 alcohol includes: (i) the amount of C3-C6 alcohol in the fermentation medium; (ii) the amount of C3-C6 alcohol recovered from the organic extractant; and (iii) the amount of C3-C6 alcohol recovered from the gas phase, if gas stripping is used.

The term “effective rate” as used herein, refers to the total amount of C3-C6 alcohol produced by fermentation per liter of fermentation medium per hour of fermentation.

The term “effective yield” as used herein, refers to the amount of C3-C6 alcohol produced per unit of fermentable carbon substrate consumed by the biocatalyst.

The term “specific productivity” as used herein, refers to the g of C3-C6 alcohol produced per g of dry cell weight of cells per unit time.

As used herein the term “coding sequence” refers to a DNA sequence that encodes for a specific amino acid sequence. “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The terms “derivative” and “analog” refer to a polypeptide differing from the enzymes of the invention, but retaining essential properties thereof. The term “derivative” may also refer to a host cells differing from the host cells of the invention, but retaining essential properties thereof. Generally, derivatives and analogs are overall closely similar, and, in many regions, identical to the enzymes of the invention. The terms “derived-from”, “derivative” and “analog” when referring to enzymes of the invention include any polypeptides which retain at least some of the activity of the corresponding native polypeptide or the activity of its catalytic domain.

Derivatives of enzymes disclosed herein are polypeptides which may have been altered so as to exhibit features not found on the native polypeptide. Derivatives can be covalently modified by substitution (e.g. amino acid substitution), chemical, enzymatic, or other appropriate means with a moiety other than a naturally occurring amino acid (e.g., a detectable moiety such as an enzyme or radioisotope). Examples of derivatives include fusion proteins, or proteins which are based on a naturally occurring protein sequence, but which have been altered. For example, proteins can be designed by knowledge of a particular amino acid sequence, and/or a particular secondary, tertiary, and/or quaternary structure. Derivatives include proteins that are modified based on the knowledge of a previous sequence, natural or synthetic, which is then optionally modified, often, but not necessarily to confer some improved function. These sequences, or proteins, are then said to be derived from a particular protein or amino acid sequence. In some embodiments of the invention, a derivative must retain at least 50% identity, at least 60% identity, at least 70% identity, at least 80% identity, at least 85% identity, at least 87% identity, at least 90% identity, at least 91% identity, at least 92% identity, at least 93% identity, at least 94% identity, at least 95% identity, at least 96% identity, at least 97% identity, at least 98% identity, or at least 99% identity to the sequence the derivative is “derived-from.” In some embodiments of the invention, an enzyme is said to be derived-from an enzyme naturally found in a particular species if, using molecular genetic techniques, the DNA sequence for part or all of the enzyme is amplified and placed into a new host cell.

The term “fatty acids” refers to long-chain aliphatic acids (alkanoic acids) of varying chain lengths, from about C₁₂ to C₂₂ (although both longer and shorter chain-length acids are known). Generally, fatty acids are classified as saturated or unsaturated. The term “saturated fatty acids” refers to those fatty acids that have no carbon-carbon double bonds along their carbon backbone. In contrast, “unsaturated fatty acids” have carbon-carbon double bonds along their carbon backbones. “Monounsaturated fatty acids” have only one double bond along the carbon backbone, while “polyunsaturated fatty acids” (or “PUFAs”) have at least two double bonds along the carbon backbone. The structure of a fatty acid is represented by a simple notation system of “X:Y”, where X is the total number of carbon (C) atoms and Y is the number of double bonds. Table 1 lists non-limiting examples of various fatty acids and their nomenclature.

The term “cyclopropane fatty acid” as used herein, refers to fatty acids comprising one or more cyclopropane groups along their carbon backbone.

The term “C16 fatty acid” as used herein, refers to fatty acids comprising 16 carbons. The term “C18 fatty acid” as used herein, refers to fatty acids comprising 18 carbons.

The term “C18:1 fatty acid” as used herein, refers to fatty acids comprising 18 carbons and one carbon-carbon double bond. Non-limiting examples of C18:1 fatty acids are elaidic acid (C18:1 trans-9; IUPAC name: (E)-octadec-9-enoic acid) and trans-vaccenic acid (18:1 trans-11; IUPAC name: (E)-octadec-11-enoic acid).

The term “C18:2 fatty acid” as used herein, refers to fatty acids comprising 18 carbons and two carbon-carbon double bonds. Non-limiting examples of a C18:2 fatty acids are linoleic acid (C18:2 cis,cis-9,12; IUPAC name: (9Z,12Z)-octadeca-9,12-dienoic acid) and linolelaidic acid (C18:2 trans,trans-9,12; IUPAC name: (9E,12E)-octadeca-9,12-dienoic acid).

The term “C18:3 fatty acid” as used herein, refers to fatty acids comprising 18 carbons and three carbon-carbon double bonds. Non-limiting examples of C18:3 fatty acids are alpha-linolenic acid (C18:3 all cis-9,12,15; IUPAC name: (9Z,12Z,15Z)-octadeca-9,12,15-trienoic acid) and linolenelaidic acid (18:3 all trans-9,12,15; IUPAC name: (9E,12E,15E)-octadeca-9,12,15-trienoic acid).

As used herein, the term “COFA” refers to corn oil fatty acids (e.g., fatty acids from hydrolyzing corn oil).

The term “altered lipid profile” as used herein, refers to a yeast cell that comprises a different composition of fatty acids as compared to a wild-type cell grown under standard fermentation conditions. The composition of fatty acids in yeast cells can be determined by the methods known to those in the art, as well as the methods disclosed herein.

TABLE 1
Fatty acids and their nomenclature

| CX:Y | IUPAC name | Common name |
|---|---|---|
| C14:1, cis-9 | (Z)-tetradec-9-enoic acid | myristoleic acid |
| C14:1, trans-9 | (E)-tetradec-9-enoic acid | myristelaidic acid |
| C16:1, cis-9 | (Z)-hexadec-9-enoic acid | palmitoleic acid |
| C16:1, trans-9 | (E)-hexadec-9-enoic acid | palmitelaidic acid |
| C18:1, cis-6 | (Z)-octadec-6-enoic acid | petroselinic acid |
| C18:1, cis-9 | (Z)-octadec-9-enoic acid | oleic acid |
| C18:1, trans-9 | (E)-octadec-9-enoic acid | elaidic acid |
| C18:1, 9-ynoic | octadec-9-ynoic acid | stearolic acid |
| C18:1, cis-11 | (Z)-octadec-11-enoic acid | cis-vaccenic acid |
| C18:1, trans-11 | (E)-octadec-11-enoic acid | trans-vaccenic acid |
| C18:2, cis-9,12 | (Z)-octadeca-9,12-dienoic acid | linoleic acid |
| C18:2, trans-9,12 | (9E,12E)-octadeca-9,12-dienoic acid | linolenelaidic acid |
| C18:3, cis-6,9,12 | (6Z,9Z,12Z)-octadeca-6,9,12-trienoic acid | γ-linolenic acid |
| C18:3, cis-9,12,15 | (9Z,12Z,15Z)-octadeca-9,12,15-trienoic acid | linolenic acid |
| C18:3, trans- | (6E,9E,12E)-octadeca-6,9,12-trienoic acid | γ-linolenic acid |
| 9,12,15 | | |
| C20:1, cis-11 | (Z)-icos-11-enoic acid | gondoic acid |
| C20:4, cis-5,8,11,14 | (5Z,8Z,11Z,14Z)-icos-5,8,11,14-tetraenoic acid | arachidonic acid |
| C22:1, cis-13 | (Z)-docos-13-enoic acid | erucic acid |
| C22:1, trans-13 | (E)-docos-13-enoic acid | brassidic acid |
| C24:1, cis-15 | (Z)-tetracos-15-enoic acid | nervonic acid |

### Altering the Lipid Profile

The microorganisms of the present invention comprise an altered lipid profile. Specifically, the altered lipid profile results from an increase in (1) the concentration of C18:1, C18:2, and/or C18:3 fatty acids, (2) the ratio of unsaturated fatty acids to saturated fatty acids, (3) the ratio of C18 to C16 fatty acids, and/or (4) the concentration of cyclopropane fatty acids as compared to a yeast cell without the altered lipid profile.

One method to increase the concentration of C18:1, C18:2, and C18:3 fatty acids and/or the ratio of unsaturated to saturated fatty acids in the cell membrane is to engineer the microorganism to heterologously express a gene encoding a fatty acid desaturase enzyme. The term “fatty acid desaturase” refers to an enzyme that catalyzes the removal of two hydrogen atoms from a fatty acid, resulting in a carbon/carbon double bond. “Delta” or “Δ” fatty acid desaturases create the double bond at a fixed position from the carboxyl group of a fatty acid. Delta-9 desaturases are known by the EC number 1.14.19.1. These enzymes create a double bond at the ninth position from the carboxyl group of a fatty acid. Likewise, delta-12 desaturases create a double bond at the 12th position from the carboxyl group of a fatty acid. Delta-12 desaturases are known by the EC number 1.14.19.6. In some embodiments a microorganism is engineered to express a gene encoding a fatty acid desaturase enzyme. In some embodiments the fatty acid desaturase is selected from (a) a fatty acid desaturase having the EC number 1.14.19.1; (b) a fatty acid desaturase having the EC number 1.14.19.6; (c) a polypeptide that has at least 90% identity to any one or more of SEQ ID NOs: 1, 9, or 2; (d) a polypeptide encoded by a nucleic acid sequence that has at least 90% identity to any one or more of SEQ ID NOs: 3, 10, or 4; (e) a polypeptide encoded by a nucleic acid sequence that is complementary to any one or more of SEQ ID NOs: 3, 10, or 4; (f) a polypeptide encoded by a nucleic acid sequence that hybridizes under stringent conditions any one or more of SEQ ID NOs: 3, 10, or 4; or (g) any two or more of (a), (b), (c), (d), (e), or (f). It may be desirable to codon-optimize a heterologous coding region for expression in a yeast cell. Methods for codon-optimization are well known in the art.

One method to increase the concentration of cyclopropane fatty acids in the cell membrane is to engineer the microorganism to heterologously express a gene encoding a cyclopropane fatty acid synthase enzyme. Cyclopropane fatty acid synthases are known by the EC number 2.1.1.79. In some embodiments a microorganism is engineered to express a gene encoding a cyclopropane fatty acid synthase enzyme. In some embodiments the fatty acid desaturase is selected from (a) a cyclopropane fatty acid synthase having the EC number 2.1.1.79; (b) a polypeptide that has at least 90% identity to any one or more of SEQ ID NOs: 5 or 6; (c) a polypeptide encoded by a nucleic acid sequence that has at least 90% identity to any one or more of SEQ ID NOs: 7 or 8; (d) a polypeptide encoded by a nucleic acid sequence that is complementary to any one or more of SEQ ID NOs: 7 or 8; (e) a polypeptide encoded by a nucleic acid sequence that hybridizes under stringent conditions any one or more of SEQ ID NOs: 7 or 8; (f) any two or more of (a), (b), (c), (d), or (e). It may be desirable to codon-optimize a heterologous coding region for expression in a yeast cell. Methods for codon-optimization are well known in the art. Other methods for increasing the concentration of cyclopropane fatty acids are described in U.S. Pat. No. 8,518,678, herein incorporated by reference.

In the microorganisms of the present invention, the substrate for cyclopropane fatty acid synthase is present in the cell such that the expression of cyclopropane fatty acid synthase leads to increased concentration of cyclopropane fatty acid in the cell. The substrate, which is a cis unsaturated moiety in a fatty acid of a membrane phospholipid, is either endogenous to the cell or is derived from unsaturated fatty acids provided exogenously to the cell. The fatty acid substrates that may be present in the cell or provided to the cell, such as in the growth medium, include but are not limited to oleic acid (C18:1 cis-9), cis-vaccenic acid (C18:1 cis-11) and palmitoleic acid (C16:1). Cyclopropane fatty acid synthase enzymes may prefer different substrates and produce different cyclopropane fatty acids. For example, the cyclopropane fatty acid synthase encoded enzyme of L. plantarum (SEQ ID NO: 5) converts the endogenous substrate cis-vaccenic acid to the cyclopropane fatty acid lactobacillic acid (cis-11,12 methylene-octadecanoic acid). The cfa encoded enzyme of E. coli (SEQ ID NO: 43) converts endogenous cis-vaccenic acid (C18:1 cis-11) and palmitoleic acid (C16:1 cis-9) substrates to the corresponding 19cyclo and 17cyclopropane fatty acids. The L. plantarum cfa2 encoded enzyme (SEQ ID NO: 6) converts oleic acid to the cyclopropane fatty acid dihydrosterculic acid when this substrate is fed to the cells in the growth medium. One skilled in the art can readily without undue experimentation determine a substrate for a particular cyclopropane fatty acid synthase and assess that it is present in the cell, or if not, provide it in the growth medium.

It may also be desirable to increase the ratio of C18 to C16 fatty acids in the cell membrane. One method to increase the ratio of C18 to C16 fatty acids is to engineer the microorganism to heterologously express a gene encoding a fatty acid elongase. The term “fatty acid elongase” refers to a polypeptide component of a multienzyme complex that can elongate a fatty acid carbon chain to produce a mono- or polyunsaturated fatty acid that is 2 carbons longer than the fatty acid substrate that the elongase acts upon. This process of elongation occurs in a multi-step mechanism in association with fatty acid synthase, whereby CoA is the acyl carrier. (Lassner et al., The Plant Cell (1996) 8:281-292). Briefly, malonyl-CoA is condensed with a long-chain acyl-CoA to yield CO₂ and a β-ketoacyl-CoA (where the acyl moiety has been elongated by two carbon atoms). Subsequent reactions include reduction to β-hydroxyacyl-CoA, dehydration to an enoyl-CoA and a second reduction to yield the elongated acyl-CoA. Examples of reactions catalyzed by elongases are the conversion of γ-linoleic acid to dihomo-γ-linoleic acid, stearidonic acid to eicosa-tetraenoic acid, and eicosa-pentaenoic acid to docosa-pentaenoic acid. Accordingly, elongases can have different specificities (e.g., a C16/18 or C16 elongase will prefer a C16 substrate, a C18/20 or C18 elongase will prefer a C18 substrate, and a C20/22 or C20 elongase will prefer a C20 substrate). In some embodiments that fatty acid elongase is selected from (a) a polypeptide that has at least 90% identity to any one or more of SEQ ID NOs: 15, 16, or 11; (b) a polypeptide encoded by a nucleic acid sequence that has at least 90% identity to any one or more of SEQ ID NOs: 17, 18, 12; (c) a polypeptide encoded by a nucleic acid sequence that is complementary to any one or more of SEQ ID NOs: 17, 18, 12; (d) a polypeptide encoded by a nucleic acid sequence that hybridizes under stringent conditions any one or more of SEQ ID NOs: 17, 18, 12; or (e) any two or more of (a), (b), (c), or (d). It may be desirable to codon-optimize a heterologous coding region for optimal expression in a yeast cell. Methods for codon-optimization are well known in the art.

In some embodiments that ratio of C18:1 to C16:1 fatty acids is increased by at least about 10%, at least about 20%, at least about 30%, at least about 40%, and at least about 50% when the microorganism is engineered to express a 49 fatty acid desaturase. In some embodiments the concentration of C18:1 fatty acids comprises at least about 30%, at least about 40%, at least about 50%, at least about 60%, at least about 70%, at least about 75% of the total fatty acid content when the microorganism is engineered to express a 49 fatty acid desaturase alone or along with expression of a C16 elongase. In some embodiments the concentration of C18:2 fatty acids comprises at least about 20%, at least about 30%, at least about 40%, at least about 45% of the total fatty acid content when the microorganism is engineered to express a Δ12 fatty acid desaturase alone or along with expression of a Δ9 desaturase or a Δ9 desaturase and a C16 elongase.

In some embodiments the concentration of C18 fatty acids comprises at least about 40%, at least about 50%, at least about 60%, at least about 70%, at least about 80% of the total fatty acid content when the microorganism is engineered to express a fatty acid elongase. In some embodiments microorganisms engineered to express a fatty acid elongase have at least about a 1.1-fold to at least about a 20-fold increase in the production of isobutanol when cultured in the presence of isobutanol. In some embodiments microorganisms engineered to express a fatty acid elongase have at least about a 1.1-fold, at least about a 1.2-fold, at least about a 1.3-fold, at least about a 1.4-fold, at least about a 1.5-fold increase in cell density when cultured in the presence of isobutanol.

In some embodiments the concentration of cyclopropane fatty acids comprises at least about 1%, at least about 2%, at least about 3%, at least about 4%, at least about 5% of the total fatty acid content when the microorganism is engineered to express a cyclopropane fatty acid synthase.

In some embodiments the concentration of C18:1 fatty acids comprises at least about 30%, at least about 40%, at least about 50%, at least about 60%, at least about 70%, at least about 80% when the microorganism is engineered to express a 49 fatty acid desaturase and a fatty acid elongase. In some embodiments the ratio of C18 to C16 fatty acids is increased by at least about 2-fold, at least about 3-fold, at least about 4-fold when the microorganism is engineered to express a 49 fatty acid desaturase and a fatty acid elongase.

The sequences of the fatty acid desaturase, cyclopropane fatty acid synthase, and fatty acid elongase gene coding regions provided herein may be used to identify other homologs in nature. For example each of the fatty acid desaturase, cyclopropane fatty acid synthase, and fatty acid elongase gene nucleic acid fragments described herein may be used to isolate genes encoding homologous proteins. Isolation of homologous genes using sequence-dependent protocols is well known in the art. Examples of sequence-dependent protocols include, but are not limited to: 1) methods of nucleic acid hybridization; 2) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor, S. et al., Proc. Natl. Acad. Sci. U.S.A. 82:1074 (1985); or strand displacement amplification (SDA), Walker, et al., Proc. Natl. Acad. Sci. U.S.A., 89:392 (1992)]; and 3) methods of library construction and screening by complementation.

For example, genes encoding similar proteins or polypeptides to the fatty acid desaturase, cyclopropane fatty acid synthase, and fatty acid elongase genes provided herein could be isolated directly by using all or a portion of the instant nucleic acid fragments as DNA hybridization probes to screen libraries from any desired organism using methodology well known to those skilled in the art. Specific oligonucleotide probes based upon the disclosed nucleic acid sequences can be designed and synthesized by methods known in the art (Maniatis, supra). Moreover, the entire sequences can be used directly to synthesize DNA probes by methods known to the skilled artisan (e.g., random primers DNA labeling, nick translation or end-labeling techniques), or RNA probes using available in vitro transcription systems. In addition, specific primers can be designed and used to amplify a part of (or full-length of) the instant sequences. The resulting amplification products can be labeled directly during amplification reactions or labeled after amplification reactions, and used as probes to isolate full-length DNA fragments by hybridization under conditions of appropriate stringency. Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotides as specific hybridization probes in the Diagnosis of Genetic Disorders”, in Human Genetic Diseases: A Practical Approach, K. E. Davis Ed., (1986) pp. 33-50, IRL: Herndon, Va.; and Rychlik, W., In Methods in Molecular Biology, White, B. A. Ed., (1993) Vol. 15, pp 31-39, PCR Protocols: Current Methods and Applications. Humania: Totowa, N.J.).

Generally two short segments of the described sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the described nucleic acid fragments, and the sequence of the other primer takes advantage of the presence of the polyadenylic acid tracts to the 3′ end of the mRNA precursor encoding microbial genes.

Alternatively, the second primer sequence may be based upon sequences derived from the cloning vector. For example, the skilled artisan can follow the RACE protocol (Frohman et al., Proc. Natl. Acad. Sci. U.S.A. 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (e.g., BRL, Gaithersburg, Md.), specific 3′ or 5′ cDNA fragments can be isolated (Ohara et al., Proc. Natl. Acad. Sci. USA 86:5673 (1989); Loh et al., Science 243:217 (1989)).

Alternatively, the provided fatty acid desaturase, cyclopropane fatty acid synthase, and fatty acid elongase gene encoding sequences may be employed as hybridization reagents for the identification of homologs. The basic components of a nucleic acid hybridization test include a probe, a sample suspected of containing the gene or gene fragment of interest, and a specific hybridization method. Probes are typically single-stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions that will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally, a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kdal), polyvinylpyrrolidone (about 250-500 kdal) and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents that include a variety of polar water-soluble or swellable agents (e.g., polyethylene glycol), anionic polymers (e.g., polyacrylate or polymethylacrylate) and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

Another method to increase the concentration of C18:1, C18:2, and C18:3 fatty acids, the ratio of unsaturated to saturated fatty acids, the concentration of cyclopropane fatty acids, and/or the ratio of C18 to C16 fatty acids is to contact the cells with C18:1, C18:2, C18:3, cyclopropane fatty acids, and/or COFA. Methods for contacting cells with fatty acids are further described in U.S. Patent Appl. Pub. No. 2011/0312053, U.S. Patent Appl. Pub. No. 2011/0195505, and U.S. Patent Appl. Pub. No. 2010/0136641, all herein incorporated by reference.

### Increased Tolerance to Butanol

A microorganism of the present invention has improved tolerance to butanol. The tolerance of microorganisms with an altered lipid profile may be assessed by assaying their growth in concentrations of butanol that are detrimental to growth of a strain not comprising an altered lipid profile. Improved tolerance is to butanol compounds such as 1-butanol, 2-butanol, or isobutanol, or a combination thereof. The amount of tolerance improvement will vary depending on the inhibiting chemical and its concentration, growth conditions and the specific genetically modified strain. For example, as shown in Example 4 herein, strains comprising an increased concentration of C18:1 fatty acids reached a higher OD and produced more isobutanol than a strain not comprising an altered lipid profile.

Tolerance to butanol can also be shown by an increase in aerobic growth rate or anaerobic growth rate, in biomass over the course of a fermentation, in volumetric productivity, in specific sugar consumption rate, in specific isobutanol production rate, or in the yield of butanol. It will be appreciated that increased butanol concentrations may impact one or more of the listed characteristics. Accordingly, an improvement in butanol tolerance can be demonstrated by a reduction or elimination of such impact on one or more of the listed characteristics.

Yeast strains can be modified to comprise an altered lipid profile. In some embodiments the microorganism is modified to express one or more of a fatty acid desaturase, a cyclopropane fatty acid synthase, and a fatty acid elongase. The resultant strains can then be transformed to comprise an engineered isobutanol biosynthetic pathway. The resultant engineered isobutanol biosynthetic pathway comprising strains obtained from the transformations can then be monitored over time to measure their rate of butanol tolerance. In accordance with the present invention, yeast strains modified to comprise an altered lipid profile have an increased growth rate or final cell density in the culture, and may produce more isobutanol compared to a strain that does not comprise an altered lipid profile. (See Tables 16 and 17). In some embodiments a microorganism engineered to express an engineered isobutanol biosynthetic pathway is fed fatty acids to alter its lipid profile.

Those skilled in the art will know that the microorganisms of the present invention can be modified to comprise other modifications known to confer tolerance to butanol.

### Pyruvate Decarboxylase

The term “pyruvate decarboxylase” refers to an enzyme that catalyzes the decarboxylation of pyruvic acid to acetaldehyde and carbon dioxide. Pyruvate decarboxylases are known by the EC number 4.1.1.1. These enzymes are found in a number of yeast, including Saccharomyces cerevisiae (GenBank No: NP_013145 (SEQ ID NO: 44), CAA97705 (SEQ ID NO: 45), CAA97091 (SEQ ID NO: 46)).

U.S. Appl. Pub. No. 2009/0305363 (incorporated by reference) discloses increased conversion of pyruvate to acetolactate by engineering yeast for expression of a cytosol-localized acetolactate synthase and substantial elimination of pyruvate decarboxylase activity. A genetic modification which has the effect of reducing glucose repression wherein the yeast production host cell is pdc- is described in U.S. Appl. Publication No. 2011/0124060, incorporated herein by reference. In some embodiments, the pyruvate decarboxylase that is deleted or down regulated is selected from the group consisting of: PDC1, PDC5, PDC6, and combinations thereof. In some embodiments, the pyruvate decarboxylase is selected from those enzymes in Table 2.

TABLE 2
SEQ ID Numbers of PDC Target Gene Coding Regions and Proteins.

| | SEQ ID | SEQ |
|---|---|---|
| Description | NO: Nucleic Acid | ID NO: Amino Acid |
| PDC1 pyruvate decarboxylase | 47 | 44 |
| from Saccharomyces cerevisiae | | |
| PDC5 pyruvate decarboxylase | 48 | 45 |
| from Saccharomyces cerevisiae | | |
| PDC6 pyruvate decarboxylase | 49 | 46 |
| Saccharomyces cerevisiae | | |
| pyruvate decarboxylase from | 50 | 51 |
| Candida glabrata | | |
| PDC1 pyruvate decarboxylase | 52 | 53 |
| from Pichia stipites | | |
| PDC2 pyruvate decarboxylase | 54 | 55 |
| from Pichia stipites | | |
| pyruvate decarboxylase from | 56 | 57 |
| Kluyveromyces lactis | | |
| pyruvate decarboxylase from | 58 | 59 |
| Yarrowia lipolytica | | |
| pyruvate decarboxylase from | 60 | 61 |
| Schizosaccharomyces pombe | | |
| pyruvate decarboxylase from | 62 | 63 |
| Zygosaccharomyces rouxii | | |

Yeasts may have one or more genes encoding pyruvate decarboxylase. For example, there is one gene encoding pyruvate decarboxylase in Candida glabrata and Schizosaccharomyces pombe, while there are three isozymes of pyruvate decarboxylase encoded by the PDC1, PCD5, and PDC6 genes in Saccharomyces. In some embodiments, in the present yeast cells at least one PDC gene is inactivated. If the yeast cell used has more than one expressed (active) PDC gene, then each of the active PDC genes may be modified or inactivated thereby producing a pdc-cell. For example, in S. cerevisiae the PDC1, PDC5, and PDC6 genes may be modified or inactivated. If a PDC gene is not active under the fermentation conditions to be used then such a gene would not need to be modified or inactivated.

Other target genes, such as those encoding pyruvate decarboxylase proteins having at least 70-75%, at least 75-80%, at least 80-85%, at least 85%-90%, at least 90%-95%, or at least 96%, at least 97%, at least 98%, or at least 99% sequence identity to the pyruvate decarboxylases of SEQ ID NOs: 44, 45, 46, 51, 53, 55, 57, 59, 61, or 63 may be identified in the literature and in bioinformatics databases well known to the skilled person. In addition, the methods described herein for identifying fatty acid desaturase, cyclopropane fatty acid synthase, or fatty acid elongase gene homologs can be employed to identify pyruvate decarboxylase genes in microorganisms of interest using the pyruvate decarboxylase sequences provided herein.

### Polypeptides and Polynucleotides for Use in the Invention

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. A polypeptide may be derived from a natural biological source or produced by recombinant technology, but is not necessarily translated from a designated nucleic acid sequence. It may be generated in any manner, including by chemical synthesis. The polypeptides used in this invention comprise full-length polypeptides and fragments thereof.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purposes of the invention, as are native or recombinant polypeptides which have been separated, fractionated, or partially or substantially purified by any suitable technique.

A polypeptide of the invention may be of a size of about 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

Also included as polypeptides of the present invention are derivatives, analogs, or variants of the foregoing polypeptides, and any combination thereof. The terms “active variant,” “active fragment,” “active derivative,” and “analog” refer to polypeptides of the present invention. Variants of polypeptides of the present invention include polypeptides with altered amino acid sequences due to amino acid substitutions, deletions, and/or insertions. Variants may occur naturally or be non-naturally occurring. Non-naturally occurring variants may be produced using art-known mutagenesis techniques. Variant polypeptides may comprise conservative or non-conservative amino acid substitutions, deletions and/or additions. Derivatives of polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of a polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

A “fragment” is a unique portion of a polypeptide or other enzyme used in the invention which is identical in sequence to but shorter in length than the parent full-length sequence. A fragment may comprise up to the entire length of the defined sequence, minus one amino acid residue. For example, a fragment may comprise from 5 to 1000 contiguous amino acid residues. A fragment may be at least 5, 10, 15, 20, 25, 30, 40, 50, 60, 75, 100, 150, 250 or at least 500 contiguous amino acid residues in length. Fragments may be preferentially selected from certain regions of a molecule. For example, a polypeptide fragment may comprise a certain length of contiguous amino acids selected from the first 100 or 200 amino acids of a polypeptide as shown in a certain defined sequence. Clearly these lengths are exemplary, and any length that is supported by the specification, including the Sequence Listing, tables, and figures, may be encompassed by the present embodiments.

Alternatively, recombinant variants encoding these same or similar polypeptides can be synthesized or selected by making use of the “redundancy” in the genetic code. Various codon substitutions, such as the silent changes which produce various restriction sites, may be introduced to optimize cloning into a plasmid or viral vector or expression in a host cell system.

Preferably, amino acid “substitutions” are the result of replacing one amino acid with another amino acid having similar structural and/or chemical properties, i.e., conservative amino acid replacements, or they can be result of replacing one amino acid with an amino acid having different structural and/or chemical properties, i.e., non-conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions can be made by selecting the differences in polarity, charge, solubility, hydrophobicity, hydrophilicity, or the amphipathic nature of any of these amino acids. “Insertions” or “deletions” are preferably in the range of about 1 to about 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

By a polypeptide having an amino acid or polypeptide sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the references sequence.

As a practical matter, whether any particular polypeptide is at least 60%, 65%, 70%, 75%, 80%, 85%, 90%, 95%, 96%, 97%, 98%, or 99% identical to a reference polypeptide can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment, the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty-0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case, the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention.

Polypeptides and other enzymes suitable for use in the present invention and fragments thereof are encoded by polynucleotides. The term “polynucleotide” is intended to encompass a singular nucleic acid as well as plural nucleic acids, and refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. Polynucleotides according to the present invention further include such molecules produced synthetically. Polynucleotides of the invention may be native to the host cell or heterologous. In addition, a polynucleotide or a nucleic acid may be or may include a regulatory element such as a promoter, ribosome binding site, or a transcription terminator.

In certain embodiments, the polynucleotide or nucleic acid is DNA. In the case of DNA, a polynucleotide comprising a nucleic acid, which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. Other transcription control elements, besides a promoter, for example enhancers, operators, repressors, and transcription termination signals, can be operably associated with the polynucleotide. Suitable promoters and other transcription control regions are disclosed herein.

A polynucleotide or polypeptide sequence can be referred to as “isolated,” in which it has been placed in an environment other than its native environment or is produced synthetically or is a non-naturally occurring, or engineered, sequence. For example, a heterologous polynucleotide encoding a polypeptide or polypeptide fragment having enzymatic activity (e.g., the ability to convert a substrate to xylulose) contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide fragment in the form of a polymer of DNA can be comprised of one or more segments of cDNA, genomic DNA, or synthetic DNA.

The term “gene” refers to a nucleic acid fragment that is capable of being expressed as a specific protein, optionally including regulatory sequences preceding (5′ non-coding sequences) and following (3′ non-coding sequences) the coding sequence.

As used herein, a “coding region” or “ORF” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ non-translated regions, and the like, are not part of a coding region.

A variety of translation control elements are known to those of ordinary skill in the art. These include, but are not limited to ribosome binding sites, translation initiation and termination codons, and elements derived from viral systems (particularly an internal ribosome entry site, or IRES). In other embodiments, a polynucleotide of the present invention is RNA, for example, in the form of messenger RNA (mRNA). RNA of the present invention may be single stranded or double stranded.

Polynucleotide and nucleic acid coding regions of the present invention may be associated with additional coding regions which encode secretory or signal peptides, which direct the secretion of a polypeptide encoded by a polynucleotide of the present invention.

As used herein, the term “transformation” refers to the transfer of a nucleic acid fragment into the genome of a host organism, resulting in genetically stable inheritance. Host organisms containing the transformed nucleic acid fragments are referred to as “recombinant” or “transformed” organisms.

The term “expression,” as used herein, refers to the transcription and stable accumulation of sense (mRNA) or antisense RNA derived from the nucleic acid fragment of the invention. Expression may also refer to translation of mRNA into a polypeptide.

The terms “plasmid,” “vector,” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “artificial” refers to a synthetic, or non-host cell derived composition, e.g., a chemically-synthesized oligonucleotide.

As used herein, “native” refers to the form of a polynucleotide, gene, or polypeptide as found in nature with its own regulatory sequences, if present.

The term “endogenous,” when used in reference to a polynucleotide, a gene, or a polypeptide refers to a native polynucleotide or gene in its natural location in the genome of an organism, or for a native polypeptide, is transcribed and translated from this location in the genome.

The term “heterologous” when used in reference to a polynucleotide, a gene, or a polypeptide refers to a polynucleotide, gene, or polypeptide not normally found in the host organism. “Heterologous” also includes a native coding region, or portion thereof, that is reintroduced into the source organism in a form that is different from the corresponding native gene, e.g., not in its natural location in the organism's genome. The heterologous polynucleotide or gene may be introduced into the host organism by, e.g., gene transfer. A heterologous gene may include a native coding region with non-native regulatory regions that is reintroduced into the native host. A “transgene” is a gene that has been introduced into the genome by a transformation procedure.

“Deletion” or “deleted” or “disruption” or “disrupted” or “elimination” or “eliminated” used with regard to a gene or set of genes describes various activities for example, 1) deleting coding regions and/or regulatory (promoter) regions, 2) inserting exogenous nucleic acid sequences into coding regions and/regulatory (promoter) regions, and 3) altering coding regions and/or regulatory (promoter) regions (for example, by making DNA base pair changes). Such changes would either prevent expression of the protein of interest or result in the expression of a protein that is non-functional/shows no activity. Specific disruptions may be obtained by random mutation followed by screening or selection, or, in cases where the gene sequences are known, specific disruptions may be obtained by direct intervention using molecular biology methods know to those skilled in the art.

The terms “mutation” or “genetic modification” as used herein indicate any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring or spontaneous. In other embodiments, the mutations are the result of treatment with mutagenic agents such as ethyl methanesulfonate or ultraviolet light. In still other embodiments, the mutations in the microorganism genome are the result of genetic engineering.

The term “recombinant genetic expression element” refers to a nucleic acid fragment that expresses one or more specific proteins, including regulatory sequences preceding (5′ non-coding sequences) and following (3′ termination sequences) coding sequences for the proteins. A chimeric gene is a recombinant genetic expression element. The coding regions of an operon may form a recombinant genetic expression element, along with an operably linked promoter and termination region.

“Regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, operators, repressors, transcription termination signals, translation leader sequences, introns, polyadenylation recognition sequences, RNA processing site, effector binding site and stem-loop structure.

The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. “Inducible promoters,” on the other hand, cause a gene to be expressed when the promoter is induced or turned on by a promoter-specific signal or molecule. It is further recognized that since in most cases the exact boundaries of regulatory sequences have not been completely defined, DNA fragments of different lengths may have identical promoter activity. For example, it will be understood that “FBA1 promoter” can be used to refer to a fragment derived from the promoter region of the FBA1 gene.

The term “terminator” as used herein refers to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence. It is recognized that since in most cases the exact boundaries of regulatory sequences have not been completely defined, DNA fragments of different lengths may have identical terminator activity. For example, it will be understood that “CYC1 terminator” can be used to refer to a fragment derived from the terminator region of the CYC1 gene.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 3. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 3
The Standard Genetic Code

| T | C | A | G | |
|---|---|---|---|---|
| T | TTT Phe (F) | TCT Ser (S) | TAT Tyr (Y) | TGT Cys (C) |
| | TTC ″ | TCC ″ | TAC ″ | TGC |
| | TTA Leu (L) | TCA ″ | TAA Ter | TGA Ter |
| | TTG ″ | TCG ″ | TAG Ter | TGG Trp (W) |
| C | CTT Leu (L) | CCT Pro (P) | CAT His (H) | CGT Arg (R) |
| | CTC ″ | CCC ″ | CAC ″ | CGC ″ |
| | CTA ″ | CCA ″ | CAA Gln (Q) | CGA ″ |
| | CTG ″ | CCG ″ | CAG ″ | CGG ″ |
| A | ATT Ile (I) | ACT Thr (T) | AAT Asn (N) | AGT Ser (S) |
| | ATC ″ | ACC ″ | AAC ″ | AGC ″ |
| | ATA ″ | ACA ″ | AAA Lys (K) | AGA Arg (R) |
| | ATG Met | ACG ″ | AAG ″ | AGG ″ |
| | (M) | | | |
| G | GTT Val (V) | GCT Ala (A) | GAT Asp (D) | GGT Gly (G) |
| | GTC ″ | GCC ″ | GAC ″ | GGC ″ |
| | GTA ″ | GCA ″ | GAA Glu (E) | GGA ″ |
| | GTG ″ | GCG ″ | GAG ″ | GGG ″ |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon-optimization.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited Jun. 26, 2012), and these tables can be adapted in a number of ways. See Nakamura, Y., et al. Nucl. Acids Res. 28:292 (2000). Codon usage tables for yeast, calculated from GenBank Release 128.0 [15 Feb. 2002], are reproduced below as Table 4. This table uses mRNA nomenclature, and so instead of thymine (T) which is found in DNA, the tables use uracil (U) which is found in RNA. The Table has been adapted so that frequencies are calculated for each amino acid, rather than for all 64 codons.

TABLE 4
Codon Usage Table for Saccharomyces cerevisiae Genes

| | | | Frequency per |
|---|---|---|---|
| Amino Acid | Codon | Number | thousand |
| Phe | UUU | 170666 | 26.1 |
| Phe | UUC | 120510 | 18.4 |
| Leu | UUA | 170884 | 26.2 |
| Leu | UUG | 177573 | 27.2 |
| Leu | CUU | 80076 | 12.3 |
| Leu | CUC | 35545 | 5.4 |
| Leu | CUA | 87619 | 13.4 |
| Leu | CUG | 68494 | 10.5 |
| Ile | AUU | 196893 | 30.1 |
| Ile | AUC | 112176 | 17.2 |
| Ile | AUA | 116254 | 17.8 |
| Met | AUG | 136805 | 20.9 |
| Val | GUU | 144243 | 22.1 |
| Val | GUC | 76947 | 11.8 |
| Val | GUA | 76927 | 11.8 |
| Val | GUG | 70337 | 10.8 |
| Ser | UCU | 153557 | 23.5 |
| Ser | UCC | 92923 | 14.2 |
| Ser | UCA | 122028 | 18.7 |
| Ser | UCG | 55951 | 8.6 |
| Ser | AGU | 92466 | 14.2 |
| Ser | AGC | 63726 | 9.8 |
| Pro | CCU | 88263 | 13.5 |
| Pro | CCC | 44309 | 6.8 |
| Pro | CCA | 119641 | 18.3 |
| Pro | CCG | 34597 | 5.3 |
| Thr | ACU | 132522 | 20.3 |
| Thr | ACC | 83207 | 12.7 |
| Thr | ACA | 116084 | 17.8 |
| Thr | ACG | 52045 | 8.0 |
| Ala | GCU | 138358 | 21.2 |
| Ala | GCC | 82357 | 12.6 |
| Ala | GCA | 105910 | 16.2 |
| Ala | GCG | 40358 | 6.2 |
| Tyr | UAU | 122728 | 18.8 |
| Tyr | UAC | 96596 | 14.8 |
| His | CAU | 89007 | 13.6 |
| His | CAC | 50785 | 7.8 |
| Gln | CAA | 178251 | 27.3 |
| Gln | CAG | 79121 | 12.1 |
| Asn | AAU | 233124 | 35.7 |
| Asn | AAC | 162199 | 24.8 |
| Lys | AAA | 273618 | 41.9 |
| Lys | AAG | 201361 | 30.8 |
| Asp | GAU | 245641 | 37.6 |
| Asp | GAC | 132048 | 20.2 |
| Glu | GAA | 297944 | 45.6 |
| Glu | GAG | 125717 | 19.2 |
| Cys | UGU | 52903 | 8.1 |
| Cys | UGC | 31095 | 4.8 |
| Trp | UGG | 67789 | 10.4 |
| Arg | CGU | 41791 | 6.4 |
| Arg | CGC | 16993 | 2.6 |
| Arg | CGA | 19562 | 3.0 |
| Arg | CGG | 11351 | 1.7 |
| Arg | AGA | 139081 | 21.3 |
| Arg | AGG | 60289 | 9.2 |
| Gly | GGU | 156109 | 23.9 |
| Gly | GGC | 63903 | 9.8 |
| Gly | GGA | 71216 | 10.9 |
| Gly | GGG | 39359 | 6.0 |
| Stop | UAA | 6913 | 1.1 |
| Stop | UAG | 3312 | 0.5 |
| Stop | UGA | 4447 | 0.7 |

By utilizing this or similar tables, one of ordinary skill in the art can apply the frequencies to any given polypeptide sequence, and produce a nucleic acid fragment of a codon-optimized coding region which encodes the polypeptide, but which uses codons optimal for a given species.

Randomly assigning codons at an optimized frequency to encode a given polypeptide sequence can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the VectorNTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences, e.g., the “JAVA Codon Adaptation Tool” at http://www.jcat.de/ (visited Jun. 25, 2012) and the “Codon optimization tool” available at http://www.entelechon.com/2008/10/backtranslation-tool/ (visited Jun. 25, 2012). Constructing a rudimentary algorithm to assign codons based on a given frequency can also easily be accomplished with basic mathematical functions by one of ordinary skill in the art.

Standard recombinant DNA and molecular cloning techniques used here are well known in the art and are described by Sambrook et al. (Sambrook, Fritsch, and Maniatis, Molecular Cloning: A Laboratory Manual, Second Edition, Cold Spring Harbor Laboratory Press, Cold Spring Harbor, N.Y., 1989) (hereinafter “Maniatis”); and by Silhavy et al. (Silhavy et al., Experiments with Gene Fusions, Cold Spring Harbor Laboratory Press Cold Spring Harbor, N.Y., 1984); and by Ausubel, F. M. et al., (Ausubel et al., Current Protocols in Molecular Biology, published by Greene Publishing Assoc. and Wiley-Interscience, 1987).

### Biosynthetic Pathways

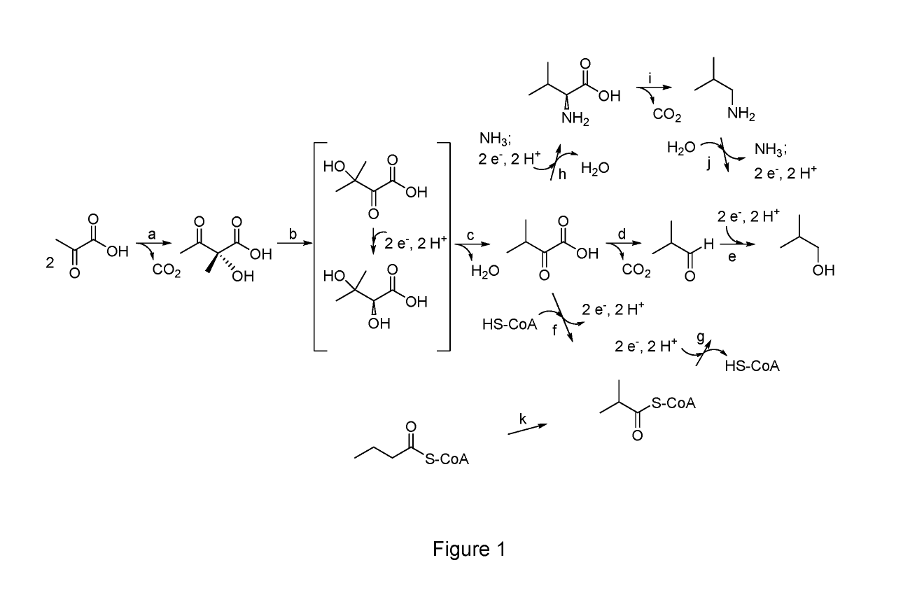

Biosynthetic pathways for the production of isobutanol that may be used include those described in U.S. Pat. Nos. 7,851,188 and 7,993,889, which are incorporated herein by reference. Isobutanol pathways are referred to with their lettering in FIG. 1. In one embodiment, the engineered isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
d) α-ketoisovalerate to isobutyraldehyde, which may be catalyzed, for example, by a branched-chain keto acid decarboxylase; and,
e) isobutyraldehyde to isobutanol, which may be catalyzed, for example, by a branched-chain alcohol dehydrogenase.

In another embodiment, the engineered isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by ketol-acid reductoisomerase;
c) 2,3-dihydroxyisovalerate to α-ketoisovalerate, which may be catalyzed, for example, by acetohydroxy acid dehydratase;
h) α-ketoisovalerate to valine, which may be catalyzed, for example, by transaminase or valine dehydrogenase;
i) valine to isobutylamine, which may be catalyzed, for example, by valine decarboxylase;
j) isobutylamine to isobutyraldehyde, which may be catalyzed by, for example, omega transaminase; and,
e) isobutyraldehyde to isobutanol, which may be catalyzed, for example, by a branched-chain alcohol dehydrogenase.

In another embodiment, the engineered isobutanol biosynthetic pathway comprises the following substrate to product conversions:
a) pyruvate to acetolactate, which may be catalyzed, for example, by acetolactate synthase;
b) acetolactate to 2,3-dihydroxyisovalerate, which may be catalyzed, for example, by acetohydroxy acid reductoisomerase;
c) 2,3-dihydroxyisovalerate to α-ketoisovalerate, which may be catalyzed, for example, by acetohydroxy acid dehydratase;
f) α-ketoisovalerate to isobutyryl-CoA, which may be catalyzed, for example, by branched-chain keto acid dehydrogenase;
g) isobutyryl-CoA to isobutyraldehyde, which may be catalyzed, for example, by acelylating aldehyde dehydrogenase; and,
e) isobutyraldehyde to isobutanol, which may be catalyzed, for example, by a branched-chain alcohol dehydrogenase.

In another embodiment, the isobutanol biosynthetic pathway comprises the substrate to product conversions shown as steps k, g, and e in FIG. 1.

Engineered biosynthetic pathways for the production of 1-butanol that may be used include those described in U.S. Patent Appl. Pub. No. 2008/0182308, which is incorporated herein by reference. In one embodiment, the 1-butanol biosynthetic pathway comprises the following substrate to product conversions:
a) acetyl-CoA to acetoacetyl-CoA, which may be catalyzed, for example, by acetyl-CoA acetyl transferase;
b) acetoacetyl-CoA to 3-hydroxybutyryl-CoA, which may be catalyzed, for example, by 3-hydroxybutyryl-CoA dehydrogenase;
c) 3-hydroxybutyryl-CoA to crotonyl-CoA, which may be catalyzed, for example, by crotonase;
d) crotonyl-CoA to butyryl-CoA, which may be catalyzed, for example, by butyryl-CoA dehydrogenase;
e) butyryl-CoA to butyraldehyde, which may be catalyzed, for example, by butyraldehyde dehydrogenase; and,
f) butyraldehyde to 1-butanol, which may be catalyzed, for example, by butanol dehydrogenase.

Engineered biosynthetic pathways for the production of 2-butanol that may be used include those described in U.S. Pat. No. 8,206,970 and U.S. Patent Appl. Pub. No. 2009/0155870, which are incorporated herein by reference. In one embodiment, the 2-butanol biosynthetic pathway comprises the following substrate to product conversions:
a) pyruvate to α-acetolactate, which may be catalyzed, for example, by acetolactate synthase;
b) α-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase;
e) 3-amino-2-butanol phosphate to 2-butanone, which may be catalyzed, for example, by aminobutanol phosphate phosphorylase; and,
f) 2-butanone to 2-butanol, which may be catalyzed, for example, by butanol dehydrogenase.

In another embodiment, the engineered 2-butanol biosynthetic pathway comprises the following substrate to product conversions:
a) pyruvate to α-acetolactate, which may be catalyzed, for example, by acetolactate synthase;
b) α-acetolactate to acetoin, which may be catalyzed, for example, by acetolactate decarboxylase;
c) acetoin to 2,3-butanediol, which may be catalyzed, for example, by butanediol dehydrogenase;
d) 2,3-butanediol to 2-butanone, which may be catalyzed, for example, by dial dehydratase; and,
e) 2-butanone to 2-butanol, which may be catalyzed, for example, by butanol dehydrogenase.

Engineered biosynthetic pathways for the production of 2-butanone that may be used include those described in U.S. Pat. No. 8,206,970 and U.S. Patent Appl. Pub. No. 2009/0155870, which are incorporated herein by reference. In one embodiment, the engineered 2-butanone biosynthetic pathway comprises the following substrate to product conversions:
a) pyruvate to α-acetolactate, which may be catalyzed, for example, by acetolactate synthase;
b) α-acetolactate to acetoin, which may be catalyzed, for example, by acetolactate decarboxylase;
c) acetoin to 3-amino-2-butanol, which may be catalyzed, for example, acetonin aminase;
d) 3-amino-2-butanol to 3-amino-2-butanol phosphate, which may be catalyzed, for example, by aminobutanol kinase; and,
e) 3-amino-2-butanol phosphate to 2-butanone, which may be catalyzed, for example, by aminobutanol phosphate phosphorylase.

In another embodiment, the engineered 2-butanone biosynthetic pathway comprises the following substrate to product conversions:
a) pyruvate to α-acetolactate, which may be catalyzed, for example, by acetolactate synthase;
b) α-acetolactate to acetoin which may be catalyzed, for example, by acetolactate decarboxylase;
c) acetoin to 2,3-butanediol, which may be catalyzed, for example, by butanediol dehydrogenase;
d) 2,3-butanediol to 2-butanone, which may be catalyzed, for example, by diol dehydratase.

In one embodiment, the invention produces butanol from plant derived carbon sources, avoiding the negative environmental impact associated with standard petrochemical processes for butanol production. In one embodiment, the invention provides a method for the production of butanol using recombinant host cells comprising an engineered butanol pathway.

In some embodiments, the engineered butanol biosynthetic pathway comprises at least one polynucleotide, at least two polynucleotides, at least three polynucleotides, or at least four polynucleotides that is/are heterologous to the host cell. In embodiments, each substrate to product conversion of an engineered butanol biosynthetic pathway in a recombinant host cell is catalyzed by a heterologous polypeptide. In embodiments, the polypeptide catalyzing the substrate to product conversions of acetolactate to 2,3-dihydroxyisovalerate and/or the polypeptide catalyzing the substrate to product conversion of isobutyraldehyde to isobutanol are capable of utilizing NADH as a cofactor.

The terms “acetohydroxyacid synthase,” “acetolactate synthase” and “acetolactate synthetase” (abbreviated “ALS”) are used interchangeably herein to refer to an enzyme that catalyzes the conversion of pyruvate to acetolactate and CO₂. Example acetolactate synthases are known by the EC number 2.2.1.6 (Enzyme Nomenclature 1992, Academic Press, San Diego). These unmodified enzymes are available from a number of sources, including, but not limited to, Bacillus subtilis (GenBank Nos: CAB15618 (SEQ ID NO: 64), Z99122), Klebsiella pneumoniae (GenBank Nos: AAA25079, M73842), and Lactococcus lactis (GenBank Nos: AAA25161, L16975).

The term “ketol-acid reductoisomerase” (“KARI”), and “acetohydroxy acid isomeroreductase” will be used interchangeably and refer to enzymes capable of catalyzing the reaction of (S)-acetolactate to 2,3-dihydroxyisovalerate. Example KARI enzymes may be classified as EC number EC 1.1.1.86 (Enzyme Nomenclature 1992, Academic Press, San Diego), and are available from a vast array of microorganisms, including, but not limited to, Escherichia coli (GenBank Nos: NP_418222, NC 000913), Saccharomyces cerevisiae (GenBank Nos: NP_013459, NM 001182244), Methanococcus maripaludis (GenBank Nos: CAF30210, BX957220), and Bacillus subtilis (GenBank Nos: CAB14789, Z99118). KARIs include Anaerostipes caccae KARI variants “K9G9” and “K9D3” (SEQ ID NOs: 65 and 66, respectively). Ketol-acid reductoisomerase (KARI) enzymes are described in U.S. Patent Appl. Pub. Nos. 2008/0261230 A1, 2009/0163376 A1, 2010/0197519 A1, and PCT Appl. Pub. No. WO 2011/041415, which are incorporated herein by reference. Examples of KARIs disclosed therein are those from Lactococcus lactis, Vibrio cholera, Pseudomonas aeruginosa PAO1, and Pseudomonas fluorescens PF5 variants (SEQ ID NO: 67). In some embodiments, the KARI utilizes NADH. In some embodiments, the KARI utilizes NADPH.

In addition, suitable KARI enzymes include proteins that match the KARI Profile HMM with an E value of <10⁻³ using hmmsearch program in the HMMER package. The theory behind profile HMMs is described in R. Durbin, S. Eddy, A. Krogh, and G. Mitchison, Biological sequence analysis: probabilistic models of proteins and nucleic acids, Cambridge University Press, 1998; Krogh et al., J. Mol. Biol. 235: 1501-1531, 1994. A KARI Profile HMM generated from the alignment of the twenty-five KARIs with experimentally verified function is provided in U.S. Patent Appl. Pub. No. 2011/0313206, which is incorporated herein by reference. Further, KARI enzymes that are a member of a Glade identified through molecular phylogenetic analysis called the SLSL Glade are described in U.S. Patent Appl. Pub. No. 2011/0244536, incorporated herein by reference.

The term “acetohydroxy acid dehydratase” and “dihydroxyacid dehydratase” (“DHAD”) refers to an enzyme that catalyzes the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, E. coli (GenBank Nos: YP_026248, NC_000913), S. cerevisiae (GenBank Nos: NP_012550, NM 001181674), M. maripaludis (GenBank Nos: CAF29874, BX957219), B. subtilis (GenBank Nos: CAB14105, Z99115), L. lactis, and N. crassa. U.S. Patent Appl. Pub. No. 2010/0081154, and U.S. Pat. No. 7,851,188, which are incorporated herein by reference, describe dihydroxyacid dehydratases (DHADs), including a DHAD from Streptococcus mutans (SEQ ID NO: 68).

The term “branched-chain α-keto acid decarboxylase” or “α-ketoacid decarboxylase” or “α-ketoisovalerate decarboxylase” or “2-ketoisovalerate decarboxylase” (“KIVD”) refers to an enzyme that catalyzes the conversion of α-ketoisovalerate to isobutyraldehyde and CO₂. Example branched-chain α-keto acid decarboxylases are known by the EC number 4.1.1.72 and are available from a number of sources, including, but not limited to, Lactococcus lactis (GenBank Nos: AAS49166, AY548760; CAG34226, AJ746364), Salmonella typhimurium (GenBank Nos: NP_461346, NC_003197), Clostridium acetobutylicum (GenBank Nos: NP_149189, NC_001988), M. caseolyticus (SEQ ID NO: 69), and L. grayi (SEQ ID NO: 70).

The term “alcohol dehydrogenase” (“ADH”) refers to an enzyme that catalyzes the conversion of isobutyraldehyde to isobutanol, 2-butanone to 2-butanol, and/or butyraldehyde to 1-butanol. Alcohol dehydrogenases may be “branched chain alcohol dehydrogenases” or may be referred to as “butanol dehydrogenases.” Example alcohol dehydrogenases suitable for embodiments disclosed herein may be known by the EC number 1.1.1.265, but may also be classified under other alcohol dehydrogenases, for example, according to published utilization of NADH (typically 1.1.1.1) or NADPH (typically 1.1.1.2) as cofactors. Such enzymes are available from a number of sources, including, but not limited to, S. cerevisiae (GenBank Nos: NP_010656; NC_001136; NP_014051; NC_001145); E. coli (GenBank Nos: NP_417484; NC_000913), C. acetobutylicum (GenBank Nos: NP_349892, NC_003030; NP_349891, NC_003030; NP_149325, NC_001988), Pyrococcus furiosus (GenBank Nos: AAC25556, AF013169), Acinetobacter sp. (GenBank Nos: AAG10026, AF282240), Rhodococcus ruber (GenBank Nos: CAD36475, AJ491307), Achromobacter xylosoxidans (SEQ ID NO: 71), and Beijerinkia indica (SEQ ID NO: 72).

The term “branched-chain keto acid dehydrogenase” refers to an enzyme that catalyzes the conversion of α-ketoisovalerate to isobutyryl-CoA (isobutyryl-coenzyme A), typically using NAD⁺ (nicotinamide adenine dinucleotide) as an electron acceptor. Example branched-chain keto acid dehydrogenases are known by the EC number 1.2.4.4. Such branched-chain keto acid dehydrogenases are comprised of four subunits and sequences from all subunits are available from a vast array of microorganisms, including, but not limited to, B. subtilis (GenBank Nos: CAB14336, Z99116; CAB14335, Z99116; CAB14334, Z99116; and CAB14337, Z99116) and Pseudomonas putida (GenBank Nos: AAA65614, M57613; AAA65615, M57613; AAA65617), M57613); and AAA65618, M57613).

The term “acylating aldehyde dehydrogenase” refers to an enzyme that catalyzes the conversion of isobutyryl-CoA to isobutyraldehyde, typically using either NADH or NADPH as an electron donor. Example acylating aldehyde dehydrogenases are known by the EC numbers 1.2.1.10 and 1.2.1.57. Such enzymes are available from multiple sources, including, but not limited to, Clostridium beijerinckii (GenBank Nos: AAD31841, AF157306), C. acetobutylicum (GenBank Nos: NP_149325, NC_001988; NP_149199, NC_001988), P. putida (GenBank Nos: AAA89106, U13232), and Thermus thermophilus (GenBank Nos: YP_145486, NC_006461).

The term “transaminase” refers to an enzyme that catalyzes the conversion of α-ketoisovalerate to L-valine, using either alanine or glutamate as an amine donor. Example transaminases are known by the EC numbers 2.6.1.42 and 2.6.1.66. Such enzymes are available from a number of sources. Examples of sources for alanine-dependent enzymes include, but are not limited to, E. coli (GenBank Nos: YP_026231, NC_000913) and Bacillus licheniformis (GenBank Nos: YP_093743, NC_006322). Examples of sources for glutamate-dependent enzymes include, but are not limited to, E. coli (GenBank Nos: YP_026247, NC_000913), S. cerevisiae (GenBank Nos: NP_012682, NC_001142) and Methanobacterium thermoautotrophicum (GenBank Nos: NP_276546, NC_000916).

The term “valine dehydrogenase” refers to an enzyme that catalyzes the conversion of α-ketoisovalerate to L-valine, typically using NAD(P)H as an electron donor and ammonia as an amine donor. Example valine dehydrogenases are known by the EC numbers 1.4.1.8 and 1.4.1.9 and such enzymes are available from a number of sources, including, but not limited to, Streptomyces coelicolor (GenBank Nos: NP_628270, NC_003888) and B. subtilis (GenBank Nos: CAB14339, Z99116).

The term “valine decarboxylase” refers to an enzyme that catalyzes the conversion of L-valine to isobutylamine and CO₂. Example valine decarboxylases are known by the EC number 4.1.1.14. Such enzymes are found in Streptomyces, such as for example, Streptomyces viridifaciens (GenBank Nos: AAN10242, AY116644).

The term “omega transaminase” refers to an enzyme that catalyzes the conversion of isobutylamine to isobutyraldehyde using a suitable amino acid as an amine donor. Example omega transaminases are known by the EC number 2.6.1.18 and are available from a number of sources, including, but not limited to, Alcaligenes denitrificans (AAP92672, AY330220), Ralstonia eutropha (GenBank Nos: YP_294474, NC_007347), Shewanella oneidensis (GenBank Nos: NP_719046, NC_004347), and P. putida (GenBank Nos: AAN66223, AE016776).

The term “acetyl-CoA acetyltransferase” refers to an enzyme that catalyzes the conversion of two molecules of acetyl-CoA to acetoacetyl-CoA and coenzyme A (CoA). Example acetyl-CoA acetyltransferases are acetyl-CoA acetyltransferases with substrate preferences (reaction in the forward direction) for a short chain acyl-CoA and acetyl-CoA and are classified as E.C. 2.3.1.9 [Enzyme Nomenclature 1992, Academic Press, San Diego]; although, enzymes with a broader substrate range (E.C. 2.3.1.16) will be functional as well. Acetyl-CoA acetyltransferases are available from a number of sources, for example, Escherichia coli (GenBank Nos: NP_416728, NC_000913; NCBI (National Center for Biotechnology Information) amino acid sequence, NCBI nucleotide sequence), Clostridium acetobutylicum (GenBank Nos: NP_349476.1, NC_003030; NP_149242, NC_001988, Bacillus subtilis (GenBank Nos: NP_390297, NC_000964), and Saccharomyces cerevisiae (GenBank Nos: NP_015297, NC_001148).

The term “3-hydroxybutyryl-CoA dehydrogenase” refers to an enzyme that catalyzes the conversion of acetoacetyl-CoA to 3-hydroxybutyryl-CoA. 3-Example hydroxybutyryl-CoA dehydrogenases may be reduced nicotinamide adenine dinucleotide (NADH)-dependent, with a substrate preference for (S)-3-hydroxybutyryl-CoA or (R)-3-hydroxybutyryl-CoA. Examples may be classified as E.C. 1.1.1.35 and E.C. 1.1.1.30, respectively. Additionally, 3-hydroxybutyryl-CoA dehydrogenases may be reduced nicotinamide adenine dinucleotide phosphate (NADPH)-dependent, with a substrate preference for (S)-3-hydroxybutyryl-CoA or (R)-3-hydroxybutyryl-CoA and are classified as E.C. 1.1.1.157 and E.C. 1.1.1.36, respectively. 3-Hydroxybutyryl-CoA dehydrogenases are available from a number of sources, for example, C. acetobutylicum (GenBank NOs: NP_349314, NC_003030), B. subtilis (GenBank NOs: AAB09614, U29084), Ralstonia eutropha (GenBank NOs: YP_294481, NC_007347), and Alcaligenes eutrophus (GenBank NOs: AAA21973, J04987).

The term “crotonase” refers to an enzyme that catalyzes the conversion of 3-hydroxybutyryl-CoA to crotonyl-CoA and H₂O. Example crotonases may have a substrate preference for (S)-3-hydroxybutyryl-CoA or (R)-3-hydroxybutyryl-CoA and may be classified as E.C. 4.2.1.17 and E.C. 4.2.1.55, respectively. Crotonases are available from a number of sources, for example, E. coli (GenBank NOs: NP_415911, NC_000913), C. acetobutylicum (GenBank NOs: NP_349318, NC_003030), B. subtilis (GenBank NOs: CAB13705, Z99113), and Aeromonas caviae (GenBank NOs: BAA21816, D88825).

The term “butyryl-CoA dehydrogenase” refers to an enzyme that catalyzes the conversion of crotonyl-CoA to butyryl-CoA. Example butyryl-CoA dehydrogenases may be NADH-dependent, NADPH-dependent, or flavin-dependent and may be classified as E.C. 1.3.1.44, E.C. 1.3.1.38, and E.C. 1.3.99.2, respectively. Butyryl-CoA dehydrogenases are available from a number of sources, for example, C. acetobutylicum (GenBank NOs: NP_347102, NC_003030), Euglena gracilis (GenBank NOs: Q5EU90), AY741582), Streptomyces collinus (GenBank NOs: AAA92890, U37135), and Streptomyces coelicolor (GenBank NOs: CAA22721, AL939127).

The term “butyraldehyde dehydrogenase” refers to an enzyme that catalyzes the conversion of butyryl-CoA to butyraldehyde, and may use NADH or NADPH as cofactor. Example butyraldehyde dehydrogenases with a preference for NADH may be known as E.C. 1.2.1.57 and are available from, for example, Clostridium beijerinckii (GenBank NOs: AAD31841, AF157306) and C. acetobutylicum (GenBank NOs: NP_149325, NC_001988).

The term “isobutyryl-CoA mutase” refers to an enzyme that catalyzes the conversion of butyryl-CoA to isobutyryl-CoA. This enzyme may use coenzyme B₁₂ as cofactor. Example isobutyryl-CoA mutases are known by the EC number 5.4.99.13. These enzymes are found in a number of Streptomyces, including, but not limited to, Streptomyces cinnamonensis (GenBank Nos: AAC08713, U67612; CAB59633, AJ246005), S. coelicolor (GenBank Nos: CAB70645, AL939123; CAB92663, AL939121), and Streptomyces avermitilis (GenBank Nos: NP_824008, NC_003155; NP_824637, NC_003155).

The term “acetolactate decarboxylase” refers to a polypeptide (or polypeptides) having an enzyme activity that catalyzes the conversion of alpha-acetolactate to acetoin. Example acetolactate decarboxylases may be known as EC 4.1.1.5 and are available, for example, from Bacillus subtilis (GenBank Nos: AAA22223, L04470), Klebsiella terrigena (GenBank Nos: AAA25054, L04507) and Klebsiella pneumoniae (GenBank Nos: AAU43774, AY722056).

The term “acetoin aminase” or “acetoin transaminase” refers to a polypeptide (or polypeptides) having an enzyme activity that catalyzes the conversion of acetoin to 3-amino-2-butanol. Acetoin aminase may utilize the cofactor pyridoxal 5′-phosphate or NADH (reduced nicotinamide adenine dinucleotide) or NADPH (reduced nicotinamide adenine dinucleotide phosphate). The resulting product may have (R) or (S) stereochemistry at the 3-position. The pyridoxal phosphate-dependent enzyme may use an amino acid such as alanine or glutamate as the amino donor. The NADH- and NADPH-dependent enzymes may use ammonia as a second substrate. A suitable example of an NADH dependent acetoin aminase, also known as amino alcohol dehydrogenase, is described by Ito et al. (U.S. Pat. No. 6,432,688). An example of a pyridoxal-dependent acetoin aminase is the amine:pyruvate aminotransferase (also called amine:pyruvate transaminase) described by Shin and Kim (J. Org. Chem. 67:2848-2853 (2002)).

The term “acetoin kinase” refers to a polypeptide (or polypeptides) having an enzyme activity that catalyzes the conversion of acetoin to phosphoacetoin. Acetoin kinase may utilize ATP (adenosine triphosphate) or phosphoenolpyruvate as the phosphate donor in the reaction. Example enzymes that catalyze the analogous reaction on the similar substrate dihydroxyacetone, for example, include enzymes known as EC 2.7.1.29 (Garcia-Alles et al. (2004) Biochemistry 43:13037-13046).

The term “acetoin phosphate aminase” refers to a polypeptide (or polypeptides) having an enzyme activity that catalyzes the conversion of phosphoacetoin to 3-amino-2-butanol O-phosphate. Acetoin phosphate aminase may use the cofactor pyridoxal 5′-phosphate, NADH or NADPH. The resulting product may have (R) or (S) stereochemistry at the 3-position. The pyridoxal phosphate-dependent enzyme may use an amino acid such as alanine or glutamate. The NADH and NADPH-dependent enzymes may use ammonia as a second substrate. Although there are no reports of enzymes catalyzing this reaction on phosphoacetoin, there is a pyridoxal phosphate-dependent enzyme that is proposed to carry out the analogous reaction on the similar substrate serinol phosphate (Yasuta et al. (2001) Appl. Environ. Microbial. 67:4999-5009).

The term “aminobutanol phosphate phospholyase”, also called “amino alcohol 0-phosphate lyase”, refers to a polypeptide (or polypeptides) having an enzyme activity that catalyzes the conversion of 3-amino-2-butanol 0-phosphate to 2-butanone. Amino butanol phosphate phospholyase may utilize the cofactor pyridoxal 5′-phosphate. There are reports of enzymes that catalyze the analogous reaction on the similar substrate 1-amino-2-propanol phosphate (Jones et al. (1973) Biochem J. 134:167-182). U.S. Patent Appl. Pub. No. 2007/0259410 describes an aminobutanol phosphate phospholyase from the organism Erwinia carotovora.

The term “aminobutanol kinase” refers to a polypeptide (or polypeptides) having an enzyme activity that catalyzes the conversion of 3-amino-2-butanol to 3-amino-2butanol O-phosphate. Amino butanol kinase may utilize ATP as the phosphate donor. Although there are no reports of enzymes catalyzing this reaction on 3-amino-2-butanol, there are reports of enzymes that catalyze the analogous reaction on the similar substrates ethanolamine and 1-amino-2-propanol (Jones et al., supra). U.S. Patent Appl. Pub. No. 2009/0155870 describes, in Example 14, an amino alcohol kinase of Erwinia carotovora subsp. Atroseptica.

The term “butanediol dehydrogenase” also known as “acetoin reductase” refers to a polypeptide (or polypeptides) having an enzyme activity that catalyzes the conversion of acetoin to 2,3-butanediol. Butanediol dehydrogenases are a subset of the broad family of alcohol dehydrogenases. Butanediol dehydrogenase enzymes may have specificity for production of (R)- or (S)-stereochemistry in the alcohol product. Example (S)-specific butanediol dehydrogenases may be known as EC 1.1.1.76 and are available, for example, from Klebsiella pneumoniae (GenBank Nos: BBA13085, D86412). Example (R)-specific butanediol dehydrogenases may be known as EC 1.1.1.4 and are available, for example, from Bacillus cereus (GenBank Nos. NP 830481, NC_004722; AAP07682, AE017000), and Lactococcus lactis (GenBank Nos. AAK04995, AE006323).

The term “butanediol dehydratase”, also known as “diol dehydratase” or “propanediol dehydratase” refers to a polypeptide (or polypeptides) having an enzyme activity that catalyzes the conversion of 2,3-butanediol to 2-butanone. Example butanediol dehydratase may utilize the cofactor adenosyl cobalamin (also known as coenzyme B12 or vitamin B12; although vitamin B12 may refer also to other forms of cobalamin that are not coenzyme B12). Example adenosyl cobalamin-dependent enzymes may be known as EC 4.2.1.28 and are available, for example, from Klebsiella oxytoca (GenBank Nos: AA08099 (alpha subunit), D45071; BAA08100 (beta subunit), D45071; and BBA08101 (gamma subunit), D45071 (Note all three subunits are required for activity)], and Klebsiella pneumonia (GenBank Nos: AAC98384 (alpha subunit), AF102064; GenBank Nos: AAC98385 (beta subunit), AF102064, GenBank Nos: AAC98386 (gamma subunit), AF102064). Other suitable diol dehydratases include, but are not limited to, B12-dependent diol dehydratases available from Salmonella typhimurium (GenBank Nos: AAB84102 (large subunit), AF026270; GenBank Nos: AAB84103 (medium subunit), AF026270; GenBank Nos: AAB84104 (small subunit), AF026270); and Lactobacillus collinoides (GenBank Nos: CAC82541 (large subunit), AJ297723; GenBank Nos: CAC82542 (medium subunit); AJ297723; GenBank Nos: CAD01091 (small subunit), AJ297723); and enzymes from Lactobacillus brevis (particularly strains CNRZ 734 and CNRZ 735, Speranza et al., J. Agric. Food Chem. (1997) 45:3476-3480), and nucleotide sequences that encode the corresponding enzymes. Methods of diol dehydratase gene isolation are well known in the art (e.g., U.S. Pat. No. 5,686,276).

It will be appreciated that host cells comprising an engineered butanol biosynthetic pathway as provided herein may further comprise one or more additional modifications. In some embodiments, host cells contain a deletion or downregulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase. In some embodiments, the host cells comprise modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Patent Appl. Pub. No. 2009/0305363 (incorporated herein by reference). In some embodiments, the host cells comprise modifications that provide for increased carbon flux through an Entner-Doudoroff Pathway or reducing equivalents balance as described in U.S. Patent Appl. Pub. No. 2010/0120105 (incorporated herein by reference). Other modifications include integration of at least one polynucleotide encoding a polypeptide that catalyzes a step in a pyruvate-utilizing biosynthetic pathway. Other modifications include at least one deletion, mutation, and/or substitution in an endogenous polynucleotide encoding a polypeptide having acetolactate reductase activity as described in PCT Publication No. WO 2011/159853 (incorporated herein by reference). In embodiments, the polypeptide having acetolactate reductase activity is YMR226C (SEQ ID NOs: 73) of Saccharomyces cerevisiae or a homolog thereof. Additional modifications include a deletion, mutation, and/or substitution in an endogenous polynucleotide encoding a polypeptide having aldehyde dehydrogenase and/or aldehyde oxidase activity as described in PCT Publication No. WO 2011/159853 (incorporated herein by reference). In embodiments, the polypeptide having aldehyde dehydrogenase activity is ALD6 from Saccharomyces cerevisiae (SEQ ID NO: 74) or a homolog thereof.

Recombinant host cells may further comprise (a) at least one heterologous polynucleotide encoding a polypeptide having dihydroxy-acid dehydratase activity; and (b)(i) at least one deletion, mutation, and/or substitution in an endogenous gene encoding a polypeptide affecting Fe—S cluster biosynthesis; and/or (ii) at least one heterologous polynucleotide encoding a polypeptide affecting Fe—S cluster biosynthesis. In embodiments, the polypeptide affecting Fe—S cluster biosynthesis is encoded by AFT1, AFT2, CCC1, FRA2, or GRX3. AFT1 and AFT2 are described in WO 2001/103300, which is incorporated herein by reference. In embodiments, the polypeptide affecting Fe—S cluster biosynthesis is constitutive mutant AFT1 L99A, AFT1 L102A, AFT1 C291F, or AFT1 C293F.

### Butanol Production

Disclosed herein are processes suitable for production of butanol from a carbon substrate and employing a microorganism. In some embodiments, microorganisms may comprise an engineered butanol biosynthetic pathway, such as, but not limited to engineered isobutanol biosynthetic pathways disclosed elsewhere herein. The ability to utilize carbon substrates to produce isobutanol can be confirmed using methods known in the art, including, but not limited to those described in U.S. Pat. No. 7,851,188, which is incorporated herein by reference. For example, a specific high performance liquid chromatography (HPLC) method utilized a Shodex SH-1011 column with a Shodex SH-G guard column, both purchased from Waters Corporation (Milford, Mass.), with refractive index (RI) detection. Chromatographic separation was achieved using 0.01 M H₂SO₄ as the mobile phase with a flow rate of 0.5 mL/min and a column temperature of 50° C. Isobutanol had a retention time of 46.6 min under the conditions used. Alternatively, gas chromatography (GC) methods are available. For example, a specific GC method utilized an HP-INNOWax column (30 m×0.53 mm id, 1 μm film thickness, Agilent Technologies, Wilmington, Del.), with a flame ionization detector (FID). The carrier gas was helium at a flow rate of 4.5 mL/min, measured at 150° C. with constant head pressure; injector split was 1:25 at 200° C.; oven temperature was 45° C. for 1 min, 45 to 220° C. at 10° C./min, and 220° C. for 5 min; and FID detection was employed at 240° C. with 26 mL/min helium makeup gas. The retention time of isobutanol was 4.5 min.

One embodiment of the invention is directed to a microorganism comprising a pyruvate utilizing biosynthetic pathway, wherein the microorganism further comprises reduced pyruvate decarboxylase activity and modified adenylate cyclase activity. In a further embodiment, the pyruvate utilizing biosynthetic pathway is an engineered butanol production pathway. In some embodiments, the engineered butanol production pathway is an engineered isobutanol production pathway

In some embodiments, the engineered isobutanol production pathway comprises the following substrate to product conversions: (a) pyruvate to acetolactate; (b) acetolactate to 2,3-dihydroxyisovalerate; (c) 2,3-dihydroxyisovalerate to α-ketoisovalerate; (d) α-ketoisovalerate to isobutyraldehyde, and (e) isobutyraldehyde to isobutanol.

In some embodiments, the microorganism is a member of a genus of Saccharomyces, Schizosaccharomyces, Hansenula, Candida, Kluyveromyces, Yarrowia, Issatchenkia, or Pichia. In some embodiments, the microorganism is Saccharomyces cerevisiae.

In some embodiments, the engineered microorganism contains one or more polypeptides selected from a group of enzymes having the following Enzyme Commission Numbers: EC 2.2.1.6, EC 1.1.1.86, EC 4.2.1.9, EC 4.1.1.72, EC 1.1.1.1, EC 1.1.1.265, EC 1.1.1.2, EC 1.2.4.4, EC 1.3.99.2, EC 1.2.1.57, EC 1.2.1.10, EC 2.6.1.66, EC 2.6.1.42, EC 1.4.1.9, EC 1.4.1.8, EC 4.1.1.14, EC 2.6.1.18, EC 2.3.1.9, EC 2.3.1.16, EC 1.1.130, EC 1.1.1.35, EC 1.1.1.157, EC 1.1.1.36, EC 4.2.1.17, EC 4.2.1.55, EC 1.3.1.44, EC 1.3.1.38, EC 5.4.99.13, EC 4.1.1.5, EC 2.7.1.29, EC 1.1.1.76, EC 1.2.1.57, and EC 4.2.1.28.

In some embodiments, the engineered microorganism contains one or more polypeptides selected from acetolactate synthase, acetohydroxy acid isomeroreductase, acetohydroxy acid dehydratase, branched-chain alpha-keto acid decarboxylase, branched-chain alcohol dehydrogenase, acylating aldehyde dehydrogenase, branched-chain keto acid dehydrogenase, butyryl-CoA dehydrogenase, butyraldehyde dehydrogenase, transaminase, valine dehydrogenase, valine decarboxylase, omega transaminase, acetyl-CoA acetyltransferase, 3-hydroxybutyryl-CoA dehydrogenase, crotonase, butyryl-CoA dehydrogenase, isobutyryl-CoA mutase, acetolactate decarboxylase, acetonin aminase, butanol dehydrogenase, butyraldehyde dehydrogenase, acetoin kinase, acetoin phosphate aminase, aminobutanol phosphate phospholyase, aminobutanol kinase, butanediol dehydrogenase, and butanediol dehydratase.

In some embodiments, the engineered microorganism contains a polypeptide selected using a KARI Profile HMM. A KARI Profile Hidden Markov Model (HMM) generated from the alignment of the twenty-five KARIs with experimentally verified function is given in U.S. Patent Appl. Pub. No. 2011/0313206, incorporated herein by reference. Suitable KARI enzymes include proteins that match the KARI Profile HMM with an E value of <10⁻³ using HMMER 2.2 g hmmsearch program in the HMMER 2.2 g package with the Z parameter set to 1 billion, wherein the Profile HMM for KARIs was built using the HMMER 2.2 g hmmbuild program from a Clustal W alignment using default parameters of the twenty-five KARIs with experimentally verified function and calibrated using the HMMER 2.2 g hmmcalibrate program. The theory behind profile HMMs is described in R. Durbin, S. Eddy, A. Krogh, and G. Mitchison, Biological sequence analysis: probabilistic models of proteins and nucleic acids, Cambridge University Press, 1998; Krogh et al., J. Mol. Biol. 235: 1501-1531, 1994. Further, KARI enzymes that are a member of a Glade identified through molecular phylogenetic analysis called the SLSL Glade are described in U.S. Patent Appl. Pub. No. 2011/0244536, incorporated herein by reference. Additional suitable KARI enzymes are described in U.S. Patent Appl. Pub. Nos. 2008/0261230, 2009/0163376, and 2010/0197519, each incorporated herein by reference.

In some embodiments, the carbon substrate is selected from the group consisting of: oligosaccharides, polysaccharides, monosaccharides, and mixtures thereof. In some embodiments, the carbon substrate is selected from the group consisting of: fructose, glucose, lactose, maltose, galactose, sucrose, starch, cellulose, feedstocks, ethanol, lactate, succinate, glycerol, corn mash, sugar cane, biomass, a C5 sugar, such as xylose and arabinose, and mixtures thereof.

In some embodiments, one or more of the substrate to product conversions utilizes NADH or NADPH as a cofactor.

In some embodiments, enzymes from the biosynthetic pathway are localized to the cytosol. In some embodiments, enzymes from the biosynthetic pathway that are usually localized to the mitochondria are localized to the cytosol. In some embodiments, an enzyme from the biosynthetic pathway is localized to the cytosol by removing the mitochondrial targeting sequence. In some embodiments, mitochondrial targeting is eliminated by generating new start codons as described in e.g., U.S. Pat. No. 7,851,188, which is incorporated herein by reference in its entirety. In some embodiments, the enzyme from the biosynthetic pathway that is localized to the cytosol is DHAD. In some embodiments, the enzyme from the biosynthetic pathway that is localized to the cytosol is KARI.

In some embodiments, microorganisms are contacted with carbon substrates under conditions whereby a fermentation product is produced. In some embodiments, the fermentation product is butanol. In some embodiments, the butanol is isobutanol.

In some embodiments, the butanologen produces butanol at least 90% of theoretical yield, at least 91% of theoretical yield, at least 92% of theoretical yield, at least 93% of theoretical yield, at least 94% of theoretical yield, at least 95% of theoretical yield, at least 96% of theoretical yield, at least 97% of theoretical yield, at least 98% of theoretical yield, or at least 99% of theoretical yield. In some embodiments, the butanologen produces butanol at least 55% to at least 75% of theoretical yield, at least 50% to at least 80% of theoretical yield, at least 45% to at least 85% of theoretical yield, at least 40% to at least 90% of theoretical yield, at least 35% to at least 95% of theoretical yield, at least 30% to at least 99% of theoretical yield, at least 25% to at least 99% of theoretical yield, at least 10% to at least 99% of theoretical yield or at least 10% to 100% of theoretical yield.

Microorganisms

In embodiments, suitable microorganisms include any microorganism useful for genetic modification and recombinant gene expression and that is capable of producing a C3-C6 alcohol by fermentation. In other embodiments, the microorganism is a butanologen. In other embodiments, the butanologen is a yeast host cell. In other embodiments, the yeast host cell can be a member of the genera Schizosaccharomyces, Issatchenkia, Kluyveromyces, Yarrowia, Pichia, Candida, Hansenula, or Saccharomyces. In other embodiments, the host cell can be Saccharomyces cerevisiae, Schizosaccharomyces pombe, Kluyveromyces lactis, Kluyveromyces thermotolerans, Kluyveromyces marxianus, Candida glabrata, Candida albicans, Pichia shpitis, or Yarrowia hpolytica. In some embodiments, the host cell is a member of the genera Saccharomyces. In some embodiments, the host cell is Kluyveromyces lactis, Candida glabrata or Schizosaccharomyces pombe. In some embodiments, the host cell is Saccharomyces cerevisiae. S. cerevisiae yeast are known in the art and are available from a variety of sources, including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae include, but are not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

In some embodiments the microorganism is a diploid cell. In a further embodiment the organism is a MATa/MATa diploid, a MATa/MATa diploid, or a MATa/MATa diploid. In some embodiments the organism is a haploid. In a further embodiment the organism is a MATa haploid or a MATa haploid.

In some embodiments, the microorganism expresses an engineered C3-C6 alcohol production pathway. In some embodiments the microorganism is a butanologen that expresses an engineered butanol biosynthetic pathway. In some embodiments, the butanologen is an isobutanologen expressing an engineered isobutanol biosynthetic pathway.

### Carbon Substrates

Suitable carbon substrates may include, but are not limited to, monosaccharides such as fructose or glucose, oligosaccharides such as lactose, maltose, galactose, or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol.

“Sugar” includes monosaccharides such as fructose or glucose, oligosaccharides such as lactose, maltose, galactose, or sucrose, polysaccharides such as starch or cellulose, C5 sugars such as xylose and arabinose, and mixtures thereof.

Additionally the carbon substrate may also be one-carbon substrates such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth C1 Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, in some embodiments, the carbon substrates are glucose, fructose, and sucrose, or mixtures of these with C5 sugars such as xylose and arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in U.S. Patent Application Publication No. 2007/0031918 A1, which is incorporated herein by reference. Biomass includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In some embodiments, the carbon substrate is glucose derived from corn. In some embodiments, the carbon substrate is glucose derived from wheat. In some embodiments, the carbon substrate is sucrose derived from sugar cane.

In addition to an appropriate carbon source, fermentation media must contain suitable minerals, salts, cofactors, buffers and other components, known to those skilled in the art, suitable for the growth of the cultures and promotion of an enzymatic pathway described herein.

### Fermentation Conditions

Typically cells are grown at a temperature in the range of about 20° C. to about 40° C. in an appropriate medium. Suitable growth media in the present invention include common commercially prepared media such as Sabouraud Dextrose (SD) broth, Yeast Medium (YM) broth, or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between pH 3.0 to pH 7.5, where pH 4.5 to pH 6.5 is preferred as the initial condition. Fermentations may be performed under aerobic or anaerobic conditions, where anaerobic or microaerobic conditions are preferred.

The amount of butanol produced in the fermentation medium can be determined using a number of methods known in the art, for example, high performance liquid chromatography (HPLC) or gas chromatography (GC).

### Industrial Batch and Continuous Fermentations

Isobutanol, or other products, may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992).

Isobutanol, or other products, may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of isobutanol, or other products, may be practiced using batch, fed-batch or continuous processes and that any known mode of fermentation would be suitable. Additionally, it is contemplated that cells may be immobilized on a substrate as whole cell catalysts and subjected to fermentation conditions for isobutanol production.

Methods for Butanol Isolation from the Fermentation Medium

Bioproduced butanol may be isolated from the fermentation medium using methods known in the art for ABE fermentations (see, e.g., Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the isobutanol may be isolated from the fermentation medium using methods such as distillation, azeotropic distillation, liquid-liquid extraction, adsorption, gas stripping, membrane evaporation, or pervaporation.

Because butanol forms a low boiling point, azeotropic mixture with water, distillation can be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the butanol. In this method, the butanol containing fermentation broth is distilled to near the azeotropic composition. Then, the azeotropic mixture is condensed, and the butanol is separated from the fermentation medium by decantation. The decanted aqueous phase may be returned to the first distillation column as reflux. The butanol-rich decanted organic phase may be further purified by distillation in a second distillation column.

The butanol can also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption can also be used to isolate butanol from the fermentation medium. In this method, the fermentation broth containing the butanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the butanol from the fermentation medium. In this method, the fermentation broth containing the butanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 2009/0305370, the disclosure of which is hereby incorporated in its entirety. U.S. Patent Appl. Pub. No. 2009/0305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, an ester can be formed by contacting the alcohol in a fermentation medium with an organic acid (e.g., fatty acids) and a catalyst capable of esterfiying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant. Other butanol product recovery and/or ISPR methods may be employed, including those described in U.S. Pat. No. 8,101,808, incorporated herein by reference.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

Butanol titer in any phase can be determined by methods known in the art, such as via high performance liquid chromatography (HPLC) or gas chromatography, as described, for example, in U.S. Patent Appl. Pub. No. 2009/0305370, which is incorporated herein by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “s” means second(s), “min” means minute(s), “h” means hour(s), “psi” means pounds per square inch, “nm” means nanometers, “d” means day(s), “μL” means microliter(s), “mL” means milliliter(s), “L” means liter(s), “mm” means millimeter(s), “nm” means nanometers, “mM” means millimolar, “μM” means micromolar, “M” means molar, “mmol” means millimole(s), “μmol” means micromole(s)”, “g” means gram(s), “μg” means microgram(s) and “ng” means nanogram(s), “PCR” means polymerase chain reaction, “OD” means optical density, “OD₆₀₀” means the optical density measured at a wavelength of 600 nm, “cfu” means colony forming units, “kDa” means kilodaltons, “g” means the gravitation constant, “bp” means base pair(s), “kb” means kilobase pair(s), “% w/v” means weight/volume percent, % v/v″ means volume/volume percent, “HPLC” means high performance liquid chromatography, and “GC” means gas chromatography

### General Methods

Materials and methods suitable for the maintenance and growth of yeast cultures are well known in the art. Techniques suitable for use in the following Examples may be found as set out in Yeast Protocols, Second Edition (Wei Xiao, ed; Humana Press, Totowa, N.J. (2006))). All reagents were obtained from Aldrich Chemicals (Milwaukee, Wis.), BD Diagnostic Systems (Sparks, Md.), Life Technologies (Rockville, Md.), Sigma Chemical Company (St. Louis, Mo.), or Teknova (Half Moon Bay, Calif.) unless otherwise specified.

YPD contains per liter: 10 g yeast extract, 20 g peptone, and 20 g dextrose. YPE contains per liter: 10 g yeast extract, 20 g peptone, and 1% ethanol. PM contains per liter: 6.7 g yeast nitrogen base without amino acids, 1 g yeast extract, 3 mL nicotinic acid (10 mg/mL), 19.5 g 100 mM MES, 30 g glucose, pH 5.5.

The oligonucleotide primers to use in the following Examples are given in Table 6. All the oligonucleotide primers are synthesized by Sigma-Genosys (Woodlands, Tex.).

The strains referenced in the following Examples are given in Table 5.

TABLE 5
Strains referenced in the Examples

| Strain Name | Genotype | Description |
|---|---|---|
| PNY2145 | ura3Δ::loxP his3Δ pdc5Δ::P[FBA(L8)]- | U.S. Patent Appl. Pub. |
| | XPK\|xpk1_Lp-CYCt-loxP66/71 fra2Δ 2- | No. 2013/0252296, |
| | micron (CEN.PK2) pdc1Δ::P[PDC1]- | incorporated herein by |
| | ALS\|alsS_Bs-CYC1t-loxP71/66 | reference |
| | pdc6Δ::(UAS)PGK1-P[FBA1]-KIVD\|Lg(y)- | |
| | TDH3t-loxP71/66 adh1Δ::P[ADH1]- | |
| | ADH\|Bi(y)-ADHt-loxP71/66 fra2Δ::P[ILV5]- | |
| | BiADH\|Bi(y)-ADHt-loxP71/66 | |
| | gpd2Δ::loxP71/66, pdc5Δ::FBA(L8)- | |
| | xpk1::loxP71/66, amn1Δ::AMN1(y) | |

### Determination of Cell Membrane Fatty Acid Content

Fatty Acid Analysis of Saccharomyces cerevisiae

For fatty acid analysis, cells were collected by centrifugation and lipids were extracted as described in Bligh, E. G. & Dyer, W. J. (Can. J. Biochem. Physiol. 37:911-917 (1959)). Fatty acid methyl esters were prepared by transesterification of the lipid extract with sodium methoxide (Roughan, G., and Nishida I., Arch Biochem Biophys. 276(1):38-46 (1990)) and subsequently analyzed with a Hewlett-Packard 6890 GC fitted with a 30-m×0.25 mm (i.d.) HP-INNOWAX (Hewlett-Packard) column. The oven temperature was from 170° C. (25 min hold) to 185° C. at 3.5° C./min.

For direct base transesterification, yeast cultures (25 mL) were harvested, washed once in distilled water, and dried under vacuum in a Speed-Vac for 5-10 min. Sodium methoxide (100 μl of 1%) was added to the sample, and then the sample was vortexed and rocked for 20 min. After adding 3 drops of 1 M NaCl and 400 μl hexane, the sample was vortexed and spun. The upper layer was removed and analyzed by GC as described above.

### Example 1

### Cloning Heterologous Fatty Acid Desaturases into a Yeast Expression Vector

The present example describes the construction of plasmids for the heterologous expression of Yarrowia lipolytica Δ9 desaturase (Yld9d; SEQ ID NO: 1), Mortierella alpina Δ9 desaturase (Mad9d; SEQ ID NO: 9), and Fusarium moniliforme Δ12 fatty acid desaturase (Fmd12d; SEQ ID NO: 2) in an isobutanologen.

The ORFs of Y. lipolytica Δ9 desaturase (SEQ ID NO: 3), M. alpina Δ9 desaturase (SEQ ID NO: 10), and F. moniliforme Δ12 fatty acid desaturase (SEQ ID NO: 4) were synthesized using S. cerevisiae codon usage by GenScript USA Inc., 860 Centennial Ave., Piscataway, N.J. 08854, USA, with NcoI and NotI restriction sites and cloned into the NcoI and NotI digested vector, pFBA1-413N (SEQ ID NO.: 75), resulting in plasmids pZ18, pZ26, and pZ12, respectively. The heterologous desaturase ORFs are expressed under the control of the S. cerevisiae fructose-biphosphate aldolase gene (EC 4.1.2.13; GenBank No.: X15003; YKL060C; FBA1) promoter (601 bp upstream of the FBA1 ORF), a ‘ctagtgccacc’ sequence containing the Kozak consensus sequence placed between the FBA1 promoter and the heterologous ORF, and the ADH1 terminator.

Transformation of an Isobutanologen with and Expression of Heterologous Fatty Acid Desaturases Using a Yeast Expression Vector

Isobutanologen strain PNY2145 was co-transformed by the lithium acetate method (Methods in Yeast Genetics, 2005, page 113) with 0.5 μg each of pLH804::L2V4 plasmid and an empty vector, pZ18, or pZ12. pLH804::L2V4 (SEQ ID NO.: 76) contains the K9JB4P variant of Anaerostipes caccae ILVC under the control of S. cerevisiae ILV5 promoter, and the L2V4 variant of Streptococcus mutans ILVD, under the control of S. cerevisiae TEF promoter. PNY2145 was constructed from PNY0827, which was deposited at the ATCC under the Budapest Treaty on Sep. 22, 2011 at the American Type Culture Collection, Patent Depository 10801 University Boulevard, Manassas, Va. 20110-2209 and has the patent deposit designation PTA-12105. Construction of PNY2145 is described in U.S. Patent Appl. Pub. No. 2013/0252296, incorporated herein by reference.

Transformants were selected on minimal medium plates containing 2% ethanol as carbon source. Two empty vector transformants (a, b) and four transformants (a-d) each of pZ18 and pZ12 were grown aerobically in PM in 24-well block at 30° C. An aliquot was used to start 5 mL PM cultures in 15 mL screw cap tunes and grown on a rotary drum for 4 days at 30° C. overnight in PM. Remaining aerobic cultures and all anaerobic cultures were harvested and the pellets analyzed for fatty acid composition.

The fatty acid profile of the average of the four independent transformants of each of pZ18 and pZ12 and of the two empty vector transformants were analyzed by GC method to ascertain the proper expression of the desaturases. Cells were harvested, lipid extracted and GC analyzed as described in the General Method section.

The result of the GC analysis are shown in Tables 6 and 7. In the Δ9 desaturase transformants (pZ18), the ratio of C18:1/C16:1 was increased 50% over the control (Table 6) and the Δ9 desaturase (“d9d”) conversion efficiency ((c.e.); [product/substrate+product]*100) was increased from 87% to 93% (Table 7). Similarly in the Δ12 desaturase transformants (pZ12) the level of and C18:2 fatty acids was enhanced (98 fold) (Table 6).

TABLE 6
Total lipid profile of PNY2145 transformed with vector empty vector, Y. lipolytica
Δ9 desaturase gene, or F. moniliforme Δ12 desaturase gene

| | FAC % Total | | | | | | Ratios | | |
|---|---|---|---|---|---|---|---|---|---|
| Strain | C16:0 | C16:1 | C16:2 | C18:0 | C18:1 | C18:2 | C18:1/C16:1 | C18/C16 | unsaturated/saturated |
| Overnight in PM | | | | | | | | | |
| tube, aerobic | | | | | | | | | |
| empty vector | 12 | 41 | 0 | 6 | 41 | 0 | 1.0 | 0.9 | 4.6 |
| pZ18 (Yl Δ9) | 12 | 34 | 0 | 4 | 51 | 0 | 1.5 | 1.2 | 5.4 |
| pZ12 (Fm Δ12) | 13 | 20 | 0 | 11 | 12 | 43 | 0.6 | 2.0 | 3.1 |
| 4 days in PM | | | | | | | | | |
| tube, anaerobic | | | | | | | | | |
| empty vector | 9 | 52 | 0 | 6 | 32 | 0 | 0.6 | 0.6 | 5.6 |
| pZ18 (Yl Δ9) | 9 | 46 | 0 | 6 | 39 | 0 | 0.8 | 0.8 | 5.8 |
| pZ12 (Fm Δ12) | 10 | 38 | 0 | 9 | 19 | 23 | 0.5 | 1.1 | 4.2 |

TABLE 7
Conversion efficiency of isobutanologens expressing Δ9 or Δ12
desaturases

| | d9d c.e. | d9d c.e. | d9d c.e. | |
|---|---|---|---|---|
| Strain | on C16 | on C18 | on total | elo c.e. |
| Overnight in PM tube, aerobic | | | | |
| empty vector | 78 | 87 | 82 | 48 |
| pZ18 (Yl Δ9) | 74 | 93 | 84 | 55 |
| pZ12 (Fm Δ12) | 60 | 83 | 75 | 66 |
| 4 days hrs in PM tube, anaerobic | | | | |
| empty vector | 85 | 85 | 85 | 38 |
| pZ18 (Yl Δ9) | 83 | 87 | 85 | 44 |
| pZ12 (Fm Δ12) | 80 | 82 | 81 | 52 |

### Example 2

### Replacement of the S. cerevisiae OLE1 Gene with Heterologous Yarrowia lipolytica and Mortierella alpina Δ9 Desaturase Genes

The fatty composition of wild-type Yarrowia lipolytica (Zhang et al., Yeast (2012) 29:25-38), which has a sole Δ9 desaturase gene suggests that it has a 2.4 fold preference for 18:0 over 16:0. Therefore to further improve the level of oleic acid, the host OLE1 gene was replaced with FBA1:Yld9d gene by homologous recombination. For this, the PNY2145 strain was transformed with the OLE1Δ::Yld9d/LoxP/URA3 gene/LoxP DNA cassette (SEQ ID NO.: 77) comprised (5′ to 3′) of 51 bp of the nucleotide sequence immediately upstream of the S. cerevisiae OLE1 ORF, the FBA1 promoter, the Y. lipolytica Δ9 desaturase gene (SEQ ID NO: 3), the ADH1 terminator, loxP71 sequence, the URA3 gene, loxP66 sequence, and the 47 bp immediately downstream of the S. cerevisiae OLE1 ORF. URA3 transformants were selected on URA dropout plates and screened by PCR to identify ole1Δ mutant strains, resulting in the identification of strain C19. The C19 strain was transformed with a GAL1:Cre gene in plasmid pJT254 (BP2054.Cre) (SEQ ID NO: 78) containing the HIS gene as the selectable marker, to excise the LoxP flanked URA3 gene. HIS positive transformants were grown without selection and plated on FOA plates to identify the ura- and his-strain, C32. C32 was reconfirmed by PCR to be lacking the host gene, although the size of the PCR product was less than expected in the mutant strain.

The fatty acid profile of the average of four independent transformants of PNY2145 containing either pZ18 and pZ12, two empty vector transformants, and four independent cultures of C32 were analyzed by GC method. Cells were harvested, lipid extracted and GC analyzed as described in the General Method section. The lipid profile of C32 (Table 8) showed that it was similar to the wild type strain. The conversion efficiency is shown Table 9.

TABLE 8
Total lipid profile of PNY2145 transformed with vector empty vector, Y. lipolytica
Δ9 desaturase gene, or F. moniliforme Δ12 desaturase gene and strain C32 in two
different media

| | FAC % Total | | | | | | Ratios | | |
|---|---|---|---|---|---|---|---|---|---|
| Strain | C16:0 | C16:1 | C16:2 | C18:0 | C18:1 | C18:2 | C18:1/C16:1 | C18/C16 | unsaturated/saturated |
| 3% glucose PM | | | | | | | | | |
| empty vector | 12 | 55 | 1 | 4 | 28 | 0 | 0.5 | 0.5 | 5.0 |
| pZ18 (Yl Δ9) | 14 | 49 | 1 | 3 | 34 | 0 | 0.7 | 0.6 | 4.8 |
| pZ12 (Fm Δ12) | 16 | 22 | 23 | 7 | 8 | 25 | 0.3 | 0.6 | 3.4 |
| OLE1Δ::Yld9d | 15 | 49 | 1 | 4 | 31 | 0 | 0.6 | 0.6 | 4.2 |
| (C32) | | | | | | | | | |
| C32 + pZ12 | 20 | 19 | 24 | 7 | 8 | 23 | 0.4 | 0.6 | 2.8 |
| 0.3% glucose PM | | | | | | | | | |
| empty vector | 9 | 52 | 0 | 6 | 32 | 0 | 0.6 | 0.6 | 5.6 |
| pZ18 (Yl Δ9) | 9 | 46 | 0 | 6 | 39 | 0 | 0.8 | 0.8 | 5.8 |
| pZ12 (Fm Δ12) | 10 | 38 | 0 | 9 | 19 | 23 | 0.5 | 1.1 | 4.2 |
| OLE1Δ::Yld9d | 12 | 55 | 1 | r | 28 | 0 | 0.5 | 0.5 | 5.8 |
| (C32) | | | | | | | | | |
| C32 + pZ12 | 18 | 20 | 29 | 4 | 6 | 24 | 0.3 | 0.5 | 3.6 |

TABLE 9
Conversion efficiency of isobutanologens expressing Δ9 or Δ12
desaturases

| | d9d c.e. | d9d c.e. | d9d c.e. | |
|---|---|---|---|---|
| Strain | on C16 | on C18 | on total | elo c.e. |
| 3% glucose PM | | | | |
| empty vector | 82 | 87 | 83 | 32 |
| pZ18 (Yl Δ9) | 78 | 92 | 83 | 37 |
| pZ12 (Fm Δ12) | 74 | 83 | 77 | 39 |
| OLE1Δ::Yld9d (C32) | 77 | 88 | 81 | 36 |
| C32 + pZ12 | 69 | 82 | 74 | 37 |
| 0.3% glucose PM | | | | |
| empty vector | 85 | 91 | 87 | 30 |
| pZ18 (Yl Δ9) | 81 | 95 | 86 | 32 |
| pZ12 (Fm Δ12) | 78 | 87 | 82 | 35 |
| OLE1Δ::Yld9d (C32) | 82 | 91 | 85 | 31 |
| C32 + pZ12 | 74 | 87 | 78 | 34 |

M. alpina Δ9 desaturase (Mad9d; SEQ ID NO.: 11) has been reported to have a higher preference for C18:0 than C16:0 (Wongwathanarat et al., Microbiology (1999), 145:2939-2946). Therefore, the OLE1 ORF was replaced with that of M. alpina Δ9 desaturase, such that M. alpina Δ9 desaturase ORF (SEQ ID NO: 10) was under the control of the OLE1 promoter. For this PNY2145 was transformed with DNA (SEQ ID NO: 79) comprising (5′ to 3′) 200 bp of the nucleotide sequence immediately upstream of the S. cerevisiae OLE1 ORF, the M. alpina Δ9 desaturase gene (SEQ ID NO: 10), the OLE1 terminator, loxP71 sequence, the URA3 gene, loxP66 sequence, and the 200 bp immediately downstream of the S. cerevisiae OLE1 ORF.

The fatty acid profile of the average of two wild-type strains, two OLE1Δ::Yld9d strains (C32), and OLE1Δ::Mad9d strains (C59 and C60) were analyzed by GC method. Cells were harvested, lipid extracted and GC analyzed as described in the General Method section. Table 10 compares the total lipid profiles of the WT OLE1 and the OLE1Δ::Yld9d and OLE1Δ::Mad9d mutants. Mad9d replacement mutants achieved a very high level (66%) of 18:1. Table 11 compares the conversion efficiency of the various strains.

TABLE 10
Total lipid profile of wild-type, OLE1Δ::Yld9d, and OLE1Δ::Mad9d strains

| | FAC % Total | | | | | | Ratios | | |
|---|---|---|---|---|---|---|---|---|---|
| Strain | C16:0 | C16:1 | C16:2 | C18:0 | C18:1 | C18:2 | C18:1/C16:1 | C18/C16 | unsaturated/saturated |
| wild-type | 7 | 65 | 0 | 1 | 28 | 0 | 0.4 | 0.4 | 12.1 |
| OLE1Δ::Yld9d | 9 | 61 | 0 | 1 | 29 | 0 | 0.5 | 0.4 | 9.2 |
| (C19) | | | | | | | | | |
| OLE1Δ::Mad9d | 24 | 10 | 0 | 0 | 66 | 0 | 6.7 | 1.9 | 3.1 |
| (C59) | | | | | | | | | |
| OLE1Δ::Mad9d | 45 | 15 | 0 | 0 | 40 | 0 | 2.8 | 0.7 | 1.2 |
| (C60) | | | | | | | | | |

TABLE 11
Conversion efficiency of wild-type, OLE1Δ::Yld9d, and OLE1Δ::Mad9d
strains

| | d9d c.e. | d9d c.e. | d9d c.e. | |
|---|---|---|---|---|
| Strain | on C16 | on C18 | on total | elo c.e. |
| wild-type | 91 | 97 | 92 | 29 |
| OLE1Δ::Yld9d (C19) | 88 | 96 | 90 | 30 |
| OLE1Δ::Mad9d (C59) | 29 | 100 | 76 | 66 |
| OLE1Δ::Mad9d (C60) | 25 | 100 | 55 | 40 |

Expression of Heterologous Yarrowia lipolytica and Mortierella alpina Δ9 Desaturase Genes in OLE1D::Yld9d Strains

Strain C32 described above was transformed with additional copies of either FBA1:Yld9d (SEQ ID NO: 80) or FBA1:Mad9d (SEQ ID NO: 81) that were integrated into the genome using DNA cassettes using the delta sequences. Cells were harvested, lipid extracted and GC analyzed as described in the General Method section. Table 12 compares the total lipid profiles of the resultant strains. Table 13 compares their conversion efficiencies.

TABLE 12
Total lipid profile of OLE1Δ::Yld9d strain C32 transformed with FBA1:Yld9d or
FBA1:Mad9d gene by delta integration fragment

| | FAC % Total | | | | | | Ratios | | |
|---|---|---|---|---|---|---|---|---|---|
| Strain | C16:0 | C16:1 | C16:2 | C18:0 | C18:1 | C18:2 | C18:1/C16:1 | C18/C16 | unsaturated/saturated |
| C32 | 11 | 60 | 0 | 2 | 27 | 0 | 0.46 | 0.41 | 7.0 |
| FBA:Yld9d | 11 | 56 | 0 | 1 | 31 | 0 | 0.56 | 0.49 | 7.1 |
| FBA:Yld9d | 11 | 56 | 1 | 1 | 31 | 0 | 0.56 | 0.49 | 7.0 |
| FBA:Yld9d | 15 | 63 | 1 | 2 | 20 | 0 | 0.33 | 0.28 | 5.2 |
| FBA:Yld9d | 11 | 56 | 0 | 2 | 31 | 0 | 0.55 | 0.47 | 6.8 |
| FBA:Yld9d | 11 | 56 | 0 | 2 | 31 | 0 | 0.55 | 0.48 | 6.7 |
| FBA:Yld9d (C53) | 11 | 54 | 1 | 1 | 33 | 0 | 0.62 | 0.53 | 7.3 |
| FBA:Yld9d | 12 | 57 | 0 | 1 | 30 | 0 | 0.53 | 0.46 | 6.8 |
| FBA:Yld9d | 11 | 55 | 0 | 2 | 32 | 0 | 0.57 | 0.50 | 7.0 |
| FBA:Yld9d | 11 | 62 | 1 | 2 | 24 | 0 | 0.39 | 0.34 | 6.7 |
| FBA:Yld9d (C54) | 11 | 55 | 0 | 1 | 33 | 0 | 0.60 | 0.52 | 7.2 |
| FBA:Yld9d | 11 | 55 | 1 | 1 | 32 | 0 | 0.59 | 0.51 | 7.2 |
| FBA:Yld9d | 11 | 54 | 0 | 1 | 33 | 0 | 0.60 | 0.52 | 7.1 |
| FBA:Yld9d | 11 | 57 | 0 | 1 | 30 | 0 | 0.54 | 0.47 | 6.8 |
| average | | | | | | | | | |
| FBA:Mad9d | 11 | 53 | 1 | 1 | 35 | 0 | 0.66 | 0.56 | 7.2 |
| FBA:Mad9d | 11 | 55 | 0 | 1 | 33 | 0 | 0.60 | 0.52 | 7.1 |
| FBA:Mad9d | 11 | 53 | 0 | 2 | 33 | 0 | 0.62 | 0.54 | 6.8 |
| FBA:Mad9d (C55) | 8 | 50 | 0 | 2 | 40 | 0 | 0.80 | 0.71 | 9.0 |
| FBA:Mad9d | 10 | 54 | 0 | 1 | 34 | 0 | 0.64 | 0.55 | 7.6 |
| FBA:Mad9d | 11 | 53 | 0 | 1 | 34 | 0 | 0.64 | 0.56 | 7.3 |
| FBA:Mad9d | 11 | 53 | 0 | 2 | 35 | 0 | 0.66 | 0.57 | 7.2 |
| FBA:Mad9d | 11 | 53 | 0 | 2 | 34 | 0 | 0.65 | 0.56 | 7.3 |
| FBA:Mad9d (C56) | 11 | 53 | 0 | 1 | 35 | 0 | 0.67 | 0.57 | 7.4 |
| FBA:Mad9d | 10 | 53 | 0 | 1 | 35 | 0 | 0.65 | 0.57 | 7.5 |
| FBA:Mad9d | 10 | 53 | 0 | 2 | 35 | 0 | 0.66 | 0.57 | 7.4 |
| FBA:Mad9d | 10 | 54 | 0 | 1 | 34 | 0 | 0.63 | 0.55 | 7.4 |
| FBA:Mad9d | 10 | 53 | 0 | 2 | 35 | 0 | 0.66 | 0.57 | 7.4 |
| average | | | | | | | | | |

TABLE 13
Conversion efficiency of wild-type, OLE1Δ::Yld9d, and OLE1Δ::Mad9d
strains

| | d9d | d9d | d9d | |
|---|---|---|---|---|
| | c.e. on | c.e. on | c.e. on | |
| Strain | C16 | C18 | total | elo c.e. |
| C32 | 85 | 94 | 87 | 29 |
| FBA:Yld9d | 84 | 96 | 88 | 33 |
| FBA:Yld9d | 84 | 96 | 88 | 33 |
| FBA:Yld9d | 81 | 93 | 84 | 22 |
| FBA:Yld9d | 83 | 95 | 87 | 32 |
| FBA:Yld9d | 83 | 95 | 87 | 32 |
| FBA:Yld9d (C53) | 83 | 97 | 88 | 35 |
| FBA:Yld9d | 83 | 96 | 87 | 31 |
| FBA:Yld9d | 84 | 95 | 87 | 33 |
| FBA:Yld9d | 85 | 94 | 87 | 25 |
| FBA:Yld9d (C54) | 84 | 96 | 88 | 34 |
| FBA:Yld9d | 84 | 96 | 88 | 34 |
| FBA:Yld9d | 84 | 96 | 88 | 34 |
| FBA:Yld9d average | 83 | 95 | 87 | 32 |
| FBA:Mad9d | 83 | 96 | 88 | 36 |
| FBA:Mad9d | 83 | 96 | 88 | 34 |
| FBA:Mad9d | 83 | 95 | 87 | 35 |
| FBA:Mad9d (C55) | 86 | 96 | 90 | 42 |
| FBA:Mad9d | 84 | 96 | 88 | 36 |
| FBA:Mad9d | 83 | 96 | 88 | 36 |
| FBA:Mad9d | 83 | 96 | 88 | 36 |
| FBA:Mad9d | 84 | 96 | 88 | 36 |
| FBA:Mad9d (C56) | 83 | 96 | 88 | 36 |
| FBA:Mad9d | 84 | 96 | 88 | 36 |
| FBA:Mad9d | 84 | 96 | 88 | 36 |
| FBA:Mad9d | 84 | 96 | 88 | 35 |
| FBA:Mad9d average | 84 | 96 | 88 | 36 |

### Example 3

### Creation of Strains Expressing Fatty Acid Elongases

Fatty acid elongases that convert C16 fatty acids to C18 fatty acids have been identified and isolated from M. alpina (SEQ ID NO.: 16, U.S. Patent Appl. No. 2007/0087420, incorporated herein by reference) and Y. lipolytica (SEQ ID NO.: 15, U.S. Pat. No. 7,932,077, incorporated herein by reference). A Δ9 fatty acid elongase has also been isolated from Euglena gracilis (SEQ ID NO.: 12). To express these enzymes in S. cerevisiae, DNA fragments containing the coding region of the genes, codon optimized for expression in S. cerevisiae, were synthesized and cloned into the vector pFBA-413N (SEQ ID NO.: 13), under the control of the FBA1 promoter by Genscript. The resulting plasmids were named pZ14 (M. alpina), pZ16 (Y. lipolytica) and pZ10 (E. gracilis).

0.5 μg of pZ10, together with 0.5 μg of plasmid pLH804::L2V4 (SEQ ID NO.: 76), which comprises the K9JB4P variant of Anaerostipes caccae ILVC under the control of S. cerevisiae ILV5 promoter, and the L2V4 variant of Streptococcus mutans ILVD, under the control of S. cerevisiae TEF promoter, were used to transform strain PNY2145, using the lithium acetate method (Methods in Yeast Genetics, 2005, page 113). Transformants were grown in minimal medium containing 2% ethanol as carbon source. One transformant was selected and named PNY3741. This strain expresses the codon optimized E. gracilis Δ9 elongase. Similarly, plasmids pZ14 and pZ16 were used in combination with pLH804::L2V4 (SEQ ID NO.: 76) to transform PNY2145. One of each transformant was selected and named PNY3734 (pZ14) and PNY3735 (pZ16). As a control, PNY2145 was also transformed with vector pFBA-413N and pLH804::L2V4. The resulting strain was named PNY3736.

The fatty acid profile of PNY3734, PNY3735 and PNY3736 was analyzed by GC method to ascertain the proper expression of the elongases. Each strain was grown in synthetic minimal medium with 0.3% glucose (0.3% glucose, 0.67% YNB, 0.1 M MES pH 5.5) overnight. 2 mL of the overnight cultures were used to inoculate 25 mL of SD-high glucose medium (3% glucose, 0.67% YNB, 0.1 M MES, pH 5.5) in 125 mL flasks. Cultures were allowed to grow for 24 hrs at 30° C. and 250 rpm. Cells were harvested, lipid extracted and GC analyzed as described in the General Method section.

The result of the GC analysis was shown in Table 14. PNY3734 and PNY3735 cells contained increased levels of C18 fatty acids, especially C18:1. C16 fatty acid content was reduced.

TABLE 14
Lipid profile of strains PNY3734, PNY3735, and PNY3736

| | | | | | C16 | C18 | Unsaturated/ |
|---|---|---|---|---|---|---|---|
| Strain | C16:0 | C16:1 | C18:0 | C18:1 | Total | Total | Saturated |
| PNY3734 | 1.7 | 5.9 | 4.2 | 72.6 | 7.6 | 76.8 | 13.4 |
| PZ14 (M. alpina) | | | | | | | |
| PNY3735 | 8.2 | 40.8 | 4.9 | 36.9 | 49 | 41.8 | 5.9 |
| PZ16 (Y. lipolytica) | | | | | | | |
| PNY3736 | 9.9 | 45.7 | 4.8 | 32.8 | 55.6 | 37.6 | 5.3 |
| empty vector | | | | | | | |

The fatty acid profile of PNY3741 was also measured. As described above, PNY3741 and PNY3736 cells were grown overnight in minimal medium with 0.3% glucose. 2 mL of each culture were used to inoculate 25 mL of SD-high glucose medium in 125 mL flasks. The flasks were tightly capped, and the culture grown for 24 hrs. Cells were harvested and fatty acid profile analyzed as above. The result is shown in Table 15

TABLE 15
Lipid profile of strains PNY3741 and PNY3736

| | | | | | | C16 | C18 | Unsaturated/ |
|---|---|---|---|---|---|---|---|---|
| Strain | C16:0 | C16:1 | C18:0 | C18:1 | C20:1 | Total | Total | Saturated |
| PNY3741pZ10 | 6.3 | 39.6 | 3.4 | 42.9 | 1.1 | 45.9 | 46.3 | 8.6 |
| (E. gracilis) | | | | | | | | |
| PNY3736 | 7.7 | 42 | 4.5 | 33.9 | 0 | 49.7 | 38.4 | 6.3 |
| empty vector | | | | | | | | |

C16 fatty acids were reduced and C18 fatty acid increased. C18:1 increased from 38% to 46%. C20:1 was present at 1.2%, indicating that the Δ9 elongase could use C18:1 as a substrate.

### Example 4

### Growth and Isobutanol Production of PNY3734, PNY3735, PNY3736 and PNY3741

Growth and isobutanol production of strains expressing elongases were evaluated in a test tube assay. PNY3734, PNY3735, PNY3736 cells were inoculated in 5 mL of synthetic complete medium lacking histidine and uracil, with 0.3% glucose as carbon source, in 15 mL test tubes. The cultures were allowed to grow overnight at 30° C. on a rotary drum. The overnight cultures were diluted to OD 0.2 into 5 mL synthetic complete medium lacking histidine and uracil with 3% glucose as carbon source, and 0, 5 or 8 g/L isobutanol, in 15 mL tubes. The cultures were allowed to grow for 5 hours at 30° C. on the roller drum, then placed in an anaerobic chamber and allowed to grow for 19 hrs at 30° C. and 120 rpm. The OD of each culture was measured, and culture samples were analyzed for isobutanol and other metabolites (see General Methods for details).

As shown in Table 16, PNY3734 and PNY3735 reached higher OD and produced more isobutanol than the control strain PNY3736.

TABLE 16
Growth and isobutanol production of PNY3434, PNY3735,
and PNY3736

| | 0 g/L added | | | | | |
|---|---|---|---|---|---|---|
| | isobutanol | | 5 g/L added | | 8 g/L added | |
| | | Iso- | isobutanol | | isobutanol | |
| | | butanol | | Iso-butanol | | Iso-butanol |
| | Final | produced | Final | produced | Final | produced |
| Strain | O.D. | (mM) | O.D. | (mM) | O.D. | (mM) |
| PNY3734 | 1.15 | 54.7 | 0.84 | 37.4 | 0.63 | 12.8 |
| PNY3735 | 1.23 | 73.7 | 0.80 | 46.8 | 0.59 | 11.2 |
| PNY3736 | 0.97 | 45.7 | 0.63 | 27.5 | 0.43 | 0.66 |

PNY3741 and PNY3736 were inoculated in synthetic minimal medium containing 0.3% glucose as carbon source, and grow overnight at 30° C. on a rotary drum. The overnight cultures were diluted to OD 0.2 into 5 mL synthetic minimal medium with 3% glucose as carbon source, and 5 g/L isobutanol, in 15 mL tubes. The cultures were tightly capped allowed to grow for 24 hours at 30° C. on the roller drum. The OD of each culture was measured, and culture samples were analyzed for isobutanol and other metabolites.

As shown in Table 17, PNY3741 culture achieved a higher OD and produced more isobutanol than PNY3736 control.

TABLE 17
Growth and isobutanol production of PNY3736 and PNY3741 in the
presence of 5 g/L isobutanol

| | | mM Isobutanol | | mM Isobutanol |
|---|---|---|---|---|
| | | produced | OD600 | produced |
| Strain | OD600 (24 hr) | (24 hr) | (48 hr) | (48 hr) |
| PNY3736 | 1.04 | 17.0 | 1.35 | 57.6 |
| PNY3741 | 0.98 | 14.2 | 1.65 | 51 |

### Example 5

### Cloning Lactobacillus plantarum Cyclopropane Fatty Acid Synthase ORFs into a Yeast Expression Vector

Coding sequences encoding Lactobacillus plantarum cyclopropane fatty acid synthase 1 (SEQ ID NO.: 10) and Lactobacillus plantarum cyclopropane fatty acid synthase 2 (SEQ ID NO.: 11), were synthesized using S. cerevisiae codon usage by GenScript USA Inc. 860 Centennial Ave., Piscataway, N.J. 08854, USA, flanked by SpeI and Not I restriction sites and cloned into the SpeI and Not I digested vector, pFBA1-413N (SEQ ID NO.: 13), resulting in plasmids pZ20 and pZ22, respectively. The heterologous desaturase ORFs are expressed under the control of S. cerevisiae fructose-biphosphate aldolase (EC 4.1.2.13; GenBank No.: X15003; YKL060C; FBA1) promoter (601 bp upstream of the FBA1 ORF), a ‘ctagtgccacc’ sequence containing the Kozak consensus sequence placed between the FBA1 promoter and the heterologous ORF, and the ADH1 terminator.

Transformation of an Isobutanologen with and Expression of Heterologous Cyclopropane Fatty Acid Synthases using a Yeast Expression Vector

Isobutanologen strain PNY2145 was co-transformed by the lithium acetate method (Methods in Yeast Genetics, 2005, page 113) with 0.5 μg each of pLH804::L2V4 (SEQ ID NO.: 76) and empty vector, pZ20 or pZ22. pLH804::L2V4 (SEQ ID NO.: 76) contains the K9JB4P variant of Anaerostipes caccae ILVC under the control of S. cerevisiae ILV5 promoter, and the L2V4 variant of Streptococcus mutans ILVD, under the control of S. cerevisiae TEF promoter. Transformants were selected on minimal medium plates containing 2% ethanol as carbon source. Two empty vector transformants (a, b) and four transformants (a-d) each of pZ20 and pZ22 were grown aerobically in PM in 24-well block at 30° C. An aliquot was used to start 5 mL PM cultures in 15 mL screw cap tunes and grown on a rotary drum for 4 days at 30° C. overnight in PM. Remaining aerobic cultures and all anaerobic cultures were harvested and the pellets analyzed for fatty acid composition.

Fatty acid profile of the average each of the four independent transformants and of the two vector only controls were analyzed by GC method to ascertain the proper expression of the cyclopropane fatty acid synthases. Cells were harvested, lipid extracted and GC analyzed as described in the General Method section. The result of the GC analysis are shown in Tables 18. In the cyclopropane fatty acid synthase transformants 1-3% of the cyclopropane fatty acids were synthesized aerobically and 2-5% anaerobically.

TABLE 18
Lipid profile of PNY2145 transformed with vector empty vector, L. plantarum
cyclopropane fatty acid synthase 1 and cyclopropane fatty acid synthase 2

| | | | | | | C19:0 |
|---|---|---|---|---|---|---|
| | | | | | | cyclopropane |
| Strain | C16:0 | C16:1 | C18:0 | C18:1 | C18:2 | fatty acid |
| Overnight in PM | | | | | | |
| tube, aerobic | | | | | | |
| empty vector | 11 | 40 | 6 | 40 | 0 | 0 |
| pZ20 (cfa1) | 11 | 41 | 6 | 39 | 0 | 1 |
| pZ22 (cfa2) | 12 | 40 | 7 | 36 | 0 | 3 |
| 4 days in PM tube, | | | | | | |
| anaerobic | | | | | | |
| empty vector | 9 | 49 | 5 | 31 | 0 | 0 |
| pZ20 (cfa1) | 8 | 47 | 6 | 31 | 0 | 2 |
| pZ22 (cfa2) | 12 | 44 | 6 | 27 | 1 | 5 |

### Example 6

### Co-Expression of FBA1:Yld9d and FBA1:Mad9d with M. alpina Fatty Acid Elongase

Strains C53 and C55 from Example 2 (Table 13) were transformed with copies of M. alpina fatty acid elongase (FBA1:Maelo) that were integrated into the genome. For this, DNA cassettes (SEQ ID NO: 82) comprised of delta sequences flanking the FBA1 promoter, M. alpina fatty acid elongase (SEQ ID NO: 18), the FBA1 terminator, and URA3 that is flanked by loxp66/loxp72 sequences were integrated into the genome. Cells were harvested, lipid extracted and GC analyzed as described in the General Method section. Results in Table 19 show that very high 18:1 levels (72% and higher) were achieved. Table 20 compares their conversion efficiencies.

TABLE 19
Total lipid profile of OLE1D::Yld9d (C32) transformed by FBA1:Yld9d,
FBA1:Yld9d + FBA1:Maelo, FBA1:Mad9d, or FBA1:Mad9d + FBA1:Maelo

| | FAC % Total | | | | | | Ratios | | |
|---|---|---|---|---|---|---|---|---|---|
| Strain | C16:0 | C16:1 | C16:2 | C18:0 | C18:1 | C18:2 | C18:1/C16:1 | C18/C16 | unsaturated/saturated |
| OLE1Δ::Yld9d | 11 | 60 | 0 | 2 | 27 | 0 | 0.5 | 0.4 | 7.0 |
| (C32) | | | | | | | | | |
| C32 + Yld9d | 11 | 54 | 1 | 1 | 33 | 0 | 0.6 | 0.5 | 7.3 |
| (C53) | | | | | | | | | |
| C53 + Maelo | 3 | 19 | 1 | 4 | 72 | 0 | 3.7 | 3.1 | 13.0 |
| C32 + Mad9d | 8 | 50 | 0 | 2 | 40 | 0 | 0.8 | 0.7 | 9.0 |
| (C55) | | | | | | | | | |
| C55 + Maelo | 2 | 17 | 0 | 3 | 78 | 0 | 4.5 | 4.3 | 18.7 |

TABLE 20
Conversion efficiency of OLE1Δ::Yld9d (C32) transformed by
FBA1:Yld9d, FBA1:Yld9d + FBA1:Maelo, FBA1:Mad9d, or
FBA1:Mad9d + FBA1:Maelo

| | d9d | d9d | d9d | |
|---|---|---|---|---|
| | c.e. on | c.e. on | c.e. on | |
| Strain | C16 | C18 | total | elo c.e. |
| OLE1Δ::Yld9d (C32) | 85 | 94 | 87 | 29 |
| C32 + Yld9d (C53) | 83 | 97 | 88 | 35 |
| C53 + Maelo | 86 | 95 | 93 | 76 |
| C32 + Mad9d (C55) | 86 | 96 | 90 | 42 |
| C55 + Maelo | 91 | 96 | 95 | 81 |

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1-63. (canceled)

64. A yeast microorganism comprising an engineered butanol biosynthetic pathway and an altered lipid profile, wherein the yeast microorganism comprises a different composition of fatty acids as compared to a wild-type yeast microorganism grown under standard fermentation conditions.

65. The yeast microorganism of claim 1, wherein the yeast microorganism is engineered to express one or more enzymes selected from the group consisting of fatty acid desaturase, fatty acid elongase, cyclopropane fatty acid synthase, or combinations thereof.

66. The yeast microorganism of claim 1, wherein the altered lipid profile comprises one or more of the following: (1) an increase in the concentration of C18:1, C18:2, and C18:3 fatty acids, (2) an increase in the ratio of unsaturated to saturated fatty acids, (3) an increase in the concentration of cyclopropane fatty acid, and (4) an increase in the C18 to C16 fatty acid concentration ratio, as compared to a microorganism that lacks an altered lipid profile.

67. The yeast microorganism of claim 65, wherein the fatty acid desaturase is selected from:
a) a polypeptide that has at least 90% identity to any one or more of SEQ ID NOs: 1, 2, or 9;
b) a polypeptide encoded by a nucleic acid sequence that has at least 90% identity to any one or more of SEQ ID NOs: 3, 4, or 10;
c) a fatty acid desaturase having an EC number 1.14.19.1 or 1.14.19.6; and
d) a fatty acid desaturase isolated from Yarrowia lipolytica, Fusarium moniliforme, or Mortierella alpine.

68. The yeast microorganism of claim 65, wherein the fatty acid elongase is selected from:
a) a polypeptide that has at least 90% identity to any one or more of SEQ ID NOs: 11, 15, or 16;
b) a polypeptide encoded by a nucleic acid sequence that has at least 90% identity to any one or more of SEQ ID NOs: 12, 17, or 18; and
c) a fatty acid elongase isolated from Euglena gracilis, Yarrowia lipolytica, or Mortierella alpine.

69. The yeast microorganism of claim 65, wherein the cyclopropane fatty acid synthase is selected from:
a) a polypeptide that has at least 90% identity to any one or more of SEQ ID NOs: 5 or 6;
b) a polypeptide encoded by a nucleic acid sequence that has at least 90% identity to any one or more of SEQ ID NOs: 7 or 8;
c) a cyclopropane fatty acid synthase having an EC number 2.1.1.79; and
d) a cyclopropane fatty acid synthase isolated from Lactobacillus plantarum.

70. The yeast microorganism of claim 64, wherein the yeast microorganism further comprises at least one modification selected from the group consisting of a modification in one or more polynucleotides encoding a polypeptide having pyruvate decarboxylase activity; a modification in one or more polynucleotides encoding a polypeptide having glycerol-3-phosphate dehydrogenase activity; a modification in one or more polynucleotides encoding a polypeptide having acetolactate reductase activity; a modification in one or more polynucleotides encoding a polypeptide having aldehyde dehydrogenase activity; and a genetic modification in FRA2.

71. The yeast microorganism of claim 64, wherein the engineered butanol biosynthetic pathway is an engineered isobutanol biosynthetic pathway.

72. The yeast microorganism of claim 71, wherein the engineered isobutanol biosynthetic pathway comprises the following substrate to product conversions:
a) pyruvate to acetolactate;
b) acetolactate to 2,3-dihydroxyisovalerate;
c) 2,3-dihydroxyisovalerate to α-ketoisovalerate;
d) α-ketoisovalerate to isobutyraldehyde; and
e) isobutyraldehyde to isobutanol;
and wherein
i) the substrate to product conversion of step (a) is performed by a recombinantly expressed acetolactate synthase;
ii) the substrate to product conversion of step (b) is performed by a recombinantly expressed acetohydroxy acid isomeroreductase;
iii) the substrate to product conversion of step (c) is performed by a recombinantly expressed acetohydroxy acid dehydratase;
iv) the substrate to product conversion of step (d) is performed by a recombinantly expressed branched-chain keto acid decarboxylase; and
v) the substrate to product conversion of step (e) is performed by an alcohol dehydrogenase;
whereby isobutanol is produced from pyruvate via the substrate to product conversions of steps (a)-(e).

73. The yeast microorganism of claim 72, wherein the acetolactate synthase is selected from
a) an acetolactate synthase having an EC number 2.2.1.6;
b) a polypeptide that has at least 90% identity to any one or more of SEQ ID NOs: 13, 14, or 19;
c) a polypeptide encoded by a nucleic acid sequence that has at least 90% identity to any one or more of SEQ ID NOs: 20, 21, or 22; and
d) an acetolactate synthase isolated from Bacillus subtilis, Klebsiella pneumonia, or Lactococcus lactis.

74. The yeast microorganism of claim 72, wherein the acetohydroxy acid isomeroreductase is selected from
a) an acetohydroxy acid isomeroreductase having an EC number 1.1.1.86;
b) a polypeptide that has at least 90% identity to any one or more of SEQ ID NOs: 65, 66, or 67; and
c) an acetohydroxy acid isomeroreductase isolated from Anaerostipes caccae, Lactococcus lactis, Vibrio cholera, Pseudomonas aeruginosa, or Pseudomonas fluorescens.

75. The yeast microorganism of claim 72, wherein the acetohydroxy acid dehydratase is selected from
a) an acetohydroxy acid dehydratase having an EC number 4.2.1.9;
b) a polypeptide that has at least 90% identity to any one or more of SEQ ID NOs: 30, 33, or 68; and
c) an acetohydroxy acid dehydratase isolated from Escherichia coli, Bacillus subtilis, or Streptococcus mutans.

76. The yeast microorganism of claim 72, wherein the branched-chain keto acid decarboxylase is selected from
a) a branched-chain keto acid decarboxylase having an EC number 4.1.1.72;
b) a polypeptide that has at least 90% identity to any one or more of SEQ ID NOs: 38, 69, or 70; and
c) a branched-chain keto acid decarboxylase isolated from Lactococcus lactis, M. caseolyticus, or L. grayi.

77. The yeast microorganism of claim 64, wherein the yeast microorganism is a member of a genus selected from Saccharomyces, Schizosaccharomyces, Hansenula, Candida, Kluyveromyces, Yarrowia, Issatchenkia, or Pichia.

78. A method of producing butanol from an engineered butanol biosynthetic pathway comprising:
a) providing the yeast microorganism of claim 64; and
b) growing the yeast microorganism under conditions whereby butanol is produced from pyruvate.

79. The method of claim 78, wherein the engineered butanol biosynthetic pathway is an isobutanol biosynthetic pathway.

80. The method of claim 79 further comprising c) recovering the isobutanol.

81. The method of claim 80, wherein the recovering is by distillation, liquid-liquid extraction, adsorption, decantation, pervaporation, or combinations thereof.

82. A bio-based fuel comprising butanol produced by the method of claim 78.
